SEQUENCE LISTING (14 sequences)

SEQ ID NO: 1 - DNA, 66 bp, Artificial Sequence
  Coding sequence for VK-1 signal peptide sequence
atggacatgagggtgcccgctcagctcctggggctcctgctgctgtggctgagaggtgcgcgctgt

SEQ ID NO: 2 - protein, 22 aa, Artificial Sequence
  VK-1 signal peptide sequence
MDMRVPAQLLGLLLLWLRGARC

SEQ ID NO: 3 - DNA, 19 bp, Artificial Sequence
  Primer sequence
gacagccccgattcttcca

SEQ ID NO: 4 - DNA, 25 bp, Artificial Sequence
  Primer sequence
tcttccttttttgtggcactattct

SEQ ID NO: 5 - DNA, 27 bp, Artificial Sequence
  Probe sequence
cccaaaggcaaacaacctactgctgca

SEQ ID NO: 6 - DNA, 21 bp, Artificial Sequence
  Primer sequence
acccacactgtgcccatctac

SEQ ID NO: 7 - DNA, 25 bp, Artificial Sequence
  Primer sequence
gctcagtgaggatcttcatgaggta

SEQ ID NO: 8 - DNA, 20 bp, Artificial Sequence
  Probe sequence
ctggctggccgggacctgac

SEQ ID NO: 9 - DNA, 22 bp, Artificial Sequence
  Primer sequence
tgctcatcaacctgactttgga

SEQ ID NO: 10 - DNA, 22 bp, Artificial Sequence
  Primer sequence
cactgctcccattcatcagttc

SEQ ID NO: 11 - DNA, 28 bp, Artificial Sequence
  Probe sequence
ttctgggatgcaactgagattccaacct

SEQ ID NO: 12 - DNA, 23 bp, Artificial Sequence
  Primer sequence
cccatgcattcaaactgaggtaa

SEQ ID NO: 13 - DNA, 18 bp, Artificial Sequence
  Primer sequence
tggcctgcccggttatta

SEQ ID NO: 14 - DNA, 19 bp, Artificial Sequence
  Probe sequence
tccagctgagcgccggtcg

(19) United States
(12) Patent Application Publication
(10) Pub. No.: US 2015/0201588 A1
(43) Pub. Date: Jul. 23, 2015

(54) Autologous Mammalian Models Derived from Induced Pluripotent Stem Cells and Related Methods

(71) Applicant: AMGEN INC., Thousand Oaks, CA (US)

(72) Inventors: Carl Alexander Kamb, Hillsborough, CA (US); Helen Y. Kim, Westlake Village, CA (US); Sungeun Kim, San Mateo, CA (US); William Christian Fanslow, III, Normandy Park, CA (US)

(73) Assignee: AMGEN INC., Thousand Oaks, CA (US)

(21) Appl. No.: 14/466,844

(22) PCT Filed: Feb. 22, 2013
(86) PCT No.: PCT/US2013/027479

§ 371 Date: Aug. 22, 2014

Related U.S. Application Data
(60) Provisional application No. 61/602,044, filed on Feb. 22, 2012.

(51) Int. Cl. A01K 67/027; C12Q 1/68; C12N 5/071

(52) U.S. Cl. CPC A01K 67/0271; C12N 5/0679; C12Q 1/6876; C12N 2506/45; C12N 2501/16; C12N 2501/415; C12N 2501/119; C12N 2502/13; A01K 2227/106; A01K 2267/03

(57) ABSTRACT

Disclosed is an autologous non-human mammalian model system derived from induced pluripotent stem (iPS) cells. Also disclosed are methods of differentiating non-human primate iPS cells, which can result in populations of cells enriched for SOX2+ or PDX1+ foregut-like cells, for CDX2+ hindgut-like cells, for CD34+ hematopoietic progenitor-like cells, or epithelial-like cells. Also disclosed is a non-human primate containing an autologous cell type of interest, which is differentiated in vitro from an induced pluripotent stem cell reprogrammed from a primary somatic cell. Methods of monitoring exogenously introduced cells within a non-human mammal are also disclosed.

This application claims the benefit of U.S. Provisional Application No. 61/602,044, filed Feb. 22, 2012, which is hereby incorporated by reference in its entirety.

The instant application contains an ASCII “txt” compliant sequence listing submitted via EFS-WEB on Feb. 22, 2013, which serves as both the computer readable form (CRF) and the paper copy required by 37 C.F.R. Section 1.821(c) and 1.821(e), and is hereby incorporated by reference in its entirety. The name of the “txt” file created on Feb. 20, 2013, is: A-1652-WO-PCTSeqList022013_ST25.txt, and is 4 kb in size.

Throughout this application various publications are referenced within parentheses or brackets. The disclosures of these publications in their entireties are hereby incorporated by reference in this application in order to more fully describe the state of the art to which this invention pertains.

### BACKGROUND OF THE INVENTION

1. Field of the Invention

The present invention is directed to the field of animal models of disease.

2. Discussion of the Related Art

Results of experiments in model organisms are used to help predict safety and efficacy of therapeutic molecules in humans. Rodent disease models are often convenient due to their relative ease of housing and care, and their tractability for molecular genetic engineering and breeding.

However, comparatively little effort has been applied to development of non-human primate (NHP) models in important diseases such as inflammation and cancer. Xenogeneic and allogeneic responses to transplanted cells and tissue obfuscate disease-relevant biology—especially efficacy that may be mediated in part by immune effector mechanisms (Gomez-Roman, V. R. et al., A simplified method for the rapid fluorometric assessment of antibody-dependent cell-mediated cytotoxicity, J Immunol Methods 308, 53-67 (2006); Gomez-Roman, V. R. et al., Vaccine-elicited antibodies mediate antibody-dependent cellular cytotoxicity correlated with significantly reduced acute viremia in rhesus macaques challenged with SIVmac251, J Immunol 174, 2185-2189 (2005); Vowels, B. R. et al., Natural killer cell activity of rhesus macaques against retrovirus-pulsed CD4+ target cells, AIDS Res Hum Retroviruses 6, 905-918 (1990)).

Autologous non-human mammalian model systems are needed for drug development. Also, given the similarity of immune effector components in non-human primates compared to humans, an autologous non-human primate model is a particular desideratum. These and other benefits the present invention provides.

### SUMMARY OF THE INVENTION

The present invention involves an autologous non-human mammalian model system. In particular embodiments the non-human mammal is one commonly used in biomedical research, e.g., a rodent, a rabbit, a dog, a cat, a pig, a sheep, or a non-human primate (e.g., cynomolgus macaque) model system (FIG. 1).

Cynomolgus monkeys (also known as “cynos”) are macaques (Macaca fascicularis synonym M. cynomolgus) of southeastern Asia, Borneo, and the Philippines that are often used in medical research. Cynos and their close relatives differ from humans by about 7% at the DNA level. More importantly, the immune systems of these non-human primates (NHPs) are similar to human immune systems. Thus, the cyno is a particularly useful subject for the development of predictive disease models.

The autologous non-human mammalian (or primate) model system involves introducing (e.g., by injection or implantation or infusion) into a non-human mammal (or primate) an autologous cell type of interest, which is differentiated from an induced pluripotent stem cell reprogrammed (fully or partially reprogrammed) from a primary somatic cell obtained from the non-human mammal (or primate). The “cell type of interest” can encompass an effector cell(s) or a target cell(s) or a plurality of cells comprised in a graft (e.g., a malignant graft or tumor, or a non-malignant graft or tissue). In some embodiments, the graft is grown in the autologous mammal (such as the autologous non-human primate). In other embodiments, the graft is grown in the autologous mammal, removed and expanded in vitro, then retransplanted into the autologous mammal. In alternative embodiments, the graft is grown first in another mammal before being transplanted back into the autologous mammal (such as the autologous non-human primate).

Subsequently, in the autologous non-human mammalian (or primate) model system, a therapeutic candidate is administered to the non-human mammal (or primate); and then a physiological effect, if any, of the therapeutic candidate is determined in the non-human mammal (or primate), by the use of a suitable assay or other assessment tool depending on the physiological process, disease indication or condition of interest.

For example, we generated autologous cynomolgus (cyno) macaque (Macaca fascicularis) target cells, which allow immune effector therapeutics (e.g., bi-specific T-cell engagers [BiTE®], bi-specific killer cell engager or a [BiKE], or ADCC)-mediated efficacy and toxicology studies in the autologous non-human primate settings. We first isolated fibroblasts from individual cyno monkeys and reprogrammed them to induced pluripotent stem (iPS) cells. These autologous cyno iPS cells were further differentiated into multiple target cells to generate autologous target cell types of interest for functional ADCC assays in vitro. Specific genes including ADCC candidate genes and/or reporter genes for tracking in vivo were transduced into the autologous target cells, as described in more detail herein. These cells carrying the gene of interest can be transplanted back into the original donor cyno monkeys to test ADCC-mediated efficacy and toxicology of therapeutic antibodies in this autologous in vivo setting. Specifically, cyno monkeys bearing the autologous cells or grafts can be treated with a therapeutic candidate molecule targeting a gene product of interest expressed by the cells or grafts, and the target cell clearance can then be monitored by various methods known in the art or described herein. This generation of autologous preclinical primate models using the iPS cell technology can be a reliable, efficient strategy for development of therapeutics, and has broad applicability for various diseases, including cancer and autoimmunity.

Some embodiments of the invention include the generation of tumor-like target cells that express a tumor-selective antigen for testing antibodies designed to deplete or kill tumor cells with these properties; or generation of target cells that mimic normal, but rare and difficult to track cells, and cells that are thought to contribute to inflammatory diseases which may be targeted by specific depleting antibodies. In each of these cases, the autologous target cells are introduced into the NHP recipient and monitored using techniques known in the art, under various conditions such as administration of a therapeutic candidate or tool compound.

We have developed methods that allow generation of autologous cells and grafts of predetermined and controlled types, which can be reintroduced into the original donor animals for further experimentation; specifically tests of safety and efficacy of therapeutic candidate drugs or tool compounds. These cells can not only be generated to mimic normal somatic and malignant cells of defined types, but also can be engineered to express specific genes, some of which may be chosen to facilitate tracking of the cells in vivo, quantification of cell number, or targeting of specific therapeutic or tool compounds.

For example, one embodiment of the present invention includes a method of differentiating non-human primate induced pluripotent stem (iPS) cells, in vitro, which involves incubating or culturing the iPS cells in a cell culture medium comprising a concentration of activin A (10-150 ng/ml, preferably 50-120 ng/ml, more preferably 90-110 ng/ml); the concentration of serum in the medium is increased from serum-free to about 0.2% (±0.1% (v/v)) in the first day (i.e. during the first 24±6 hours) and to a final concentration of about 2% (±1% (v/v)) from the second day onward (i.e., after the first day), effective to induce differentiation of definitive endoderm (DE) cells. The DE cells are characteristically FOXA2⁺, SOX17⁺ (see, FIG. 9A). Continuing to culture these cells in cell culture medium comprising the same concentration of activin A and the same final concentration of serum (about 2%±1% (v/v)), for a period of at least twelve days, results in a population of cells enriched to greater than 90%, 91%, 92%, 93%, 94%, 95%, 96%, 97%, 98%, or 99% for SOX2⁺ or PDX1⁺ foregut-like cells.

Another embodiment of a method of differentiating non-human primate induced pluripotent stem (iPS) cells, in vitro, involves incubating or culturing the iPS cells for about three days (i.e., 72 hours±6 hours) in a cell culture medium comprising a concentration of activin A (10-150 ng/ml, preferably 50-120 ng/ml, more preferably 90-110 ng/ml), while increasing the concentration of serum in the medium from serum-free to about 0.2% (±0.1% (v/v)) in the first day (i.e. during the first 24±6 hours) and to a final concentration of about 2% (±1% (v/v)) from the second day onward (i.e., after the first day), effective to induce differentiation of definitive endoderm (DE) cells. This culture regimen of about three days, is then followed by incubating or culturing the cells in a cell culture medium comprising a concentration of Wnt3a (100-1000 ng/ml, preferably 400-600 ng/ml, more preferably 450-550 ng/ml), a concentration of FGF4 (100-1000 ng/ml, preferably 400-600 ng/ml, more preferably 450-550 ng/ml; which can be same or different from the Wnt3a concentration), and the same final concentration of serum (about 2%±1% (v/v)), without added activin A, for a period of at least nine days. This results in a population of cells enriched to greater than 90%, 91%, 92%, 93%, 94%, 95%, 96%, 97%, 98%, or 99% for CDX2⁺ hindgut-like cells.

Another embodiment of a method of differentiating non-human primate induced pluripotent stem (iPS) cells, in vitro, involves co-culturing the iPS cells with stromal cells for at least about thirty days; a population of cells results that is enriched to greater than 10%, 11%, 12%, 13%, 14%, 15%, or 16% for CD34⁺ hematopoietic progenitor-like cells.

In still another embodiment of a method of differentiating non-human primate induced pluripotent stem (iPS) cells, in vitro, the method involves incubating or culturing the iPS cells in a cell culture medium comprising a serum concentration of about 10% (±2% (v/v)), which results in a population of epithelial-like cells.

Another embodiment of the invention is a method of monitoring exogenously introduced cells within a non-human mammal, which involves introducing into a non-human mammal, such as but not limited to a non human primate (e.g., Macaca fascicularis), a recombinant cell that expresses a reporter gene (e.g., a Gaussia princeps luciferase (Gluc) gene, or another exogenous or endogenous gene of interest the expression of which can be detected by measuring specific mRNA using real time PCR (qPCR) or PCR), or nucleic acid sequencing, or flow cytometry, or protein-based detection assay, or immunoassay, or another suitable detection assay known in the art; and detecting the reporter gene activity in a tissue sample (e.g., a blood sample [including whole blood, serum or plasma], or sample of a non-malignant or malignant graft or a malignant tumor sample) obtained from the non-human mammal; the level of reporter gene activity is correlated to the number of recombinant cells present in the non-human mammal.

In another embodiment of a method of monitoring exogenously introduced cells within a non-human mammal, the method involves introducing into a non-human mammal a recombinant cell that comprises an exogenous gene of interest; and detecting genomic DNA that is specific to the exogenous gene of interest in a tissue sample (e.g., a blood sample or graft sample) obtained from the non-human mammal, wherein the level of genomic DNA that is specific to the exogenous gene of interest is correlated to the number of recombinant cells present in the non-human mammal.

Further embodiments of the invention include a non-human primate containing a target cell type of interest (e.g., epithelial-like, hematopoietic-like cell, neuron-like cell, cardiomyocyte, foregut-like cell, midgut-like cell, hindgut-like cell, or mesenchymal-like cell) or effector cell type of interest (e.g., NK cell, T cells, macrophage, monocyte, or neutrophil) differentiated in vitro from an induced pluripotent stem cell reprogrammed from a primary somatic cell previously obtained from the non-human primate. For example, in some embodiments, the non-human primate comprises a SOX2⁺ or PDX1⁺ foregut-like cell or a CDX2⁺ hindgut-like cell, which is differentiated in vitro by the inventive method of differentiating non-human primate induced pluripotent stem (iPS) cells.

Among various methods to generate the autologous cyno target cell type of interest, the iPS (induced pluripotent stem) cell-derived approach can provide a very useful tool to generate target cells that are typically difficult to obtain from live animals, such as endoderm derivatives, including stomach, lung, pancreas, liver, intestine, and colon, and neurons.

Herein, we demonstrate that NHP somatic cells can be reprogrammed to autologous iPS cells, which can be further differentiated into various autologous target cell types and autologous effector cell types of interest, which can then be reintroduced to the NHP, and methods of monitoring exogenously introduced cells, including such autologous cells, which are all applicable to model systems directed to a broad range of disease indications to which new therapeutics are sought.

### BRIEF DESCRIPTION OF THE DRAWINGS

FIG. 1 is a schematic overview depicting the generation of an autologous non-human primate preclinical model using iPS cell-derived autologous cells to test a therapeutic candidate compound, e.g., antibodies. This autologous model development starts with generating iPS cells from non-human primate (e.g., monkey) primary somatic cells, such as skin fibroblasts and PBMC which can be readily obtained from live animals. Continuing clockwise, these differentiated adult somatic cells can be reprogrammed into a pluripotent state by ectopic expression of four transcription factors, Oct4, Sox2, Klf4, and c-Myc. These iPS cells can differentiate into various types of target cells. This approach can provide various autologous target cells of interest in sufficient amounts. Next, ADCC candidate genes can be introduced into the specific target cells. These autologous target cells carrying a gene of interest (“gene X”) can be transplanted back into the original donor animal to examine efficacies and toxicology of therapeutic antibodies (against gene X) for their potential ADCC activities in this autologous setting.

FIG. 2A-B shows transduction efficiency of the retrovirus carrying four transcription factors in cyno skin fibroblasts. FIG. 2A shows immunostaining analysis for expression of the four indicated transcription factors, OCT4, KLF4, c-MYC, and SOX2 proteins. Transduction efficiency of the retrovirus (pMX-based vector) carrying these four factors in cyno skin fibroblasts was examined. Dapi (in top row) was used to stain the cellular nuclei. FIG. 2B shows quantification of the expression of four transcription factors based on the immunofluorescence images (n=3). Retroviruses from two different backbone plasmids (pMX and pBMN) were tested.

FIG. 3 illustrates morphological changes of cyno skin fibroblasts isolated and expanded from dorsal skins of cyno monkeys (upper left panel) upon reprogramming into cyno iPS cells. Upon transduction with retroviral vectors carrying coding sequences for four transcription factors (OCT4, SOX2, KLF4, and c-MYC), the cyno fibroblasts underwent the drastic changes in morphology, and began to divide into large spherical clusters of ES-like colonies. They formed three different types of colonies: type I (upper right panel), type II (lower left panel), and type III (lower right panel). Analysis of ES cell-like properties of these different types of colonies demonstrated that the Type III iPS lines were fully reprogrammed cyno iPS cells where as Type I and Type II iPS lines were partially reprogrammed cyno iPS cells. Micrograph scale bar=1000 μm.

FIG. 4 shows ES cell-like properties of cyno iPS cell lines compared to the parental cyno skin fibroblasts. FIG. 4 (upper row) illustrates that cyno iPS colonies (Cyno iPS11 and Cyno iPS26) showed homogeneous ES cell-like morphology that resembles that of human iPS cells shown here as a positive control. FIG. 4 (lower row) illustrates that cyno iPS cell lines in later passages showed homogeneous populations with alkaline phosphatase (AP)+ colonies, as shown in human iPS cells, whereas the parental cyno skin fibroblasts did not express AP. Micrograph scale bar=1000 μm.

FIG. 5A-C illustrates validation of pluripotency marker expression in a reprogrammed cyno iPS cell line. Immunofluorescence staining of (left to right in each row) TRA-1-60, SSEA-4, and NANOG pluripotency markers, which were highly expressed in human iPS cells (FIG. 5A) and fully reprogrammed iPS cell line (cyno iPS 11; FIG. 5B). Differentiated cyno colonies failed to express any of these pluripotency proteins (FIG. 5C). The right-most panel in FIG. 5A-C shows DAPI-stained cells; DAPI was used to stain the cellular nuclei.

FIG. 6A-D shows differential potential of reprogrammed cyno iPS cells into Multiple Cell Types. Embryoid body (EB)-mediated differentiation of cyno iPS cells demonstrated differential potential of reprogrammed cyno iPS cells into multiple cell types including all three germ layer lineages (ectoderm, mesoderm, and endoderm) (FIG. 6A-C). EBs derived from cyno iPS cells (cyno iPS lines 11 and 26; two middle micrographs in each of FIGS. 6A-C) were transferred into gelatin-coated plates to grow in serum-containing media. These differentiated cyno iPS cells were immuno-stained for tissue/cell type-specific markers. The parental cyno skin fibroblasts (rightmost micrograph in FIG. 6A-C) did not display a differential potential to any of lineages (FIG. 6A-C). A human iPS cell line was used as a positive control for the differentiation and immunofluorescence staining (leftmost micrograph in FIG. 6A-C). FIG. 6A illustrates that neuronal axons (ectoderm) were differentiated from cyno iPS cells, evidenced by immunostaining of βIII-tubulin. FIG. 6B illustrates that mesodermal cells were differentiated from cyno iPS cells, as indicated by α-Smooth Muscle Actin (SMA) immunostaining FIG. 6C shows that endodermal intestinal tissues (“bright field” micrograph) with canal-like structures were differentiated from cyno iPS cells which were demonstrated by immunostaining of CDX2 (specific for hindgut lineages). FIG. 6D shows cardiomyocytes were differentiated from cyno iPS cells, as evidenced by beating heart cells (motion not shown).

FIG. 7A-C shows results from the characterization of three different morphological types of cyno iPS colonies (Type I, II, and III). Immunofluorescence analysis of pluripotency markers showed that type I cyno iPS colonies (clones) were TRA-1-60⁺ SSEA-4⁻ Nanog⁺ Oct4⁺, and type II cyno iPS clones were TRA-1-60⁻ SSEA-4⁻ Nanog⁺ Oct4⁻, and type III cyno iPS clones were TRA-1-60⁺ SSEA-4⁺ Nanog⁺ Oct4⁺ (FIG. 7A). The cyno fibroblasts which were parental lines for reprogramming were used as negative control cells (FIG. 7A; “Cyano Fibroblast 1503” is shown as a representative). The Nanog mRNA expression was analyzed by real Time PCR (qPCR) acquiring the relative quantification [RQ=2̂−(ΔΔCt)] relative to cyno iPS 11 line (FIG. 7B). The Cyno iPS 11 line is a fully validated iPS cell lines (type III) which can be used as a calibrator sample. Each sample was also normalized against β-actin as an internal control to generate ΔCt. The differentiation potential of these different types of cyno iPS clones was examined by generating EBs (FIG. 7C). EB-derived differentiation assays showed that the type III cyno iPS clones possess the differential potential into all three germ layer lineages, whereas type I and type II cyno iPS clones were able to differentiate into ectoderm and mesoderm, but not endoderm (FIG. 7C).

FIG. 8 illustrates generation and characterization of cyno epithelial-like cells derived from autologous cyno iPS cells. One of the strategies to generate autologous cyno target cells was the differentiation of cyno iPS cells into (heterogeneous) epithelial-like cells (cyno iPS-EPI cells). A single cyno iPS cell line or multiple (pooled more than two) cyno iPS cell-like lines was used to differentiate into epithelial-like cells (cyno iPS-EPI-1 or cyno iPS-EPI-3, respectively). 1504 and 1509 represent cyno monkey ID numbers. We characterized the expression of epithelial cell-specific markers, pan-cytokeratin (“pan-CK”) in the cyno iPS-EPI cells. Immunofluorescence analysis revealed that cyno iPS-EPI contained cells with high expression of CKs, similar to SK-BR-3, a luminal breast cancer cell line that was used as a positive control for high expression of pan-CK. Cyno skin fibroblasts were used as a negative control for pan-CK staining DAPI was used for nuclei stain. Scale bar=100 μm

FIG. 9A-B shows generation of mouse target cells by differentiation of mouse iPS Cells into definitive endoderm (“DE”). FIG. 9A shows immunofluorescence staining for the definitive endoderm markers FOXA2 and SOX17 (two leftmost columns, respectively), indicating that mouse iPS cells (miPS 16 and 36 lines) grown in a high concentration of activin A can differentiate into definitive endoderm. Controls were immunostained with DAPI and MERGE (two rightmost columns, respectively). Quantitative analyses of immunofluorescence images demonstrated that DE cells co-expressing the definitive endoderm markers FOXA2 and SOX17 were highly enriched by direct endoderm differentiation method (FIG. 9B) (n=3). Mouse ES cells (mES) used as a positive control, and mouse fibroblasts (mfibroblast) was used as a negative control.

FIG. 10 illustrates schematically various differentiation methods to enrich specific gut-like cells by differentiation of cyno iPS cells. Several different methods (rows A-F; see, Example 1 herein) were tested to differentiate cyno iPS cells and enrich for specific cyno gut lineage cells. Various conditions consist of different growth factors, compounds, induction timing and duration of treatment. Wnt3a and FGF4 were used as posteriorizing factors, Noggin as a physiological inhibitor of BMP signaling, and SB-431542 as a pharmacological inhibitor of activin A/nodal and TGF-β signaling.

FIG. 11 shows generation of cyno hindgut-like target cells derived from cyno iPS cells. Using immunofluorescence staining and imaging, the expression of gut-specific markers including CDX2 (middle column) as a hindgut marker and PDX1 as a foregut marker was characterized upon differentiation of cyno iPS cells under various growth factor conditions. Compared to methods D and F (see, FIG. 10), the method C (FIG. 10), in which cyno iPS cells were treated with activin A and a gradual increase in serum concentration for 3 days and then were treated with Wnt3a and FGF4, promoted the differentiation of cyno iPS cells into cyno DE and further hindgut-like cells. Thus method C resulted in high enrichment of hindgut-like cells (CDX2+ intestinal epithelial-like cells), and almost no foregut-like cells (˜0% of SOX2+ epithelial-like cells), indicating hindgut specification. Micrograph scale bar=100 μm.

FIG. 12 illustrates the generation of cyno foregut-like target cells derived from cyno iPS cells. Immunofluorescence staining and imaging revealed that the continuous treatment of cyno iPS cells with a high concentration (100 ng/ml) of activin A induced DE formation after 3 days (see, FIG. 10, method B), which led to high enrichment (˜93%) of cyno foregut-like cells (SOX2+ or PDX1+ cells) and almost no hindgut-like cells (˜0% of CDX2+ cells), indicating cyno foregut specification upon cyno iPS cell differentiation. Method A (FIG. 10) with no activin A failed to generate a high enrichment of gut-specific cells. The parental cyno skin fibroblasts failed to differentiate into any of gut-specific cells, evidenced by no expression of the gut-specific markers under any differentiation conditions (methods A-F in FIG. 10), confirming no differential potential of the fibroblast cells. Micrograph scale bar=100 μm.

FIG. 13 shows that the ability of cyno iPS cells to give rise to CD34⁺ hematopoietic progenitor-like cells (HPCs). Cyno iPS and human iPS cells were co-cultured with mouse bone marrow-derived stromal cells (M2-10B4). Flow cytometry analysis revealed that 11-16% cyno CD34⁺ hematopoietic progenitor-like cells and 0.6-3% of CD45+ leucocytes were differentiated from three cyno iPS lines at day 32 of co-culture (cyno iPS cell lines 11, 26, and 55, bottom row). Co-culture of human iPS cell line with M2-10B4 did not lead to efficient generation of CD34⁺ HPCs (˜3.4%) from human iPS cells. As expected, undifferentiated cyno iPS cells used as a negative control contained a very low frequency (˜0.3%) of CD34⁺ cells and (˜0.01%) CD45+ leucocytes. For negative controls, cyno iPS cells with an isotype control antibody and undifferentiated Cyno iPS 11 (top row) were also immunostained with a mixture of IgG-FITC (eBioscience), PE (eBioscience), PE-Cy5(eBioscience), APC (eBioscience), and APC-Cy7 (BD Biosciences). Human iPS cells were also compared (bottom row, left).

FIG. 14A-B shows detection of secreted Gluc activities from the cyno iPS-derived cells. To track the development of the iPS-derived target cells, secreted Gluc activities were detected in the iPS-derived epithelial-like cells (cyno iPS-EPI-1509-1 cell line from cyno monkey #1509). The cyno iPS-EPI cells that were transduced with Gluc-lentivirus for constitutive Gluc expression (“cyno iPS-EPI-1509-1_Gluc”) were examined along with the parental line (“cyno iPS-EPI-1509-1”) without Gluc expression. In FIG. 14A, conditioned media from different numbers of cyno iPS-EPI cells expressing Gluc were assayed with coelenterazine for Gluc activities after 24 h of culture (n=3). In FIG. 14B, conditioned media with cyno iPS-EPI cells (20,000 cells) expressing Gluc were assayed with coelenterazine for Gluc activities at different time points of culture (n=3).

FIG. 15A-C shows expression of exogenous and endogenous ADCC target genes (cell surface antigens) from various cyno iPS-derived target cells and cyno monkeys. In FIG. 15A, flow cytometry analyses were performed to examine target gene (Gluc) expression in the cyno target cells. Similar levels of expression of the ADCC target genes including exogenous CD20 and endogenous Her2 were detected from different cyno monkeys (“#1504” and “#1509”) and various cyno iPS-EPI cell lines (cyno iPS-EPI-1 and cyno iPS-EPI-3 per monkey). The parental lines without CD20 transduction were used as negative controls for CD20 staining. The unstained lines were used as negative lines for Her2 and CD20 staining. In FIGS. 15B and 15C, the quantitative analysis of the cell surface antigen expression (Her2 and CD20 target genes) was performed by QIFIKIT®-based flow cytometry. High cell surface expression of exogenous Her2 was detected in various cyno iPS-EPI-SP-Her2 lines from both cynos 1504 and 1509 (FIG. 15B). High cell surface expression of exogenous CD20 was detected in various cyno iPS-EPI-CD20 from both cynos 1504 and 1509 (FIG. 15C). Human breast cancer line, SKBR3 and human gastric cancer line, SNU620, were used for positive control lines for Her2 expression and a negative control for CD20 expression. Human Burkitt's lymphoma cell line, Daudi, was used for positive control for CD20 expression and a negative control for Her2 expression.

FIG. 16 shows cyno NK sensitivity (antibody independent cellular cytotoxicity, AICC) of various cyno target cell lines (iPS-EPI lines and their derivatives) in the absence of antibody. Cyno NK cells (CD159a⁺ cells enriched from cyno peripheral blood mononuclear cells (PBMC)) were used as effector cells. Despite the donor variability in NK effector cells, most of target cells showed a low level of NK-mediated specific lysis (AICC). N=1 indicates a unique cyno donor (n=2 to n=10 in total). CFSE (CFDA-SE, carbofluorescein diacetate succinimydil ester) (Invitrogen cell tracking kit, V 12883) labeled targets were incubated with NK cell enriched effector cells in a 5:1 effector:target ratio (E:T) for 18 hours.

FIG. 17A-B demonstrates the assessment of the ability of anti-Her2 (“aHer2”) huIgGI antibodies (wild type [“WT”] and afucosylated [“afuco”]) to induce cyno NK-mediated antibody-dependent cellular cytotoxicity (ADCC) against iPS-EPI target cells. As the cyno iPS-EPI-1509-3 line expresses a moderated level of endogenous Her2, only anti-Her2 Afuco was able to induce the potent NK cell-mediated ADCC against cyno iPS-EPI targets, whereas anti-Her2 WT and negative control huIgG1 failed to do so (FIG. 17A). However, when the cyno iPS-EPI-1509-3 line was further engineered to express an exogenous Her2 at the high cell surface expression level, both anti-Her2 WT and anti-Her2 Afuco were able to induce NK-mediated ADCC against the target cells (the cyno iPS-EPI-1509-3-Gluc/TetR/SP-Her2) (FIG. 17B).

FIG. 18 shows the evaluation of the ability of anti-Her2 (“aHer2”) huIgGI antibodies (wild type [“WT”] and afucosylated [“afuco”]) and anti-CD20 huIgGI antibodies to induce cyno NK-mediated ADCC against cyno iPS-EPI target cells. The cyno iPS-EPI-1509-1-Gluc/CD20 cell line was used as a target cell line expressing a high level of exogenous CD20 as well as a moderate level of endogenous Her2. Anti-Her2 Afuco was able to mediate potent cyno NK-mediated ADCC against the cyno iPS-EPI-1509-1-Gluc/CD20 cells due to the moderate level of Her2 endogenous expression in the target cells. In addition, an anti-CD20 Afuco led to increased cyno NK-mediated ADCC activities against the target cells-expressing exogenous CD20 cells at the lower levels of antibody concentration, compared to anti-CD20 WT.

FIG. 19A-B shows the evaluation of the effect of oncogenic transformation of multiple cyno iPS-EPI cell lines on the growth and survival ability in immunodeficient NSG (NOD scid gamma) mice. The cyno iPS-EPI cell lines were transduced with one or more oncogenes (e.g. HRas and/or SV40 large T antigen) and/or TERT (telomerase reverse transcriptase catalytic subunit), and/or anti-apoptotic genes (e.g. Bcl-xL). Either single or double transduction of iPS-EPI cells was carried out using cyno iPS-EPI-1504-1 cell line from cyno 1504 (FIG. 19B) and using iPS-EPI-1509-3 cell line from cyno 1509 (FIG. 19A) by retrovirus carrying HRas, Bcl-xL, and/or dogTert to generate diverse transformed cell lines (FIGS. 19A and 19B).

FIG. 20 demonstrates the immunohistochemical (IHC) staining for SV40 LT antigen to monitor and confirm the presence of exogenously introduced cyno iPS-EPI (Cyno iPS-EPI-1509-3.dTert+Bclxl) cells in grafts grown in NSG mice. IHC staining was performed on formalin-fixed paraffin embedded (FFPE) tissues. Using the SV40 LT IHC staining technique, SV40 LT-positive cell nuclei were stained dark brown with SV40LT antibody/Cardassian DAB chromagen (Biocare Medical #DBC859L10). SV40LT-negative nuclei were stained dark blue and all cytoplasm was stained light blue with the hematoxylin counterstain. The majority of the cyno iPS-EPI-1509-3.dTert+Bclxl graft cells were viable and demonstrated robust nuclear expression of SV40 LT antigen (as indicated by dark brown nuclear staining, but shown here as dark grey). Serial tissue sections of the same tissue region stained with hematoxylin and eosin (H&E) are presented. Both low (10×) and high (40×) magnifications are shown.

FIG. 21 shows the western blot analysis for the comparison between various cyno iPS-EPI cell lines and grafts derived from those cell lines grown in NSG mice for epithelial and mesenchymal cell marker expression. Cytokeratins and E-cadherin were used as epithelial cell markers. N-cadherin was used as a mesenchymal cell marker. Vimentin and SMA were used as both epithelial and mesenchymal cell markers. β-Actin was used as a loading control.

FIG. 22 shows graft formation of autologous cyno iPS-EPI-1509-3.HRas cells injected into cyno monkey 1509. The cyno iPS-EPI cell line was generated by reprogramming of skin fibroblasts obtained from cyno 1509 and then further engineered by transduction with retrovirus carrying a HRas oncogene to enhance proliferation and promote tumorigenicity, and ultimately to improve survival in cyno in vivo. The cyno iPS-EPI-1509-3.HRas cells were re-injected into the donor cyno 1509. The top (left column) and side (middle column) views of grafts and the length of graft in ultrasound (right column) measurements (length, width, and height) were shown as examples of graft measurement on day 18 and day 25 post cell injection.

FIG. 23A shows the ultrasound measurement of the cyno iPS-EPI-1509-3.HRas graft that was grown in NSG mice from cell injection and then was implanted into the autologous cyno 1509. Pre-implant measurement at day 1* was done by calipers. Cyno iPS-EPI-1509-3-HRas graft maintained the similar size to the original graft for 4 weeks post implantation. FIG. 23B shows the original cyno iPS-EPI-1509-3.HRas graft removed from NSG mice at day 1 (pre-implantation), and the top and side views of the graft implanted in the autologous cyno 1509 on day 11, day 21 and day 28 post-graft implantation. FIG. 23C shows the ultrasound imagest of the cyno iPS-EPI-1509-3.HRas graft that was grown from NSG mice and then was implanted into the autologous cyno monkey 1509. The cyno graft was measured by ultrasound on day 11, day 21 and day 28 post-graft implantation. The graft lengths shown in the panels of FIG. 23C are representative ultrasound measurements for the purpose of illustration.

FIG. 24A-B illustrates the detection of mRNA expression of iPS-EPI specific genes (exogenous SV40 LT mRNA [FIG. 24A] and exogenous Oct4 mRNA [FIG. 24B]) using RNA isolated from the cyno graft that was removed from cyno monkey 1509, to confirm the presence of cyno iPS-EPI-1509-3.HRas cells in the cyno grafts that were implanted into the cyno. The mRNA expressions of SV40 LT (FIG. 24A) and exogenous Oct4 (FIG. 24B) expression were analyzed by real Time PCR (qPCR) acquiring the relative quantification (RQ) relative to cyno 1509 fibroblasts (a negative control). The RNA isolated from the cyno iPS-EPI-1509-3.HRas graft that was grown in NSG mice (“NSG”) was used as a positive control.

FIG. 25A-B shows the evaluation of B6 mouse iPS cells-derived semi-autologous (syngeneic) models as a proof of concept. Using three different muiPS-EPI lines (muiPS-EPI-2A, muiPS-EPI-2B, and muiPS-EPI-2C), the abilities of the cell lines to grow and form grafts in syngeneic B6 mice were assessed. The muiPS-EPI-2C formed grafts most effectively in syngeneic B6 mice compared to other cell lines (10⁷ cells, n=5) (FIG. 25A). In addition, we examined whether the heterogeneity of iPS-EPI cell lines plays an important role in the growth of cells and formation of grafts in vivo. When the same number of cells (10⁷ cells, n=5) were subcutaneously injected into syngeneic B6 mice, the growth rate and ability to form a graft in B6 mice in vivo were significantly reduced in the two single clonal populations (muiPS-EPI-2C clone 1 and muiPS-EPI-2C clone 2) compared to the original, heterogeneous muiPS-EPI-2C cell line (FIG. 25B). Furthermore, we have evaluated the growth ability of cells dissociated from the muiPS-EPI-2C grafts, by injecting those graft-derived cells into the B6 mice (10⁷ cells, n=5) (FIG. 25B). The muiPS-EPI-2C graft-derived cells displayed the significantly improved growth rate and the enhanced ability to form the secondary graft compared to the original muiPS-EPI-2C line.

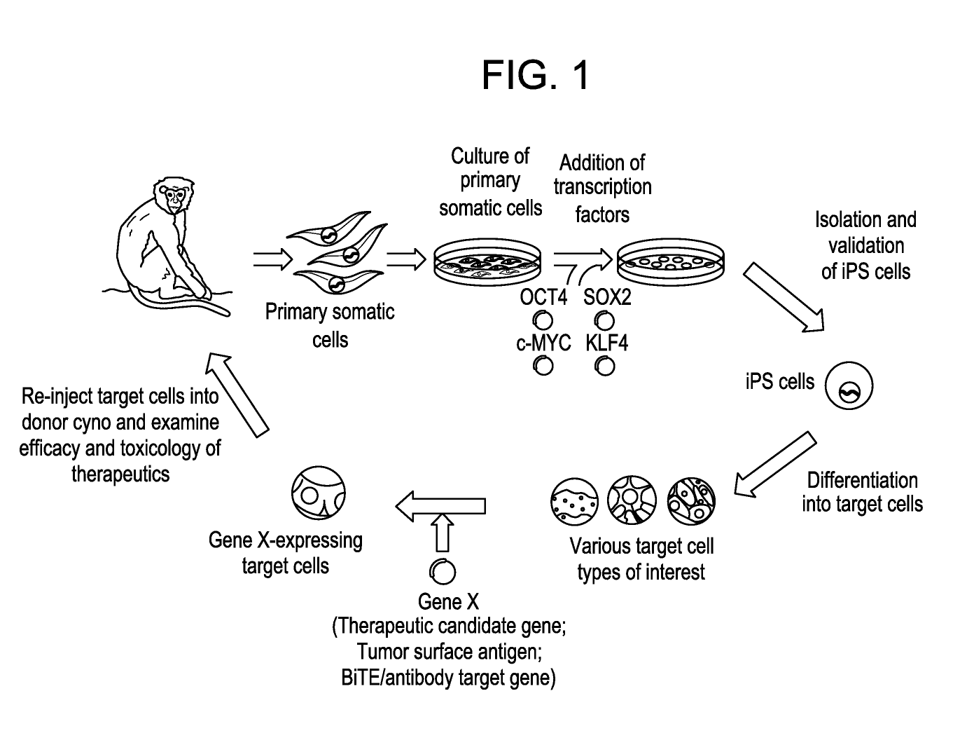

### DETAILED DESCRIPTION OF EMBODIMENTS

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes a plurality of proteins; reference to “a cell” includes populations of a plurality of cells.

“Mammal” refers to any animal classified as a mammal, including humans, domestic and farm animals, and zoo, sports, or pet animals, such as dogs, horses, cats, cows, rodents (e.g., rats, mice, guinea pigs, hamsters), rabbits, pigs, sheep, goats, primates (e.g., monkeys, apes), etc. A “non-human” mammal is a mammal other than a human.

“Non-human primate” or “NHP” means any non-human member of the order Primates, which contains prosimians (including lemurs, lorises, galagos and tarsiers) and, preferably simians (monkeys and apes), for example, baboons (Papio spp.), African green monkeys (Chlorocebus spp.), macaques (e.g., rhesus monkeys (Macaca mulatta), cynomolgus monkeys (Macaca fascicularis)), spider monkeys (Ateles spp.), chimpanzees and bonobos (Pan spp.), gorillas (Gorilla spp.), gibbons (Hylobatidae), and orangutans (Pongo spp.). As noted, cynomolgus monkeys (also known as “cynos”, in singular “cyno”) are macaques (Macaca fascicularis synonym M. cynomolgus).

“Autologous cells” are cells taken from an individual non-human mammal (e.g., a non-human primate, such as a cynomolgus monkey), cultured (or stored), and, optionally, genetically manipulated by recombinant techniques, before being transferred back into the original animal donor. Within the scope of the invention, autologous cells encompass target cell types and effector cell types of interest, as desired.

“Target cell” means a cell that has been reprogrammed (fully or partially), or engineered to mimic a relevant cell type of interest characteristic of a diseased tissue; e.g., by expressing a target antigen for an antibody therapeutic candidate and/or by differentiating in vitro into somatic cells that resemble cells of the relevant diseased tissue. In some embodiments of the invention the target antigen is the product of a tumorigenic gene, an anti-apoptotic gene, an immortalizing gene, or a tumor-related surface antigen. In some embodiments, a target cell is an epithelial-like cell, neuron-like cell, cardiomyocyte, foregut-like cell, midgut-like cell, or hindgut-like cell. In other embodiments, a “target cell” of interest can also be an effector cell type, if desired.

“Effector cell” means an immune effector cell, such as but not limited to these types: a natural killer (NK) cell, macrophage, monocyte, or neutrophil. Within the scope of the invention, an effector cell type of interest can be characteristic of healthy or diseased tissue, as desired.

A “stromal cell” is a connective tissue cell of, or obtained or derived from, connective tissue in an organ or any other body tissue. Examples include stromal cells associated with, or derived from, the uterine mucosa, ovary, prostate, liver, bone marrow, adipose, muscle, and other tissues. A stromal cell from any mammalian source can be used within the scope of the invention, e.g., any of mouse, rat, and rabbit. dog, horse, cat, cow, sheep, pig, monkey (e.g., cyno), ape, or human stromal cells can be used in practicing the method of differentiating non-human primate induced pluripotent stem (iPS) cells, in vitro.

“Definitive endoderm” (“DE”) is a precursor endoderm for organ tissues and can further differentiate into specific organ lineages (foregut, midgut, and hindgut). Within the scope of the present invention, such differentiated gut-like cells can be further useful for the development of target cell types useful in the inventive disease model system, as foregut is the anterior part of primitive gastrointestinal (GI) tract that gives rise to esophagus, trachea, lung, stomach, liver, biliary system, and pancreas, etc.; midgut is the mid-part of the GI tract giving rise to the small intestine; and hindgut is the posterior part of the GI tract that generates the large intestine, including colon, cecum, and rectum, etc., which all can be origins of various tumor types.

“Antibody-dependent cellular cytotoxicity” (“ADCC”) means any immune effector mechanism mediated by antibody-binding that involves killing of target cells by host immune cells. Typically in a mammal, ADCC is a type of immune reaction in which a target cell or microbe is coated with bound antibodies and killed by certain types of white blood cells that express Fc receptors. These white blood cells can include, but are not limited to natural killer (NK) cells, macrophage, monocytes, and/or neutrophils. Most ADCC is mediated by NK cells that express Fc receptor FcγRIII (or CD16) on their surface. The white blood cells bind to the antibodies and release substances that kill the target cells. ADCC is also known as “antibody-dependent cell-mediated cytotoxicity”. (See, Junttila T T et al., Superior in vivo efficacy of afucosylated trastuzumab in the treatment of her2-amplified breast cancer. Cancer Res. 70(11):4481-9 (2010); Varchetta S et al., Elements related to heterogeneity of antibody-dependent cell cytotoxicity in patients under trastuzumab therapy for primary operable breast cancer overexpressing Her2. Cancer Res. 67:11991-9 (2007); Gennari R et al., Pilot study of the mechanism of action of preoperative trastuzumab in patients with primary operable breast tumors overexpressing HER2. Clin Cancer Res. 10:5650-5 (2004); Arina A et al., Cellular liaisons of natural killer lymphocytes in immunology and immunotherapy of cancer. Expert Opin. Biol. Ther. 7(5):599-615(2007); Ottonello L et al., Monoclonal Lym-1 antibody dependent cytolysis by neutrophils exposed to granulocyte-macrophage colony-stimulating factor: intervention of FcγRII (CD32), CD11b-CD18 integrins, and CD66b glycoproteins. Blood 93:3505-3511 (1999); Heijnen I A. et al., Generation of HER-2/neu-specific cytotoxic neutrophils in vivo: efficient arming of neutrophils by combined administration of granulocyte colony-stimulating factor and Fcγ receptor I bispecific antibodies. J Immunol. 159:5629-5639 (1997); Di Carlo E, et al., The intriguing role of polymorphonuclear neutrophils in antitumor reactions. Blood 97:339-345 (2001); Nimmerjahn F and Ravetch J V. Divergent immunoglobulin G subclass activity through selective fc receptor binding. Science 310:1510-2 (2005)). Lymphoid cells can be generated in vitro from bone marrow-derived CD34+CD45+ hematopoietic stem cells. However, the number of cells that can be obtained in this way is limited, especially in the adult mammal. Therefore, the differentiation of human pluripotent stem cells such as embryonic or induced pluripotent stem cells is a valuable source. (See, also, Ni, Z. et al., Human pluripotent stem cells produce natural killer cells that mediate anti-HIV-1 activity by utilizing diverse cellular mechanisms, J Virol 85, 43-50 (2011); Woll, P. S. et al., Human embryonic stem cells differentiate into a homogeneous population of natural killer cells with potent in vivo antitumor activity, Blood 113, 6094-6101 (2009)).

“Administering” means providing entry into the body of, dosing, or otherwise introducing or delivering into, a mammal (including a non-human primate), a substance, such as a therapeutic candidate. Administering the substance can be by any suitable delivery route, such as but not limited to, injection, for example, intramuscularly, intrathecally, epidurally, intravascularly (e.g., intravenously or intraarterially), intraperitoneally or subcutaneously. Sterile solutions can also be administered by intravenous infusion. Any other suitable parenteral or enteral delivery route for delivering the substance into the mammal is encompassed by “administering”.

As used herein, the terms “cell culture medium” and “culture medium” refer to a nutrient solution used for growing mammalian cells in vitro that typically provides at least one component from one or more of the following categories: 1) an energy source, usually in the form of a carbohydrate such as, for example, glucose; 2) one or more of all essential amino acids, and usually the basic set of twenty amino acids plus cysteine; 3) vitamins and/or other organic compounds required at low concentrations; 4) free fatty acids; and 5) trace elements, where trace elements are defined as inorganic compounds or naturally occurring elements that are typically required at very low concentrations, usually in the micromolar range. The nutrient solution may optionally be supplemented with additional components to optimize growth, reprogramming and/or differentiation of cells.

The mammalian cell culture within the present invention is prepared in a medium suitable for the particular cell being cultured. Suitable cell culture media that may be used for culturing a particular cell type would be apparent to one of ordinary skill in the art. Exemplary commercially available media include, for example, Ham's F10 (SIGMA), Minimal Essential Medium (MEM, SIGMA), RPMI-1640 (SIGMA), Dulbecco's Modified Eagle's Medium (DMEM, SIGMA), and DMEM/F12 (Invitrogen). Any of these or other suitable media may be supplemented as necessary with hormones and/or other growth factors (such as but not limited to insulin, transferrin, or epidermal growth factor), salts (such as sodium chloride, calcium, magnesium, and phosphate), buffers (such as HEPES), nucleosides (such as adenosine and thymidine), antibiotics (such as puromycin, neomycin, hygromycin, blasticidin, or Gentamycin™), trace elements (defined as inorganic compounds usually present at final concentrations in the micromolar range) lipids (such as linoleic or other fatty acids) and their suitable carriers, and glucose or an equivalent energy source, and/or modified as described herein to facilitate production of recombinant glycoproteins having low-mannose content. In a particular embodiment, the cell culture medium is serum-free.

When defined medium that is serum-free and/or peptone-free is used, the medium is usually enriched for particular amino acids, vitamins and/or trace elements (see, for example, U.S. Pat. No. 5,122,469 to Mather et al., and U.S. Pat. No. 5,633,162 to Keen et al.). Depending upon the requirements of the particular cell line used or method, culture medium can contain a serum additive such as Fetal Bovine Serum, or a serum replacement. Examples of serum-replacements (for serum-free growth of cells) are TCH™, TM-235™, and TCH™; these products are available commercially from Celox (St. Paul, Minn.), and KOSR (knockout (KO) serum replacement; Invitrogen).

In the methods and compositions of the invention, cells can be grown in serum-free, protein-free, growth factor-free, and/or peptone-free media. The term “serum-free” as applied to media in general includes any mammalian cell culture medium that does not contain serum, such as fetal bovine serum (FBS). The term “insulin-free” as applied to media includes any medium to which no exogenous insulin has been added. By exogenous is meant, in this context, other than that produced by the culturing of the cells themselves. The term “growth-factor free” as applied to media includes any medium to which no exogenous growth factor (e.g., insulin, IGF-1) has been added. The term “peptone-free” as applied to media includes any medium to which no exogenous protein hydrolysates have been added such as, for example, animal and/or plant protein hydrolysates.

Optimally, for purposes of the present invention, the culture medium used is serum-free, or essentially serum-free unless serum is required by the inventive methods or for the growth or maintenance of a particular cell type or cell line. By “serum-free”, it is understood that the concentration of serum in the medium is preferably less than 0.1% (v/v) serum and more preferably less than 0.01% (v/v) serum. By “essentially serum-free” is meant that less than about 2% (v/v) serum is present, more preferably less than about 1% serum is present, still more preferably less than about 0.5% (v/v) serum is present, yet still more preferably less than about 0.1% (v/v) serum is present.

“Culturing” or “incubating” (used interchangeably with respect to the growth, reprogramming, differentiation, and/or maintenance of cells or cell lines) is under conditions of sterility, temperature, pH, atmospheric gas content (e.g., oxygen, carbon dioxide, dinitrogen), humidity, culture container, culture volume, passaging, motion, and other parameters suitable for the intended purpose and conventionally known in the art of mammalian cell culture.

“Polypeptide” and “protein”, or “proteinaceous molecule” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be expressed recombinantly using known protein engineering techniques. In addition, fusion proteins can be derivatized as described herein by well-known organic chemistry techniques. The term “fusion protein” indicates that the protein includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein.

The term “antigen binding protein” (ABP) includes an antibody or antibody fragment, as defined above, a BiTE® (Bi-specific T-cell engager) (e.g., Baeuerle P A, et al., BiTE: Teaching antibodies to engage T-cells for cancer therapy, Curr Opin Mol Ther. 11(1):22-30 (2009)), or a BiKE (Bi-specific killer cell engager) (e.g., Gleason et al., Bispecific and trispecific killer cell engagers directly activate human NK cells through CD16 signaling and induce cytotoxicity and cytokine production, Mol. Cancer Ther. 11(12):1-11 (2012)), and recombinant peptides or other compounds that contain sequences derived from CDRs having the desired antigen-binding properties such that they specifically bind a target antigen of interest. The term “antigen” refers to a molecule or a portion of a molecule capable of being bound by a selective binding agent, such as an antigen binding protein (including, e.g., an antibody or immunological functional fragment thereof), and additionally capable of being used in an animal to produce antibodies capable of binding to that antigen. An antigen may possess one or more epitopes that are capable of interacting with different antigen binding proteins, e.g., antibodies. The term “epitope” is the portion of a molecule that is bound by an antigen binding protein (for example, an antibody). The term includes any determinant capable of specifically binding to an antigen binding protein, such as an antibody or to a T-cell receptor. An epitope can be contiguous or non-contiguous (e.g., in a single-chain polypeptide, amino acid residues that are not contiguous to one another in the polypeptide sequence but that within the context of the molecule are bound by the antigen binding protein). In certain embodiments, epitopes may be mimetic in that they comprise a three dimensional structure that is similar to an epitope used to generate the antigen binding protein, yet comprise none or only some of the amino acid residues found in that epitope used to generate the antigen binding protein. Most often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics. Generally, antibodies specific for a particular target antigen will preferentially recognize an epitope on the target antigen in a complex mixture of proteins and/or macromolecules.

The term “antibody” is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab, Fab′, F(ab′)₂, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes typically have antibody-dependent cellular cytotoxicity (ADCC) activity. Glycosylated and unglycosylated antibodies are included within the term “antibody”.

In general, an antigen binding protein, e.g., an antibody or antibody fragment, “specifically binds” to an antigen when it has a significantly higher binding affinity for, and consequently is capable of distinguishing, that antigen, compared to its affinity for other unrelated proteins, under similar binding assay conditions. Typically, an antigen binding protein is said to “specifically bind” its target antigen when the equilibrium dissociation constant (K_{d}) is ≦10⁻⁸ M. The antibody specifically binds antigen with “high affinity” when the K_{d} is ≦5×10⁻⁹ M, and with “very high affinity” when the K_{d} is ≦5×10⁻¹⁰ M. In one embodiment, the antibodies will bind to a target of interest with a K_{d} of between about 10⁻⁸ M and 10⁻¹⁰ M, and in yet another embodiment the antibodies will bind with a K_{d}≦5×10⁻⁹. In particular embodiments the antigen binding protein, the isolated antigen binding protein specifically binds to a target antigen of interest expressed by a mammalian cell (e.g., CHO, HEK 293, Jurkat), with a K_{d} of 500 pM (5.0×10⁻¹⁰ M) or less, 200 pM (2.0×10⁻¹⁰ M) or less, 150 pM (1.50×10⁻¹⁰ M) or less, 125 pM (1.25×10⁻¹⁰ M) or less, 105 pM (1.05×10⁻¹⁰ M) or less, 50 pM (5.0×10⁻¹¹ M) or less, or 20 pM (2.0×10⁻¹¹ M) or less, as determined by a Kinetic Exclusion Assay, conducted by the method of Rathanaswami et al. (2008) (Rathanaswami et al., High affinity binding measurements of antibodies to cell-surface-expressed antigens, Analytical Biochemistry 373:52-60 (2008; see, e.g., Example 15 herein).

Antigen binding proteins also include peptibodies. The term “peptibody” refers to a molecule comprising an antibody Fc domain attached to at least one peptide. The production of peptibodies is generally described in PCT publication WO 00/24782, published May 4, 2000. Any of these peptides may be linked in tandem (i.e., sequentially), with or without linkers. Peptides containing a cysteinyl residue may be cross-linked with another Cys-containing peptide, either or both of which may be linked to a vehicle. Any peptide having more than one Cys residue may form an intrapeptide disulfide bond, as well. Any of these peptides may be derivatized, for example the carboxyl terminus may be capped with an amino group, cysteines may be cappe, or amino acid residues may substituted by moieties other than amino acid residues (see, e.g., Bhatnagar et al., J. Med. Chem. 39: 3814-9 (1996), and Cuthbertson et al., J. Med. Chem. 40: 2876-82 (1997), which are incorporated by reference herein in their entirety). The peptide sequences may be optimized, analogous to affinity maturation for antibodies, or otherwise altered by alanine scanning or random or directed mutagenesis followed by screening to identify the best binders. Lowman, Ann. Rev. Biophys. Biomol. Struct. 26: 401-24 (1997). Various molecules can be inserted into the antigen binding protein structure, e.g., within the peptide portion itself or between the peptide and vehicle portions of the antigen binding proteins, while retaining the desired activity of antigen binding protein. One can readily insert, for example, molecules such as an Fc domain or fragment thereof, polyethylene glycol or other related molecules such as dextran, a fatty acid, a lipid, a cholesterol group, a small carbohydrate, a peptide, a detectable moiety as described herein (including fluorescent agents, radiolabels such as radioisotopes), an oligosaccharide, oligonucleotide, a polynucleotide, interference (or other) RNA, enzymes, hormones, or the like. Other molecules suitable for insertion in this fashion will be appreciated by those skilled in the art, and are encompassed within the scope of the invention. This includes insertion of, for example, a desired molecule in between two consecutive amino acids, optionally joined by a suitable linker.

The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. Examples of such molecular biological procedures are found in Maniatis et al., Molecular Cloning. A Laboratory Manual. Cold Spring Harbor Laboratory, Cold Spring Harbor, N.Y. (1982). A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including an isotopic label (e.g., ¹²⁵I, ¹⁴C, ¹³C, ³⁵S, ³H, ²H, ¹³N, ¹⁵N, ¹⁸O, ¹⁷O etc.), for ease of quantification or detection, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express a polypeptide (e.g., an oligopeptide or antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. A “fusion gene” contains a coding region that encodes a polypeptide with portions from different proteins that are not naturally found together, or not found naturally together in the same sequence as present in the encoded fusion protein (i.e., a chimeric protein). Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (E.g., Goodey, Andrew R.; et al., Peptide and DNA sequences, U.S. Pat. No. 5,302,697; Weiner et al., Compositions and methods for protein secretion, U.S. Pat. No. 6,022,952 and U.S. Pat. No. 6,335,178; Uemura et al., Protein expression vector and utilization thereof, U.S. Pat. No. 7,029,909; Ruben et al., 27 human secreted proteins, US 2003/0104400 A1).

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial host cells in culture include bacteria (such as Escherichia coli sp.), yeast (such as Saccharomyces sp.) and other fungal cells, insect cells, plant cells, mammalian (including human) cells, e.g., CHO cells and HEK-293 cells. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

A “therapeutic candidate” is any compound, tool compound, combination of compounds, small molecule, polypeptide, peptide, antigen binding protein, antibody or other proteinaceous molecule or biologic, that has or potentially may have therapeutic value in treating, preventing, or mitigating a disease or disorder. The therapeutic candidate is pharmacologically active. The term “pharmacologically active” means that a substance so described is determined to have activity that affects a medical parameter (e.g., blood pressure, blood cell count, cholesterol level, pain perception) or disease state (e.g., cancer, autoimmune disorders, chronic pain). Conversely, the term “pharmacologically inactive” means that no activity affecting a medical parameter or disease state can be determined for that substance. Thus, pharmacologically active molecules, comprise agonistic or mimetic and antagonistic molecules as defined below.

The terms “-mimetic peptide,” “peptide mimetic,” and “-agonist peptide” refer to a peptide or protein having biological activity comparable to a naturally occurring protein of interest. These terms further include peptides that indirectly mimic the activity of a naturally occurring peptide molecule, such as by potentiating the effects of the naturally occurring molecule.

An “agonist” is a molecule that binds to a receptor of interest and triggers a response by the cell bearing the receptor. Agonists often mimic the action of a naturally occurring substance. An “inverse agonist” causes an action opposite to that of the agonist.

The term “antagonist” and “inhibitor” refer to a molecule that blocks or in some way interferes with the biological activity of a receptor of interest, or has biological activity comparable to a known antagonist or inhibitor of a receptor of interest (such as, but not limited to, an ion channel or a G-Protein Coupled Receptor (GPCR)).

A “tool compound” is any small molecule, peptide, antigen binding protein, antibody or other proteinaceous molecule, employed as a reagent used in an experiment, as a control, or as a pharmacologically active surrogate compound in place of a therapeutic candidate.

A “transgenic-knock-in” or “knock-in” construct expresses a foreign gene in the locus of the endogenous host gene; such as a human gene in the non-human locus of the equivalent gene. In addition, a readily detectable and/or assayable marker gene, such as a luciferase gene or antibody resistance gene, can be incorporated into the expression construct whose expression or presence in the genome can easily be detected. The marker gene is usually operably linked to its own promoter or to another strong promoter from any source that will be active or can easily be activated in the cell into which it is inserted; however, the marker gene need not have its own promoter attached as it may be transcribed using the promoter of the gene of interest to be expressed (or suppressed, in the case of a knock-out construct; see, below). In addition, the marker gene will normally have a polyA sequence attached to the 3′ end of the gene; this sequence serves to terminate transcription of the gene. Preferred marker genes are luciferase, beta-gal (beta-galactosidase), or any antibiotic resistance gene such as neo (the neomycin resistance gene).

The term “knockout construct” refers to a nucleic acid sequence that is designed to decrease or suppress expression of a protein encoded by endogenous DNA sequences in a cell. The nucleic acid sequence used as the knockout construct is typically comprised of (1) DNA from some portion of the gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise 1) a full or partial sequence of one or more exons and/or introns of the gene to be suppressed, 2) a full or partial promoter sequence of the gene to be suppressed, or 3) combinations thereof.

A knockout or knock-in construct can be inserted into an embryonic stem cell (ES cell) and is integrated into the ES cell genomic DNA, usually by the process of homologous recombination. This ES cell is then injected into, and integrates with, the developing embryo. Alternatively, a knock-out or knock-in construct can be incorporated into an iPS cell.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene.

The term “transgene” refers to an isolated nucleotide sequence, originating in a different species from the host, that may be inserted into one or more cells of a mammal or mammalian embryo. The transgene optionally may be operably linked to other genetic elements (such as a promoter, poly A sequence and the like) that may serve to modulate, either directly, or indirectly in conjunction with the cellular machinery, the transcription and/or expression of the transgene. Alternatively or additionally, the transgene may be linked to nucleotide sequences that aid in integration of the transgene into the chromosomal DNA of the mammalian cell or embryo nucleus (as for example, in homologous recombination). The transgene may be comprised of a nucleotide sequence that is either homologous or heterologous to a particular nucleotide sequence in the mammal's endogenous genetic material, or is a hybrid sequence (i.e. one or more portions of the transgene are homologous, and one or more portions are heterologous to the mammal's genetic material). The transgene nucleotide sequence may encode a polypeptide or a variant of a polypeptide, found endogenously in the mammal, it may encode a polypeptide not naturally occurring in the mammal (i.e. an exogenous polypeptide), or it may encode a hybrid of endogenous and exogenous polypeptides. Where the transgene is operably linked to a promoter, the promoter may be homologous or heterologous to the mammal and/or to the transgene. Alternatively, the promoter may be a hybrid of endogenous and exogenous promoter elements (enhancers, silencers, suppressors, and the like).

The term “progeny” refers to any and all future generations derived and descending from a particular cell or mammal.

“DAPI” or 4′,6-diamidino-2-phenylindole is a fluorescent stain that binds strongly to A-T rich regions in DNA. It is used extensively in fluorescence microscopy. DAPI can pass through an intact cell membrane therefore it can be used to stain both live and fixed cells, though it passes through the membrane less efficiently in live cells and therefore the effectiveness of the stain is lower for live cells.

“Reprogramming” refers to a manipulation (such as but not limited to exposing a cell to certain defined growth or transcription factors) that changes the developmental fate of the cell in a way that can be detected by one or more changes in gene expression, such as changes in biomarkers (e.g., membrane, cytoplasmic or nuclear biomarkers), morphology, and/or the physiological role of the cell. Such a manipulated cell, or its subsequent generations, is a “reprogrammed” cell with changes in morphology (in vivo or in vitro) and/or physiological role (in vivo or in vitro), compared to before reprogramming. Examples of reprogramming include turning one cell type into another cell type, reprogramming adult somatic cells into induced pluripotent stem (iPS) cells and lineage conversion. Reprogramming may induce the remodeling of a cell's epigenetic markers, for example, through mechanisms thought to involve polycomb proteins, demethylation and/or hypermethylation of genes or promoters.

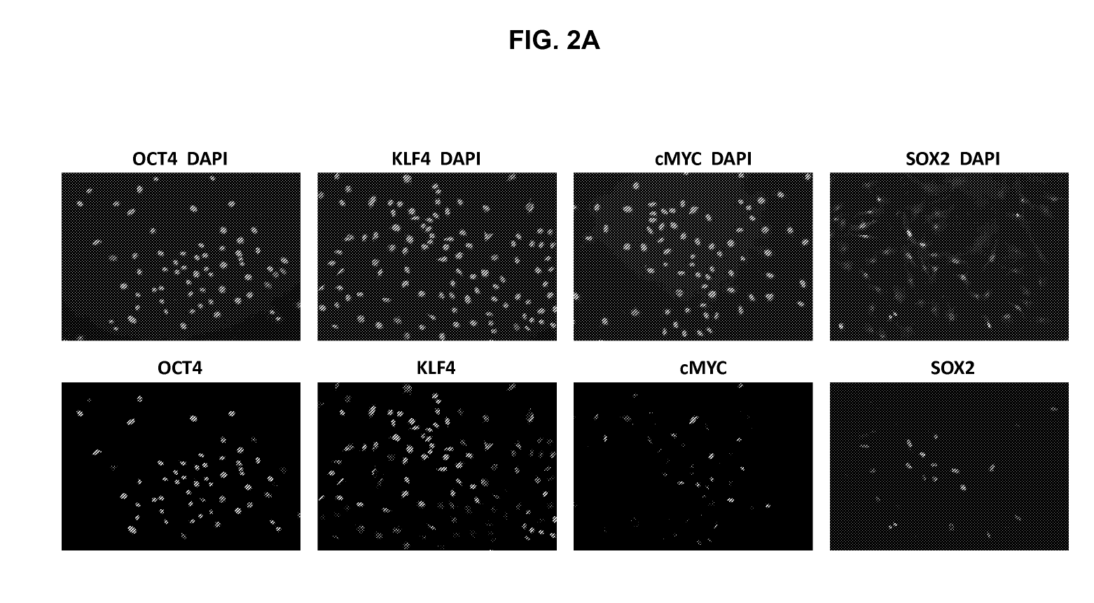

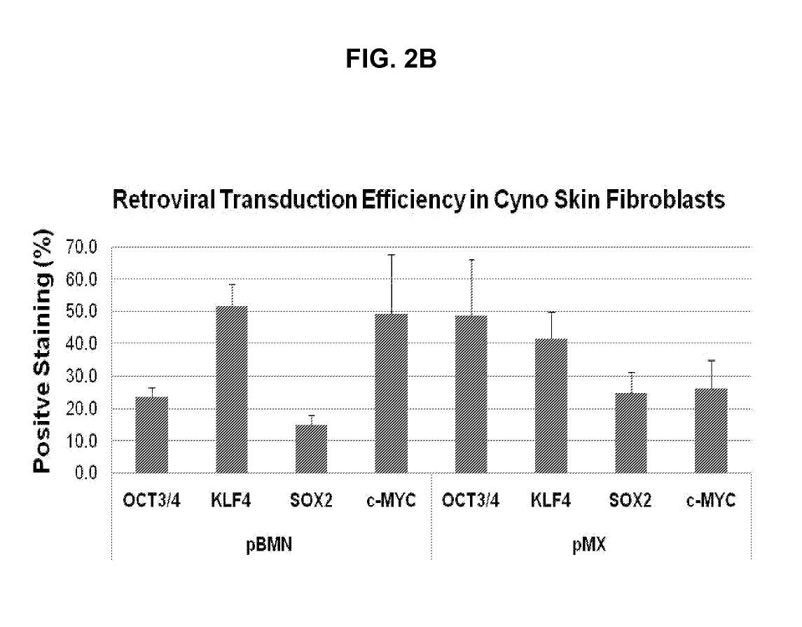

Reprogramming of adult somatic cells into a pluripotent (embryonic stem cell-like) state can be induced through ectopic expression of transcription factors, e.g., OCT4, SOX2, KLF4, and c-MYC (see, FIG. 2A-B). These reprogrammed, pluripotent iPS cells can differentiate to form all of the cell types in the body. This iPS cell technology provides invaluable resources in sufficient amounts, without the use of embryonic material, for diverse therapeutic application, including autologous transplantation and establishing histocompatible stem cell banks, patient-specific disease modeling, drug screening and regenerative medicine. To create an autologous NHP model, cyno iPS cells were generated by isolating cyno skin fibroblasts from individual cyno monkeys and reprogramming them. These autologous cyno iPS cells were further differentiated into multiple target cells to generate autologous target cells.

Induction of pluripotency by reprogramming differentiated somatic cells was originally achieved by Yamanaka group from mouse somatic cells in 2006 and from human somatic cells in 2007 (Takahashi, K. et al., Induction of pluripotent stem cells from adult human fibroblasts by defined factors, Cell 131, 861-872 (2007); Takahashi, K., and Yamanaka, S. Induction of pluripotent stem cells from mouse embryonic and adult fibroblast cultures by defined factors, Cell 126, 663-676 (2006)). Differentiation of embryonic stem (ES) or induced pluripotent stem (iPS) cells has been demonstrated into the following cell types, including definitive endoderm (“DE”), foregut endoderm, intestinal tissue (hindgut), pancreatic insulin-producing cells, hepatocytes, neurons, cardiac myocytes, endothelial cells, hematopoietic progenitors, T cells, NKT cells, and Natural Killer (NK) cells. (Basma, H. et al., Differentiation and transplantation of human embryonic stem cell-derived hepatocytes, Gastroenterology 136, 990-999 (2009); D'Amour, K. A. et al., Efficient differentiation of human embryonic stem cells to definitive endoderm, Nat Biotechnol 23, 1534-1541 (2005); Deleidi, M. et al., Development of histocompatible primate-induced pluripotent stem cells for neural transplantation, Stem Cells 29, 1052-1063 (July 2011; article first published online: 29 Jun. 2011, DOI: 10.1002/stem.662); Dimos, J. T. et al., Induced pluripotent stem cells generated from patients with ALS can be differentiated into motor neurons, Science 321, 1218-1221 (2008); Green, M. D. et al., Generation of anterior foregut endoderm from human embryonic and induced pluripotent stem cells, Nat Biotechnol 29, 267-272. (March 2011); Jiang, J. et al., Generation of insulin-producing islet-like clusters from human embryonic stem cells, Stem Cells 25, 1940-1953 (2007); Mauritz, C. et al., Generation of functional murine cardiac myocytes from induced pluripotent stem cells, Circulation 118, 507-517 (2008); Ni, Z. et al., Human pluripotent stem cells produce natural killer cells that mediate anti-HIV-1 activity by utilizing diverse cellular mechanisms, J Virol 85, 43-50 (2011); Spence, J. R. et al., Directed differentiation of human pluripotent stem cells into intestinal tissue in vitro, Nature 470:105-109 (February 2011; Epub 2010 Dec. 12)).

Somatic cell nuclear transfer technique has demonstrated that somatic nuclei can be reprogrammed to a primitive state to create new (clone) embryos. Recently, it was shown that ectopic expression of key transcription factors that are known to be important for maintaining pluripotent stem cells can reprogram somatic cells into a pluripotent state (thereby, generating iPS cells). In addition, a recent study showed that one somatic cell type can directly be converted (i.e., “reprogrammed”) into another cell type by the expression and/or presence of essential transcription factors, without going through the pluripotent state. (See, Konrad Hochedlinger and Kathrin Plath. Epigenetic reprogramming and induced pluripotency. Development 136:509-523 (2009); Vierbuchen, T et al., Direct conversion of fibroblasts to functional neurons by defined factors, Nature 25, 1035-41 (2010)).

Selection of Transgene(s).

Typically, the transgene(s) useful in the present invention for reprogramming iPS cells will be a nucleotide sequence encoding a polypeptide of interest, e.g., a polypeptide involved in the nervous system, an immune response, hematopoiesis, inflammation, cell growth and proliferation, cell lineage differentiation, and/or the stress response. Included within the scope of this invention is the insertion of one, two, or more transgenes into an iPS cell.

Where more than one transgene is used in this invention, the transgenes may be prepared and inserted individually, or may be generated together as one construct for insertion. The transgenes may be homologous or heterologous to both the promoter selected to drive expression of each transgene and/or to the mammal. Further, the transgene may be a full length cDNA or genomic DNA sequence, or any fragment, subunit or mutant thereof that has at least some biological activity i.e., exhibits an effect at any level (biochemical, cellular and/or morphological) that is not readily observed in a wild type, non-transgenic mammal of the same species. Optionally, the transgene may be a hybrid nucleotide sequence, i.e., one constructed from homologous and/or heterologous cDNA and/or genomic DNA fragments. The transgene may also optionally be a mutant of one or more naturally occurring cDNA and/or genomic sequences, or an allelic variant thereof.

Each transgene may be isolated and obtained in suitable quantity using one or more methods that are well known in the art. These methods and others useful for isolating a transgene are set forth, for example, in Sambrook et al. (Molecular Cloning: A Laboratory Manual, Cold Spring Harbor Laboratory Press, Cold Spring Harbor, N.Y. [1989]) and in Berger and Kimmel (Methods in Enzymology: Guide to Molecular Cloning Techniques, vol. 152, Academic Press, Inc., San Diego, Calif. [1987]).

Where the nucleotide sequence of each transgene is known, the transgene may be synthesized, in whole or in part, using chemical synthesis methods such as those described in Engels et al. (Angew. Chem. Int. Ed. Engl., 28:716-734 [1989]). These methods include, inter alia, the phosphotriester, phosphoramidite and H-phosphonate methods of nucleic acid synthesis. Alternatively, the transgene may be obtained by screening an appropriate cDNA or genomic library using one or more nucleic acid probes (oligonucleotides, cDNA or genomic DNA fragments with an acceptable level of homology to the transgene to be cloned, and the like) that will hybridize selectively with the transgene DNA. Another suitable method for obtaining a transgene is the polymerase chain reaction (PCR). However, successful use of this method requires that enough information about the nucleotide sequence of the transgene be available so as to design suitable oligonucleotide primers useful for amplification of the appropriate nucleotide sequence.

Where the method of choice requires the use of oligonucleotide primers or probes (e.g. PCR, cDNA or genomic library screening), the oligonucleotide sequences selected as probes or primers should be of adequate length and sufficiently unambiguous so as to minimize the amount of non-specific binding that will occur during library screening or PCR. The actual sequence of the probes or primers is usually based on conserved or highly homologous sequences or regions from the same or a similar gene from another organism. Optionally, the probes or primers can be degenerate.

In cases where only the amino acid sequence of the transgene is known, a probable and functional nucleic acid sequence may be inferred for the transgene using known and preferred codons for each amino acid residue. This sequence can then be chemically synthesized.

This invention encompasses the use of transgene mutant sequences. A mutant transgene is a transgene containing one or more nucleotide substitutions, deletions, and/or insertions as compared to the wild type sequence. The nucleotide substitution, deletion, and/or insertion can give rise to a gene product (i.e., protein) that is different in its amino acid sequence from the wild type amino acid sequence. Preparation of such mutants is well known in the art, and is described for example in Wells et al. (Gene, 34:315 [1985]), and in Sambrook et al, supra.

Selection of Regulatory Elements.

Transgenes are typically operably linked to promoters, where a promoter is selected to regulate expression of each transgene in a particular manner.

Where more than one transgene is to be used, each transgene may be regulated by the same or by a different promoter. The selected promoters may be homologous (i.e., from the same species as the mammal to be transfected with the transgene) or heterologous (i.e., from a source other than the species of the mammal to be transfected with the transgene). As such, the source of each promoter may be from any unicellular, prokaryotic or eukaryotic organism, or any vertebrate or invertebrate organism.

Selection of Other Vector Components

In addition to the transgene and the promoter, the vectors useful for preparing the transgenes of this invention typically contain one or more other elements useful for (1) optimal expression of transgene in the mammal into which the transgene is inserted, and (2) amplification of the vector in bacterial or mammalian host cells. Each of these elements will be positioned appropriately in the vector with respect to each other element so as to maximize their respective activities. Such positioning is well known to the ordinary skilled artisan. The following elements may be optionally included in the vector as appropriate.

i. Signal Sequence Element

For those embodiments of the invention where the polypeptide encoded by the transgene is to be secreted, a small polypeptide termed signal sequence is frequently present to direct the polypeptide encoded by the transgene out of the cell where it is synthesized. Typically, the signal sequence is positioned in the coding region of the transgene towards or at the 5′ end of the coding region. Many signal sequences have been identified, and any of them that are functional and thus compatible with the transgenic tissue may be used in conjunction with the transgene. Therefore, the nucleotide sequence encoding the signal sequence may be homologous or heterologous to the transgene, and may be homologous or heterologous to the transgenic mammal. Additionally, the nucleotide sequence encoding the signal sequence may be chemically synthesized using methods set forth above. However, for purposes herein, preferred signal sequences are those that occur naturally with the transgene (i.e., are homologous to the transgene).

ii. Membrane Anchoring Domain Element

In some cases, it may be desirable to have a transgene expressed on the surface of a particular intracellular membrane or on the plasma membrane. Naturally occurring membrane proteins contain, as part of the polypeptide, a stretch of amino acids that serve to anchor the protein to the membrane. However, for proteins that are not naturally found on the membrane, such a stretch of amino acids may be added to confer this feature. Frequently, the anchor domain will be an internal portion of the polypeptide sequence and thus the nucleotide sequence encoding it will be engineered into an internal region of the transgene nucleotide sequence. However, in other cases, the nucleotide sequence encoding the anchor domain may be attached to the 5′ or 3′ end of the transgene nucleotide sequence. Here, the nucleotide sequence encoding the anchor domain may first be placed into the vector in the appropriate position as a separate component from the nucleotide sequence encoding the transgene. As for the signal sequence, the anchor domain may be from any source and thus may be homologous or heterologous with respect to both the transgene and the transgenic mammal. Alternatively, the anchor domain may be chemically synthesized using methods set forth above.

iii. Origin of Replication Element

This component is typically a part of prokaryotic expression vectors purchased commercially, and aids in the amplification of the vector in a host cell. If the vector of choice does not contain an origin of replication site, one may be chemically synthesized based on a known sequence, and ligated into the vector.

iv. Transcription Termination Element

This element, also known as the polyadenylation or polyA sequence, is typically located 3′ to the transgene nucleotide sequence in the vector, and serves to terminate transcription of the transgene. While the nucleotide sequence encoding this element is easily cloned from a library or even purchased commercially as part of a vector, it can also be readily synthesized using methods for nucleotide sequence synthesis such as those described above.

v. Intron Element

In many cases, transcription of the transgene is increased by the presence of one intron or more than one intron (linked by exons) on the cloning vector. The intron(s) may be naturally occurring within the transgene nucleotide sequence, especially where the transgene is a full length or a fragment of a genomic DNA sequence. Where the intron(s) is not naturally occurring within the nucleotide sequence (as for most cDNAs), the intron(s) may be obtained from another source. The intron(s) may be homologous or heterologous to the transgene and/or to the transgenic mammal. The position of the intron with respect to the promoter and the transgene is important, as the intron must be transcribed to be effective. As such, where the transgene is a cDNA sequence, the preferred position for the intron(s) is 3′ to the transcription start site, and 5′ to the polyA transcription termination sequence. Preferably for cDNA transgenes, the intron will be located on one side or the other (i.e., 5′ or 3′) of the transgene nucleotide sequence such that it does not interrupt the transgene nucleotide sequence. Any intron from any source, including any viral, prokaryotic and eukaryotic (plant or animal) organisms, may be used to practice this invention, provided that it is compatible with the host cell(s) into which it is inserted. Also included herein are synthetic introns. Optionally, more than one intron may be used in the vector. A preferred set of introns and exons is the human growth hormone (hGH) DNA sequence.

vi. Selectable Marker(s) Element

Selectable marker genes encode polypeptides necessary for the survival and growth of transfected cells grown in a selective culture medium. Typical selection marker genes encode proteins that (a) confer resistance to antibiotics or other toxins, e.g., ampicillin, tetracycline, or kanomycin for prokaryotic host cells, and neomycin, hygromycin, or methotrexate for mammalian cells; (b) complement auxotrophic deficiencies of the cell; or (c) supply critical nutrients not available from complex media, e.g., the gene encoding D-alanine racemase for cultures of Bacilli.

All of the elements set forth above, as well as others useful in this invention, are well known to the skilled artisan and are described, for example, in Sambrook et al. (Molecular Cloning: A Laboratory Manual, Cold Spring Harbor Laboratory Press, Cold Spring Harbor, N.Y. [1989]) and Berger et al., eds. (Guide to Molecular Cloning Techniques, Academic Press, Inc., San Diego, Calif. [1987]).

Construction of Cloning Vectors

The cloning vectors most useful for amplification of transgene cassettes useful in preparing the transgenic mammals of this invention are those that are compatible with prokaryotic cell hosts. However, eukaryotic cell hosts, and vectors compatible with these cells, are within the scope of the invention.

In certain cases, some of the various elements to be contained on the cloning vector may be already present in commercially available cloning or amplification vectors such as pUC18, pUC19, pBR322, the pGEM vectors (Promega Corp, Madison, Wis.), the pBluescript® vectors such as pBIISK+/−(Stratagene Corp., La Jolla, Calif.), and the like, all of which are suitable for prokaryotic cell hosts. In this case it is necessary to only insert the transgene(s) into the vector.

However, where one or more of the elements to be used are not already present on the cloning or amplification vector, they may be individually obtained and ligated into the vector. Methods used for obtaining each of the elements and ligating them are well known to the skilled artisan and are comparable to the methods set forth above for obtaining a transgene (i.e., synthesis of the DNA, library screening, and the like).

Vectors used for cloning or amplification of the transgene(s) nucleotide sequences and/or for transfection of the mammalian embryos are constructed using methods well known in the art. Such methods include, for example, the standard techniques of restriction endonuclease digestion, ligation, agarose and acrylamide gel purification of DNA and/or RNA, column chromatography purification of DNA and/or RNA, phenol/chloroform extraction of DNA, DNA sequencing, polymerase chain reaction amplification, and the like, as set forth in Sambrook et al., supra.

The final vector used to practice this invention is typically constructed from a starting cloning or amplification vector such as a commercially available vector. This vector may or may not contain some of the elements to be included in the completed vector. If none of the desired elements are present in the starting vector, each element may be individually ligated into the vector by cutting the vector with the appropriate restriction endonuclease(s) such that the ends of the element to be ligated in and the ends of the vector are compatible for ligation. In some cases, it may be necessary to “blunt” the ends to be ligated together in order to obtain a satisfactory ligation. Blunting is accomplished by first filling in “sticky ends” using Klenow DNA polymerase or T4 DNA polymerase in the presence of all four nucleotides. This procedure is well known in the art and is described for example in Sambrook et al., supra.

Alternatively, two or more of the elements to be inserted into the vector may first be ligated together (if they are to be positioned adjacent to each other) and then ligated into the vector.

One other method for constructing the vector is to conduct all ligations of the various elements simultaneously in one reaction mixture. Here, many nonsense or nonfunctional vectors will be generated due to improper ligation or insertion of the elements, however the functional vector may be identified and selected by restriction endonuclease digestion.

After the vector has been constructed, it may be transfected into a prokaryotic host cell for amplification. Cells typically used for amplification are E coli DH5-alpha (Gibco/BRL, Grand Island, N.Y.) and other E. coli strains with characteristics similar to DH5-alpha.

Where mammalian host cells are used, cell lines such as Chinese hamster ovary (CHO cells; Urlab et al., Proc. Natl. Acad. Sci USA, 77:4216 [1980])) and human embryonic kidney cell line 293 (Graham et al., J. Gen. Virol., 36:59 [1977]), as well as other lines, are suitable.

Transfection of the vector into the selected host cell line for amplification is accomplished using such methods as calcium phosphate, electroporation, microinjection, lipofection or DEAE-dextran. The method selected will in part be a function of the type of host cell to be transfected. These methods and other suitable methods are well known to the skilled artisan, and are set forth in Sambrook et al., supra.

After culturing the cells long enough for the vector to be sufficiently amplified (usually overnight for E. coli cells), the vector (often termed plasmid at this stage) is isolated from the cells and purified. Typically, the cells are lysed and the plasmid is extracted from other cell contents. Methods suitable for plasmid purification include inter alia, the alkaline lysis mini-prep method (Sambrook et al., supra).

Preparation of Plasmid for Insertion

Typically, the plasmid containing the transgene is linearized, and portions of it removed using a selected restriction endonuclease prior to insertion into the embryo. In some cases, it may be preferable to isolate the transgene, promoter, and regulatory elements as a linear fragment from the other portions of the vector, thereby injecting only a linear nucleotide sequence containing the transgene, promoter, intron (if one is to be used), enhancer, polyA sequence, and optionally a signal sequence or membrane anchoring domain into the embryo. This may be accomplished by cutting the plasmid so as to remove the nucleic acid sequence region containing these elements, and purifying this region using agarose gel electrophoresis or other suitable purification methods.

Therapeutic Candidate Compounds

Production of Antibodies

Polyclonal Antibodies.

Polyclonal antibodies are typically raised in animals by multiple subcutaneous (sc) or intraperitoneal (ip) injections of the relevant antigen and an adjuvant. Alternatively, antigen may be injected directly into the animal's lymph node (see Kilpatrick et al., Hybridoma, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates, or derivatives by combining, e.g., 100 μg of the protein or conjugate (for mice) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies.

Monoclonal antibodies can be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. For example, monoclonal antibodies can be made using the hybridoma method first described by Kohler et al., Nature, 256:495 (1975), or can be made by recombinant DNA methods (e.g., Cabilly et al., Methods of producing immunoglobulins, vectors and transformed host cells for use therein, U.S. Pat. No. 6,331,415), including methods, such as the “split DHFR” method, that facilitate the generally equimolar production of light and heavy chains, optionally using mammalian cell lines (e.g., CHO cells) that can glycosylate the antibody (See, e.g., Page, Antibody production, EP0481790 A2 and U.S. Pat. No. 5,545,403).

In the hybridoma method, a mouse or other appropriate host mammal, such as rats, hamster or macaque monkey, is immunized as herein described to elicit lymphocytes that produce or are capable of producing antibodies that will specifically bind to the protein used for immunization. Alternatively, lymphocytes can be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells, once prepared, are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by BIAcore® or Scatchard analysis (Munson et al., Anal. Biochem., 107:220 (1980); Fischer et al., A peptide-immunoglobulin-conjugate, WO 2007/045463 A1, Example 10, which is incorporated herein by reference in its entirety).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

Hybridomas or mAbs may be further screened to identify mAbs with particular properties, such as binding affinity with a particular antigen or target. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, affinity chromatography, or any other suitable purification technique known in the art.

Recombinant Production of Antibodies and Other Polypeptides.

Relevant amino acid sequences from an immunoglobulin or polypeptide of interest may be determined by direct protein sequencing, and suitable encoding nucleotide sequences can be designed according to a universal codon table. Alternatively, genomic or cDNA encoding the monoclonal antibodies may be isolated and sequenced from cells producing such antibodies using conventional procedures (e.g., by using oligonucleotide probes that are capable of binding specifically to genes encoding the heavy and light chains of the monoclonal antibodies). Relevant DNA sequences can be determined by direct nucleic acid sequencing.

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+ mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

The sequence encoding an entire variable region of the immunoglobulin polypeptide may be determined; however, it will sometimes be adequate to sequence only a portion of a variable region, for example, the CDR-encoding portion. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, one of skill will readily be able to determine, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence (that may, for example, direct secretion of the antibody; e.g., ATGGACATGAGGGTGCCCGCTCAGCTCCTGGGGCTCCTGCTGCTGTGGCTG AGAGGTGCGCGCTGT// SEQ ID NO:1, which encodes the VK-1 signal peptide sequence MDMRVPAQLLGLLLLWLRGARC// SEQ ID NO:2), an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

Cell, cell line, and cell culture are often used interchangeably and all such designations herein include progeny. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus such as B. subtilis and B. licheniformis, Pseudomonas, and Streptomyces. Eukaryotic microbes such as filamentous fungi or yeast are suitable cloning or expression hosts for recombinant polypeptides or antibodies. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Host cells for the expression of glycosylated antibodies can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of polypeptides (including antibody) from such cells has become routine procedure. Examples of useful mammalian host cell lines are Chinese hamster ovary cells, including CHOK1 cells (ATCC CCL61), DXB-11, DG-44, and Chinese hamster ovary cells/-DHFR (CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68 (1982)); MRC 5 cells or FS4 cells; or mammalian myeloma cells.

Host cells are transformed or transfected with the above-described nucleic acids or vectors for production of polypeptides (including antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides, such as antibodies.

The host cells used to produce the polypeptides useful in the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the recombinant polypeptide can be produced intracellularly, in the periplasmic space, or directly secreted into the medium. If the polypeptide, such as an antibody, is produced intracellularly, as a first step, the particulate debris, either host cells or lysed fragments, is removed, for example, by centrifugation or ultrafiltration.

An antibody or antibody fragment) can be purified using, for example, hydroxylapatite chromatography, cation or anion exchange chromatography, or preferably affinity chromatography, using the antigen of interest or protein A or protein G as an affinity ligand. Protein A can be used to purify proteins that include polypeptides are based on human γ1, γ2, or γ4 heavy chains (Lindmark et al., J. Immunol. Meth. 62: 1-13 (1983)). Protein G is recommended for all mouse isotypes and for human γ3 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a C_{H} 3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the antibody to be recovered.

Chimeric, Humanized, Human Engineered™, Xenomouse® Monoclonal Antibodies.

Chimeric monoclonal antibodies, in which the variable Ig domains of a rodent monoclonal antibody are fused to human constant Ig domains, can be generated using standard procedures known in the art (See Morrison, S. L., et al. (1984) Chimeric Human Antibody Molecules; Mouse Antigen Binding Domains with Human Constant Region Domains, Proc. Natl. Acad. Sci. USA 81, 6841-6855; and, Boulianne, G. L., et al, Nature 312, 643-646. (1984)). A number of techniques have been described for humanizing or modifying antibody sequence to be more human-like, for example, by (1) grafting the non-human complementarity determining regions (CDRs) onto a human framework and constant region (a process referred to in the art as humanizing through “CDR grafting”) or (2) transplanting the entire non-human variable domains, but “cloaking” them with a human-like surface by replacement of surface residues (a process referred to in the art as “veneering”) or (3) modifying selected non-human amino acid residues to be more human, based on each residue's likelihood of participating in antigen-binding or antibody structure and its likelihood for immunogenicity. See, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference in its entirety.

A number of techniques have been described for humanizing or modifying antibody sequence to be more human-like, for example, by (1) grafting the non-human complementarity determining regions (CDRs) onto a human framework and constant region (a process referred to in the art as humanizing through “CDR grafting”) or (2) transplanting the entire non-human variable domains, but “cloaking” them with a human-like surface by replacement of surface residues (a process referred to in the art as “veneering”) or (3) modifying selected non-human amino acid residues to be more human, based on each residue's likelihood of participating in antigen-binding or antibody structure and its likelihood for immunogenicity. See, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference in its entirety.

Antibodies can also be produced using transgenic animals that have no endogenous immunoglobulin production and are engineered to contain human immunoglobulin loci. (See, e.g., Mendez et al., Nat. Genet. 15:146-156 (1997)) For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein. See also Jakobovits et al., Proc. Natl. Acad. Sci. USA, 90:2551 (1993); Jakobovits et al., Nature, 362:255-258 (1993); Bruggermann et al., Year in Immuno., 7:33 (1993); Mendez et al., Nat. Genet. 15:146-156 (1997); and U.S. Pat. No. 5,591,669, U.S. Pat. No. 5,589,369, U.S. Pat. No. 5,545,807; and U.S. Patent Application No. 20020199213. U.S. Patent Application No. and 20030092125 describes methods for biasing the immune response of an animal to the desired epitope. Human antibodies may also be generated by in vitro activated B cells (see U.S. Pat. Nos. 5,567,610 and 5,229,275).

Antibody Production by Phage Display Techniques

The development of technologies for making repertoires of recombinant human antibody genes, and the display of the encoded antibody fragments on the surface of filamentous bacteriophage, has provided another means for generating human-derived antibodies. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference in its entirety. The antibodies produced by phage technology are usually produced as antigen binding fragments, e.g. Fv or Fab fragments, in bacteria and thus lack effector functions. Effector functions can be introduced by one of two strategies: The fragments can be engineered either into complete antibodies for expression in mammalian cells, or into bispecific antibody fragments with a second binding site capable of triggering an effector function.

Typically, the Fd fragment (V_{H}-C_{H}1) and light chain (V_{L}-C_{L}) of antibodies are separately cloned by PCR and recombined randomly in combinatorial phage display libraries, which can then be selected for binding to a particular antigen. The antibody fragments are expressed on the phage surface, and selection of Fv or Fab (and therefore the phage containing the DNA encoding the antibody fragment) by antigen binding is accomplished through several rounds of antigen binding and re-amplification, a procedure termed panning Antibody fragments specific for the antigen are enriched and finally isolated.

Phage display techniques can also be used in an approach for the humanization of rodent monoclonal antibodies, called “guided selection” (see Jespers, L. S., et al., Bio/Technology 12, 899-903 (1994)). For this, the Fd fragment of the mouse monoclonal antibody can be displayed in combination with a human light chain library, and the resulting hybrid Fab library may then be selected with antigen. The mouse Fd fragment thereby provides a template to guide the selection. Subsequently, the selected human light chains are combined with a human Fd fragment library. Selection of the resulting library yields entirely human Fab.

A variety of procedures have been described for deriving human antibodies from phage-display libraries (See, for example, Hoogenboom et al., J. Mol. Biol., 227:381 (1991); Marks et al., J. Mol. Biol, 222:581-597 (1991); U.S. Pat. Nos. 5,565,332 and 5,573,905; Clackson, T., and Wells, J. A., TIBTECH 12, 173-184 (1994)). In particular, in vitro selection and evolution of antibodies derived from phage display libraries has become a powerful tool (See Burton, D. R., and Barbas III, C. F., Adv. Immunol. 57, 191-280 (1994); and, Winter, G., et al., Annu. Rev. Immunol. 12, 433-455 (1994); U.S. patent application no. 20020004215 and WO92/01047; U.S. patent application no. 20030190317 published Oct. 9, 2003 and U.S. Pat. No. 6,054,287; U.S. Pat. No. 5,877,293.Watkins, “Screening of Phage-Expressed Antibody Libraries by Capture Lift,” Methods in Molecular Biology, Antibody Phage Display: Methods and Protocols 178: 187-193, and U.S. Patent Application Publication No. 20030044772 published Mar. 6, 2003 describes methods for screening phage-expressed antibody libraries or other binding molecules by capture lift, a method involving immobilization of the candidate binding molecules on a solid support.

The invention will be more fully understood by reference to the following examples. These examples are not to be construed in any way as limiting the scope of this invention.

### EXAMPLES

### Example 1

### Materials and Methods

Fibroblast Cell Culture and Animals.

Skin biopsies were obtained from ten female Chinese cynomolgus macaques (˜3 years old; Charles River Laboratories, Reno, Nev.) and cynomolgus macaques (SNBL; Everett, Wash.). The cyno skin fibroblasts were isolated from dorsal skins of cyno monkeys and passaged multiple times (˜4 passages). The skin biopsies were minced with a sterile blade in DMEM, pH 7.4, containing 2 mg/ml collagenase IV (Invitrogen, #17104-019) in DMEM and 1 U/ml dispase (Invitrogen), and then were incubated at 37° C. for 2 hours. The skin cells were collected, filtered through the 70 μm strainer and washed. The resulting skin fibroblasts were cultured at 37° C. in DMEM, pH 7.4, containing 10% (v/v) fetal bovine serum (FBS), 2 mM L-glutamine, penicillin (100 IU/ml) and streptomycin (100 μg/ml).

Retrovirus Production and Transduction for Cyno iPS Cell Generation.

Separate retroviral vectors containing coding sequences for four human transcription factors, OCT4 (GenBank Accession NM_{—}002701), SOX2 (GenBank Accession NM_{—}003106), KLF4 (GenBank Accession NM_{—}004235), and c-MYC (GenBank Accession NM_{—}002467), respectively, were produced in PLAT-A packaging cells. Two different backbone plasmids (pMX and pBMN) that are based on Moloney Murine Leukemia Virus (MMLV) were acquired from Cell Biolabs, Inc. and Allele Biotechnology, respectively. In addition, coding sequences for Canis familiaris telomerase reverse transcriptase (dTERT; GenBank Accession AF380351) and simian virus 40 (SV40) Large T-antigen (LT) (GenBank Accession NC_{—}001669) were cloned into commercially available retroviral expression vectors that were made Gateway®-compatible (Invitrogen). These constructs were used in some experiments to generate cyno iPS cells, as described in Example 2. For certain experiments using mouse cells, separate retroviral vectors containing coding sequences for four mouse transcription factors, OCT4 (GenBank Accession AK145321 [NM_{—} 013633]), SOX2 (GenBank Accession NM_{—}011443), KLF4 (GenBank Accession NM_{—}010637), and c-MYC (GenBank Accession NM_{—}001177354), respectively, were produced in PLAT-A packaging cells. Twenty-four hours before transfection, PLAT-A cells were plated at a density of 6×10⁶ cells per 10 cm plate. The cells were transfected with the retroviral vectors with Fugene 6 transfection reagent (Roche). Twenty-four hours before transduction, cyno skin fibroblasts were plated at a density of 4×10⁵ cells per 10-cm plate. Forty-eight hours and seventy-two hours after transfection, the retroviral supernatants were collected, filtered through a 0.45 μM filter and used for double transduction of cyno skin fibroblasts on two consecutive days to enhance transduction efficiency. The fibroblasts were transduced with the viral supernatants supplemented with 4 μg/ml polybrene. Four days after transduction, the fibroblasts were trypsinized and replated at 0.3×10⁵ cells per 10 cm dish on irradiated MEF (CF-1 or B6) feeder layers on top of gelatin-coated plates. The next day, the serum-containing medium was replaced with a cyno iPS cell culture medium (serum-free; i.e., DMEM/F12 containing 20% (v/v) KOSR (KO serum replacement, Invitrogen), 2 mM L-glutamine, 0.1 mM non-essential amino acids (NEAA), 0.1 mM β-mercaptoethanol, and 20 ng/ml bFGF (Invitrogen)). Valproic acid (VPA, 1 mM) was added to media on days 5-11 of reprogramming. Around two to three weeks after transduction, the colonies with ES cell-like morphology were picked and transferred into 24-well, 12-well, and 6-well plates for further expansion and analyses. During passaging, the colonies were dissociated into small clumps of cells either mechanically (using a needle or pipette tip) or enzymatically (collagenase IV, 1 mg/ml in DMEM, Invitrogen, #17104-019).

Real Time PCR (qPCR).

Total RNA was isolated using RNeasy mini kit (Qiagen) and was treated with DNase I (Qiagen) to remove potential genomic DNA contamination. 2 μg of DNAse I-treated total RNA was reverse transcribed using the High Capacity cDNA Reverse Transcription Kit (Applied Biosystems) in a 40 μl volume. The cDNA was diluted to 40 ng/ul with sterile water containing 100 ng/μl glycogen for qPCR analysis. The qPCR reaction was performed in triplicate using 40 ng of cDNA in a 10 μl reaction volume containing 1× Taqman Universal PCR Master Mix (Invitrogen), 500 nM primers, 300 nM probe. Each sample was also normalized against β-actin as an internal control to generate ΔCt. Linear fold change in mRNA expression was determined by ΔΔCt method (Applied Biosystems). The Nanog mRNA expression was analyzed by acquiring the relative quantification [RQ=2̂−(ΔΔCt)]. relative to the calibrator sample, cyno iPS 11 line (Type III). The primer sequences for qPCR reactions were the following (“F”=forward primer; “R”=reverse primer); Nanog F, 5′-GAC AGC CCC GAT TCT TCC A-3′(SEQ ID NO:3); Nanog R, 5′-TCT TCC TTT TTT GTG GCA CTA TTC T-3′(SEQ ID NO:4); Nanog probe (FAM/BHQ), 5′-CCC AAA GGC AAA CAA CCT ACT GCT GCA-3′(SEQ ID NO:5); ACTB F, 5′-ACC CAC ACT GTG CCC ATC TAC-3′(SEQ ID NO:6); ACTB R, 5′-GCT CAG TGA GGA TCT TCA TGA GGT A-3′ (SEQ ID NO:7); ACTB probe (FAM/BHQ), 5′-CTG GCT GGC CGG GAC CTG AC-3′ (SEQ ID NO:8); SV40 LT F, 5′-TGC TCA TCA ACC TGA CTT TGG A-3′ (SEQ ID NO:9); SV40 LT R, 5′-CAC TGC TCC CAT TCA TCA GTT C-3′ (SEQ ID NO:10); SV40 LT probe (FAM/BHQ), 5′-TTC TGG GAT GCA ACT GAG ATT CCA ACC T-3′ (SEQ ID NO:11); Exogenous Oct4 F, 5′-CCC ATG CAT TCA AAC TGA GGT AA-3′ (SEQ ID NO:12); Exogenous Oct4 R, 5′-TGG CCT GCC CGG TTA TTA-3′ (SEQ ID NO:13); Exogenous Oct4 probe (FAM/BHQ), 5′-TCC AGC TGA GCG CCG GTC G-3′ (SEQ ID NO:14).

Lentivirus Production.

For Gluc reporter gene expression, the lentivirus encoding Gaussia princeps luciferase (Gluc) was packaged in 293T-6E cells. Two lentiviral vectors were cloned for constitutive and tetracycline-inducible expression, respectively, of Gluc from human CMV promoter, together with a blasticidin or neomycin resistance gene as a selectable marker, respectively. For ADCC target gene expression, two other lentiviral vectors encoding cyno Her2 or human CD20, respectively, under transcriptional control of an EF-1α promoter, and with a puromycin resistance gene as a selectable marker, were packaged in 293T-6E cells.

Alkaline Phosphate, Immunofluorescence, and Immunohistochemical (IHC) Staining.

Alkaline phosphate (AP) activities were measured using alkaline phosphate staining kit (Stemgent), according to the manufacturer's instruction. For immunofluorescence staining, cells were fixed in 4% (v/v) paraformaldehyde for 20 minutes, washed three times with phosphate buffered saline (PBS), and blocked with PBS containing 10% (v/v) goat or donkey serum and 0.1% (v/v) Triton X-100 for 1-2 hours at room temperature. Cells were then stained with primary antibodies in PBS containing 2% (v/v) FBS at 4° C. overnight, followed by three times-washing in PBS and incubation with secondary antibodies for 2 hours. Formalin-fixed paraffin-embedded graft sections (cyno grafts) were stained with SV40 LT IHC. The following primary antibodies were used: OCT4 (1:100, Stemgent), KLF4 (1:100, Santa Cruz), SOX2 (1:100, Stemgent), c-MYC (1:100, Millipore), SSEA-4-Alexa Fluor 555 (1:100, BD biosciences), TRA-1-60-Alexa Fluor 488 (1:100, Stemgent), Nanog (1:100, Bethyl), βIII-tubulin (1:100, Santa Cruz or 1:500, TUJ1), SMA (1:1000, Sigma), Pan-Cytokeratins (pan-CK, 1:50, C-11, Cell Signaling), CDX2 (1:100, BioGenex), PDX1 (1:50, Abcam), Sox17 (1:100, R&D Systems), FoxA2 (1:100, Millipore), and SV40 LT (BD Pharmingen 554149). Nuclei were counterstained with DAPI.

Immunoblotting.

Cell pellets and graft fragments were lysed in RIPA buffer (Pierce 89901) supplemented with protease inhibitor (Roche). Tumor fragments were lysed using the Tissue Lyser machine (Qiagen) for 3 cycles of 30 seconds. Both tumor and cell lysates were allowed to lyse completely on a Nutator™ mixer (TCS Scientific Corporation) at 4° C. for 30 minutes. Lysates were cleared of cell debris and quantified using a BCA assay (Pierce 23225). 19.2 μg of protein was loaded onto bis-tris gels and transferred onto nitrocellulose membrane for blotting. Blots were blocked for 1 hour, at room temperature with the respective blocking buffers containing varying concentrations of bovine serum albumin (BSA) and skim milk, depending on the antibody being used. Primary antibodies were incubated at 4 degree overnight at their respective concentrations and buffers. Secondary antibodies were incubated for 1 hour at room temperature (RT) in their respective buffers. The following primary antibodies were used: Cytokeratins (pan-CK, C-11, Cell Signaling), Vimentin (Dako M0725), SMA (Sigma A5228), N-Cadherin (BD 610920), E-Cadherin (BD 610181), and beta-Actin (Sigma A1978).

In Vitro Differentiation.

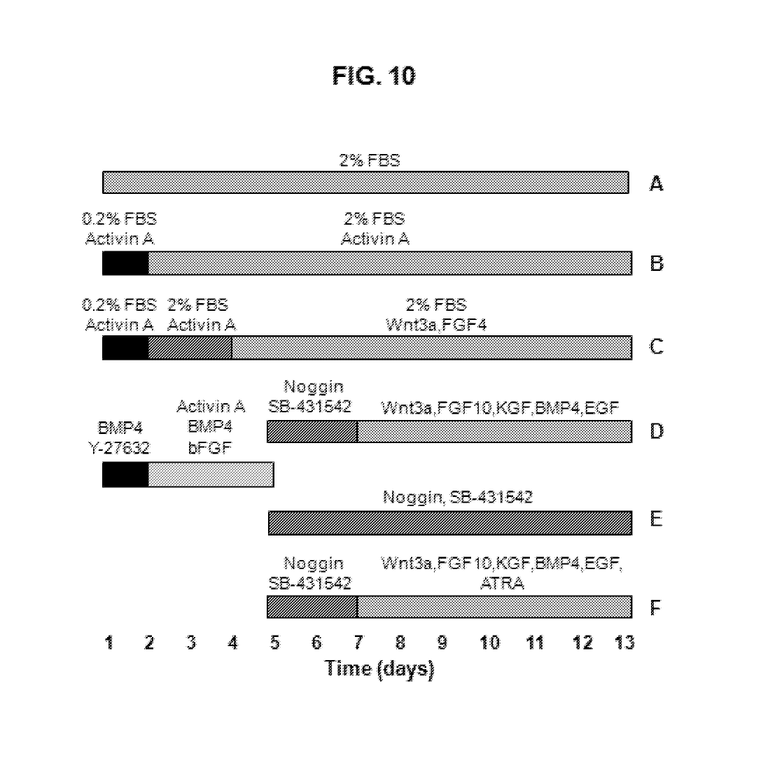

For embryoid body (EB) formation, clumps of cyno iPS cells were plated on low attachment 6-well plates in a cyno iPS cell culture medium without bFGF for 10-14 days. The floating EBs were collected and plated on 0.1% gelatin-coated 24-well plates to differentiate in serum (20% (v/v) FBS)-containing media for another 10-14 days. The resulting differentiated cells derived from EBs were fixed and stained for three germ layer lineages including ectoderm, mesoderm, and endoderm. To generate autologous cyno target cells, either a single cyno iPS line or multiple cyno iPS cell-like lines were differentiated into enriched epithelial-like cells (cyno iPS-EPI cells) through multiple passages under a serum condition (10% (v/v) FBS). Cyno gut-like cell differentiation was performed as described in Example 2 and FIG. 10 herein. For definitive endoderm (DE) differentiation, cyno iPS cells were cultured in RPMI 1640 medium containing 100 ng/ml activin A with increasing concentration of FBS (0%, 0.2%, and 2% (v/v)) for 3 days.

To optimize conditions to differentiate and enrich cyno iPS cell-derived DE cells into foregut- and hindgut-like cells, we cultured cyno iPS cells under six different conditions (methods A-F illustrated in FIG. 10) and compared the gut-specific marker expression. In methods A-C represented in FIG. 10, cyno iPS cell colonies were dissociated into small clumps of cells using needles and were transferred directly onto Matrigel™-coated plates (BD Biosciences), where the cells were treated with various growth factors in DE medium (i.e., RPMI-1640, pH 7.4, supplemented with GlutaMAX™ (Invitrogen), penicillin (100 IU/ml) and streptomycin (100 μg/ml), and 2% (v/v) FBS). In methods D-F illustrated in FIG. 10, cyno EBs derived from cyno iPS cells were collected, dissociated into single cells using dispase, and plated onto Matrigel™-coated plates, where the cells were treated with various growth factors in DE medium. In method A, 2% (v/v) FBS and no growth factor was used. In method B shown in FIG. 10, where the high enrichment of foregut-like cells was derived from cyno iPS cells, the cyno iPS cell clumps were cultured in 100 ng/ml Activin A-containing medium with increasing concentration of FBS (0.2 and 2% (v/v)) at days 1-13. In method C shown in FIG. 10, where the high enrichment of hindgut-like cells was derived from cyno iPS cells, the cyno iPS cell clumps were treated with 100 ng/ml Activin A-containing medium with increasing concentration of FBS (0.2 and 2% (v/v)) at days 1-3, and were then further cultured with Wnt3a (500 ng/ml) and FGF4 (500 ng/ml) at days 4-13. The following concentrations of growth factors were used in methods D-F for certain time periods (shown in FIG. 10): 100 ng/ml Activin A; 10 μM Y-27632; 10 ng/ml bFGF; 0.5, 1, 10 ng/ml BMP4; 200 ng/ml Noggin; 10 μM SB-431542; 100 ng/ml Wnt3a; 10 ng/ml FGF10; 10 ng/ml KGF (FGF-7); 10 ng/ml EGF.

Flow Cytometry Analysis.

The flow cytometry analyses were performed to examine target gene expression in the cyno target cells (cyno iPS-EPI lines). The quantitative analysis of cell surface antigen expression (Her2 and CD20 target genes) was performed by QIFIKIT® (DAKO (K0078))-based flow cytometry following the manufacturer's instructions. FACS analyses were performed on a FACS LSRII using the following labeled primary antibodies: anti-CD20-FITC (BD Biopharmingen, clone 2H7, BD 555621), anti-Her2-PE (BD; Becton, Dickinson and Company, clone 9G6, BD 554300), anti-CD45-PE (BD), anti-CD34-APC (BD), and mouse IgG2b(κ)-FITC isotype control (BD Biopharmingen). The parental lines without CD20 transduction were used as negative controls for CD20 immunostaining. The unstained lines were used for negative controls for Her2 and CD20 immunostaining.

Cyno NK Sensitivity (Antibody Independent Cellular Cytotoxicity (AICC)) and Antibody-Dependent Cellular Cytotoxicity (ADCC) Assays.

Cynomolgus peripheral blood mononuclear cells (PBMC) were obtained from SNBL (Everett, Wash.). A total of 24 ml of whole blood was drawn into sodium heparin tubes for each donor animal, and PBMCs were isolated from whole blood. NK cells were isolated from the PMBCs by positive selection, using CD159a antibody and the EasySep isolation kit (StemCell Easy Sep PE selection kit, cat #18551). The NK cells from each donor were counted and resuspended at 2×10⁶ cells/mL in complete DMEM for use in the AICC and ADCC assays. Viable target cells (10⁷) were labeled with a concentration of CFSE (Invitrogen cell tracking kit, V 12883) optimized for each cell type and resuspended at 0.4×10⁶ cells/ml in complete DMEM for use in the AICC and ADCC assays. The AICC and ADCC assays were performed in a 96 well round bottom tissue culture plate (Corning 3799). CFSE-labeled target cells (T) were added, 50 μL to contain 20,000 cells. Cyno NK cell effectors (E) were added, 50 μL to contain 100,000 cells (5:1 E:T). Cultures were incubated for 18 hours at 37° C. followed by assessment of target cell cytotoxicity assayed using flow Cytometry. CFSE⁺,7AAD⁺ target cells represent those cells that are killed. For the 100% lysis controls, the complete content of several wells that contain targets+effectors only were harvested, washed once in an ice cold 80% methanol, and resuspended in 7AAD (7-Amino Actinomycin D) solution, and the number of dead target cells was assessed by flow cytometry. For the ADCC assay, antibodies were titrated from 1 μg/mL to 0.00001 μg/mL by carrying 10 μL in 100 μL of complete DMEM containing 10% FCS (a 1:10 dilution).

Statistical Analyses for Cyno AICC and ADCC.

For the AICC analysis, percent (%) specific lysis was defined as (T+E lysis %−T alone lysis %)/(100% T lysis−T alone lysis %)×100. For the ADCC analysis, percent (%) specific lysis was defined as (experimental lysis %)−(spontaneous lysis %)/(100% lysis−spontaneous lysis %)×100. Spontaneous lysis was determined by wells containing only targets+effectors (no antibodies). The 100% lysis was determined by wells where targets+effectors had been lysed by washing once with ice cold 80% (v/v) methanol. Experimental lysis values came from wells the contained the test antibody and targets+effectors.

Gaussia princeps Luciferase (Gluc) Assay.

To determine the sensitivity of the Gluc assay in quantitative assessment of iPS-derived target cells, conditioned media from different numbers of cyno iPS-derived cells expressing Gluc were assayed with coelenterazine (Prolume), Gluc substrate, for Gluc activities at different cell numbers after 24 hours of culture or at various time points. For Gluc activity assay, 50 μl of conditioned culture medium was transferred into 96 white or black opaque wells. Immediately after adding 50 μl of 20-μM coelenterazine into conditioned media, Gluc activities were measured for 10 seconds of integration time using a plate luminometer (Envision).

Animal Care and Welfare.

Gender, strain, species, age and/or weight were care for in accordance to the Guide for the Care and Use of Laboratory Animals, 8^{th} Edition. Animals were group housed at an AAALAC, Intl-accredited facility in (STERILE/NON-STERILE) ventilated micro-isolator (or static) housing on corn cob bedding. All research protocols were approved by the appropriate Institutional Animal Care and Use Committee (IACUC). Animals had ad libitum access to pelleted feed and water via automatic watering system or water bottle. Animals were maintained on a 12:12 (or other)-hour light: dark cycle in rooms and had access to enrichment opportunities. All animals were determined to be specific pathogen-free for mouse parvovirus, Helicobacter, etc.

Cell Injection and Graft Formation in Mice.

To evaluate the growth ability of cells in vivo, the cyno or mouse iPS-EPI cells and their derivative cells (10⁷ cells per mouse, n=5) were subcutaneously injected into NSG (NOD scid gamma, NOD.Cg-Prkdc^{scid} Il2rg^{tm1Wjl}/SzJ, 005557, JAX), nude, or B6 (C57BL/6, Harlan) mice depending on the cell being tested (female, 6-12 weeks old). Cells were trypsinized with 0.05% trypsin and neutralized with DMEM medium containing 10% heat inactivated fetal bovine serum (FBS). Cells were pelleted and washed 1× with cold unsupplemented DMEM medium. Cells were resuspended to a final concentration of 10⁷ cells in 100 ul of a 1:1 mixture of DMEM and BD Matrigel™ (BD 354234.) Cell suspension was injected subcutaneously using a 1-ml syringe and a 27 gauge needle into the upper left ventral area of NSG or B6 mice depending on the cell type being tested. Graft or tumor measurements were taken by caliper 1-2×/week depending on rate of graft (tumor) growth. Graft/tumor volume was measured by calipers using the equations: volume=x*y*z and volume=4/3*π*(x/2)*(y/2)*(z/2). The x, y, and z represent the length, width, and height of graft.

Animal Care and Welfare at Charles River (Reno, Nev.).

Animals (Chinese cynomolgus macaques) were housed in stainless-steel cages. Primary enclosures were as specified in the USDA Animal Welfare Act (9 CFR, Parts 1, 2 and 3) and as described in the Guide for the Care and Use of Laboratory Animals. The targeted conditions for animal room environment were as follows: Temperatures (64° F. to 84° F.), humidity (30% to 70%), Ventilation (Greater than 10 air changes per hour, with 100% fresh air (no air recirculation)), and 12-hour light/12-hour dark photoperiod. Purina Certified Primate Diet No. 5048 was provided daily in amounts appropriate for the size and age of the animals. This diet was be supplemented with fruit or vegetables at least 2 to 3 times weekly. No contaminants were known to be present in the certified diet at levels that would interfere with the results of this study. All animals used on study had documentation to confirm at least one negative serum antibody test to simian retrovirus (SRV). In addition, all samples were further tested for SRV by PCR analysis. All of the studies complied with all applicable sections of the Final Rules of the Animal Welfare Act regulations (Code of Federal Regulations, Title 9), the Public Health Service Policy on Humane Care and Use of Laboratory Animals from the Office of Laboratory Animal Welfare, and the Guide for the Care and Use of Laboratory Animals from the National Research Council. The protocol and any amendments or procedures involving the care or use of animals in all of the studies were reviewed and approved by the Testing Facility Institutional Animal Care and Use Committee (Charles River, Reno, Nev.) before the initiation of such procedures. The Testing Facility's attending veterinarian was responsible for implementation of programs for the evaluation of the health status of study animals, the recommendation of treatment for health conditions, the evaluation of response to treatment, as well as the diagnosis of pain or distress.

Cyno Cell Injection, Graft Implantation, Graft Measurement, and Graft Removal in Cyno Monkeys.

The autologous iPS cell-derived target cells (e.g. cyno iPS-EPI cells and their derivatives) were subcutaneously injected into the autologous cyno monkeys located in Charles River Laboratories (Reno, Nev.). An appropriately sized needle (˜22 gauge) connected to a syringe was used for cell injection. The autologous target cells were injected into the scapular or lumbar regions of the back from the mid-dorsal line to the flank. The volume for each dose (2-5 ml for 1×10⁷ to 3×10⁷ cells) was administered in a single injection within the demarcated area. For the autologous cyno graft implantation to cyno monkeys, NSG mice were previously injected with the cyno iPS-derived cell lines. The resulting cyno grafts were harvested and implanted into the subcutaneous space of the autologous cyno monkeys. Graft measurements were taken 1×/7-10 days and graft volume was measured by the caliper and ultrasound using the equation: volume=4/3*π(x/2)*(y/2)*(z/2). The x, y, and z represent the length, width, and height of graft. At the end of each study, the grafts were removed by either 6-mm skin biopsy punch or an elliptical incision around a graft to collect the whole graft including connective tissues for histology and molecular analyses. The skin incision or biopsy site was closed by using appropriately sized monofilament absorbable suture in a subcuticular pattern. A topical antibiotic ointment was applied to surgical site post-surgery. Following graft removal, the animals received an initial dose of Hydromorphone (0.1 mg/kg, intramuscular [IM]) prior to surgery and a second dose approximately 4-6 hours later. In addition, buprenorphine (0.03 mg/kg, IM) was administered approximately every 8-12 hours beginning the evening of graft removal and continuing for 2 days.

### Example 2

### Generation of Autologous Non-Human Mammalian Models and Method of Monitoring Exogenously Introduced Cells

In order to generate an autologous non-human mammalian model, for example, in a non-human primate, we first generated cyno iPS cells by reprogramming cyno somatic cells, such as skin fibroblasts which can be easily obtainable from live animals. These differentiated adult somatic cells could be reprogrammed into a pluripotent state by ectopic expression of four human transcription factors, OCT4, SOX2, KLF4, and c-MYC (FIG. 2A-B).

Generation of Cyno iPS Cells.

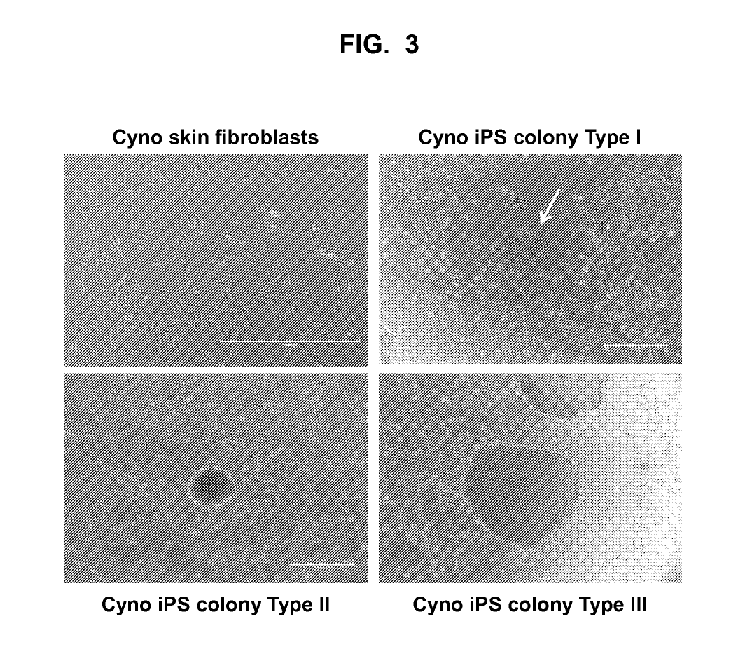

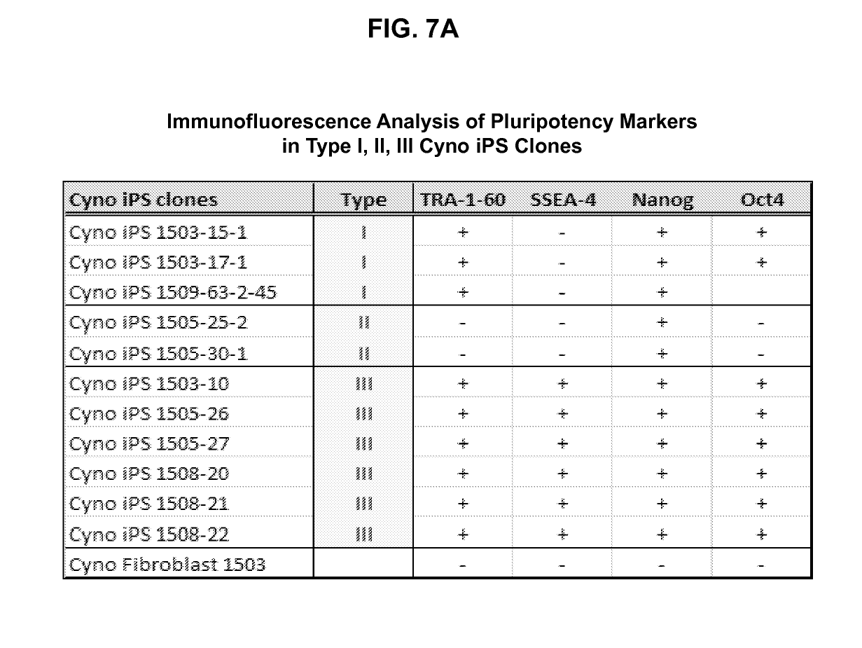

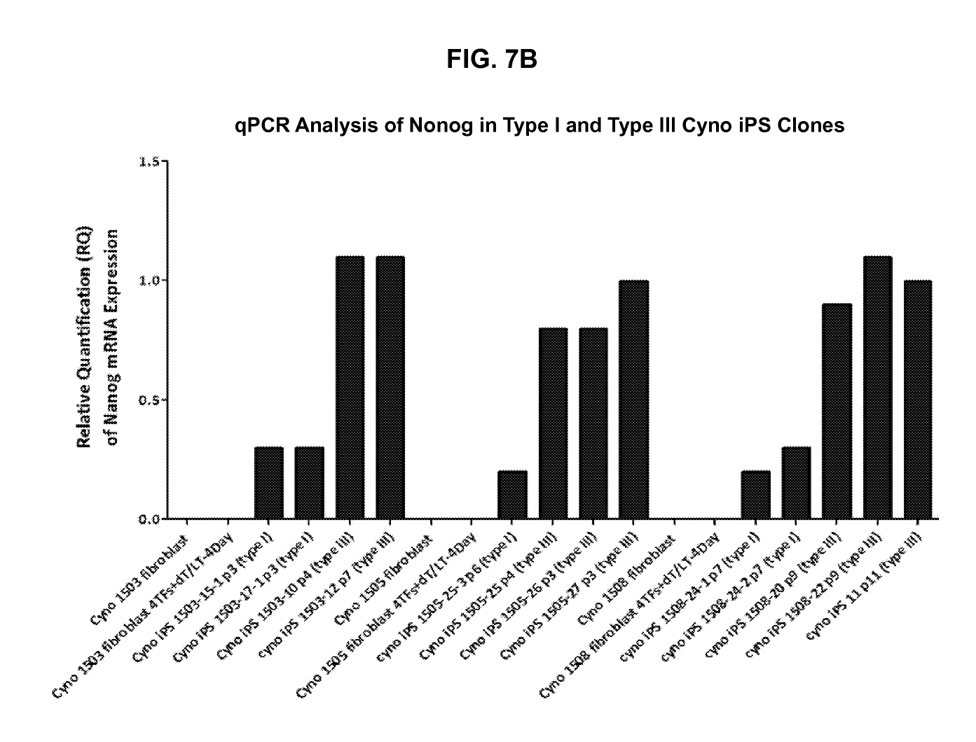

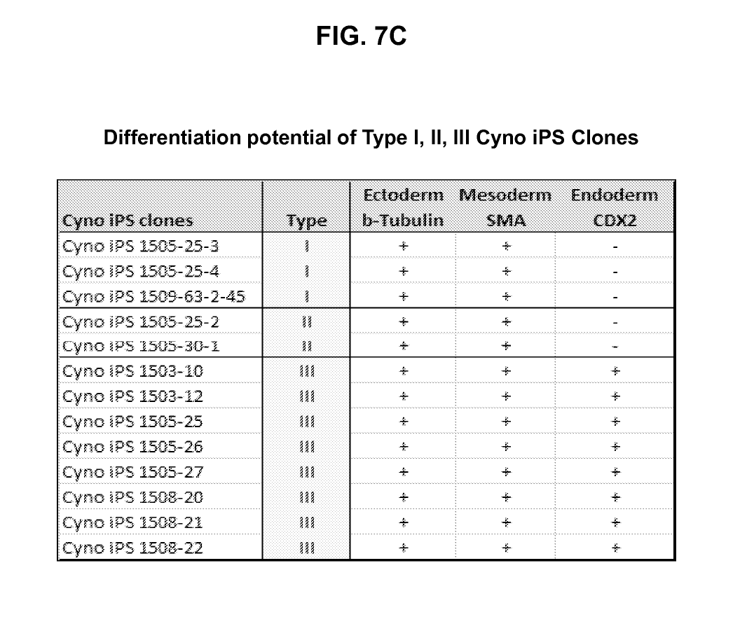

The cyno fibroblasts were isolated and expanded from dorsal skins of female cyno monkeys (FIG. 3A). We examined the transduction efficiency of the retrovirus carrying these four factors in cyno skin fibroblasts. Retroviruses from two different backbone plasmids (pMX and pBMN) that are based on Moloney Murine Leukemia Virus (MMLV) were produced in PLAT-A packaging cells and yielded 20-50% transduction efficiencies in cyno fibroblasts (FIG. 2A-B). As some human iPS cell studies showed that human telomerase reverse transcriptase (hTERT) and SV40 LT may enhance the reprogramming efficiencies by affecting indirectly supportive cells (e.g., Park, I. H. et al., Reprogramming of human somatic cells to pluripotency with defined factors, Nature 451, 141-146 (2008)), cyno iPS cell generation was generated by using six factors, i.e., the four human factors OCT4, SOX2, KLF4, and c-MYC and the catalytic subunit of dog telomerase reverse transcriptase (dTERT) and SV40 large T antigen (SV40 LT). Four days after transduction, the cells were replated onto irradiated mouse embryonic fibroblasts (MEF) feeder cells at 0.3×10⁵ cells per 100 mm dish. This cell density resulted in a good spacing between the colonies with which the reprogrammed colonies could be selected efficiently. The next day, the serum-containing medium was replaced with a cyno iPS cell culture medium supplemented with basic fibroblast growth factor (bFGF). The transduced fibroblasts underwent the drastic changes in morphology. Around day 14 to 21 after transduction, the colonies appeared morphologically similar to human ES/iPS cell and cyno ES cell colonies. Among these cyno iPS cell-like colonies, we observed three distinctive morphological types of colonies (type I, type II, and type III), all of which formed tightly packed and flat colonies that resembled human ES/iPS cell and cyno ES cell colonies under phase contrast microscopy (FIG. 3). Type I colonies had packed cells with visible individual cells under phase contrast microscope. Type II colonies also contained densely packed cells, but formed domed colonies, and occasionally had dark brown cells in the middle of colonies when viewed under phase contrast microscope. Type III colonies also contained densely packed cells with no visible individual cells but had bright tight colony borders with no dark centers. In order to distinguish between fully reprogrammed cyno iPS cells and partially reprogrammed cells, we further examined these different types of colonies and validated them through pluripotent marker expression and differentiation potential (see sections for FIG. 7A-C).

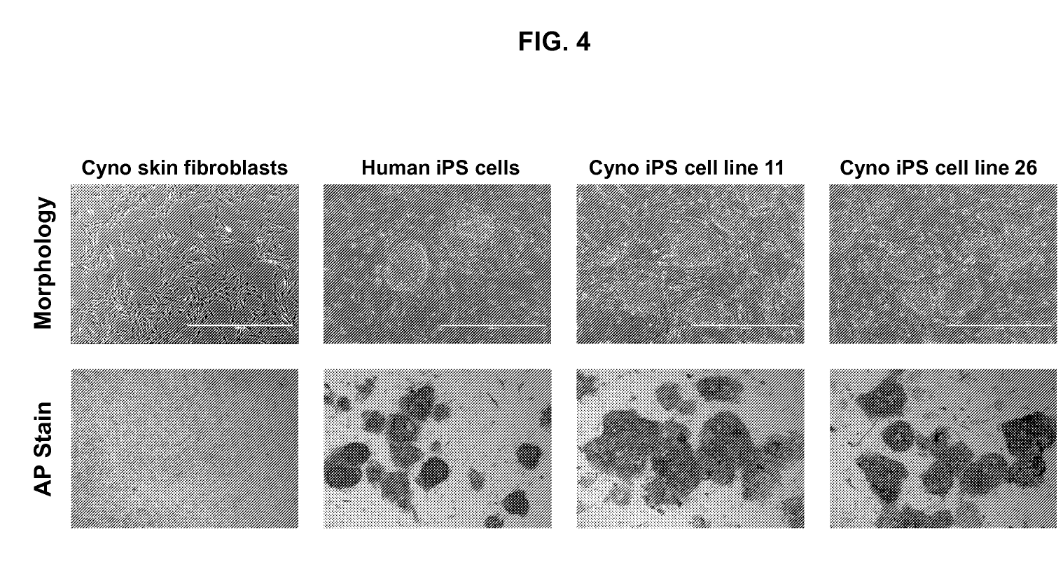

Undifferentiated pluripotent stem cells, such as ES and iPS cells, express high levels of alkaline phosphatase (AP) that decreases upon differentiation. In the early passages, we observed the heterogeneous populations in the iPS cell cultures, which were evidenced by mixed populations of AP+ and AP− colonies. Thus, to isolate bona fide cyno iPS cells, we further selected iPS colonies based on ES cell-like morphology and remove the spontaneously differentiated colonies in serial passages. Most subclones of cyno iPS cell lines in later passages (after 6-7 passage) showed homogeneous populations with ES cell-like morphology (FIG. 4) and AP+ colonies (FIG. 4), as shown in the positive control, human iPS cells. The parental cyno skin fibroblasts failed to express the pluripotency marker AP (FIG. 4).

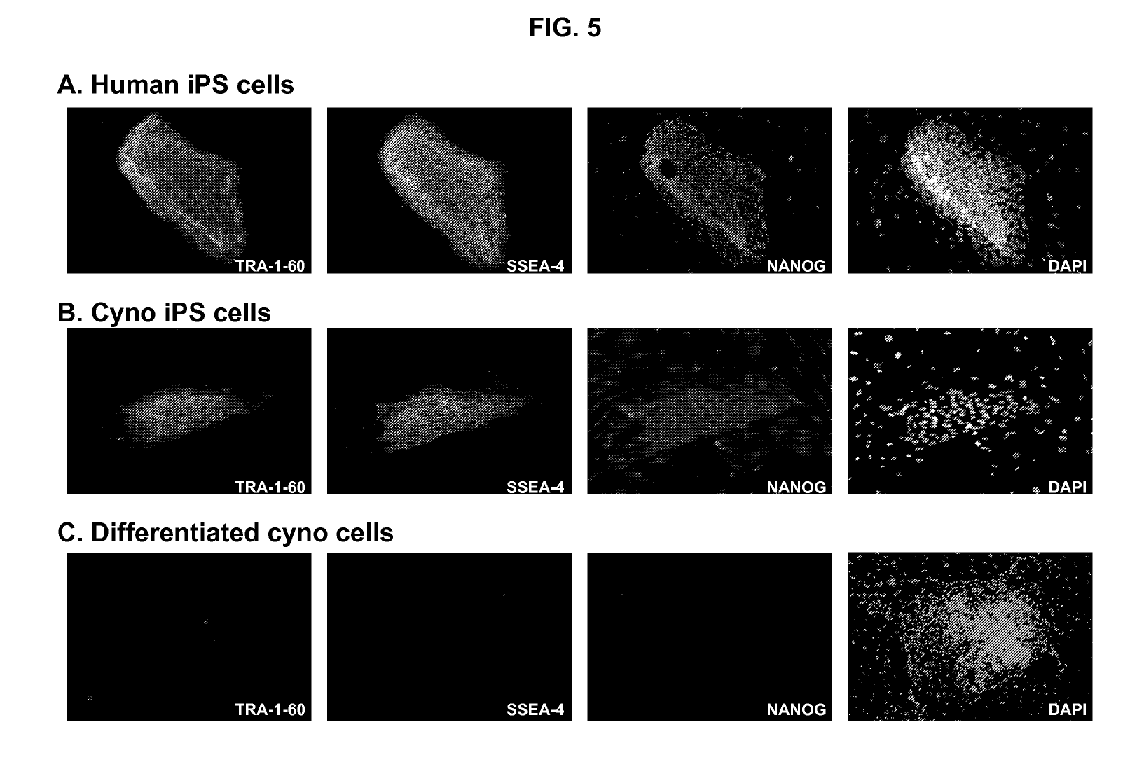

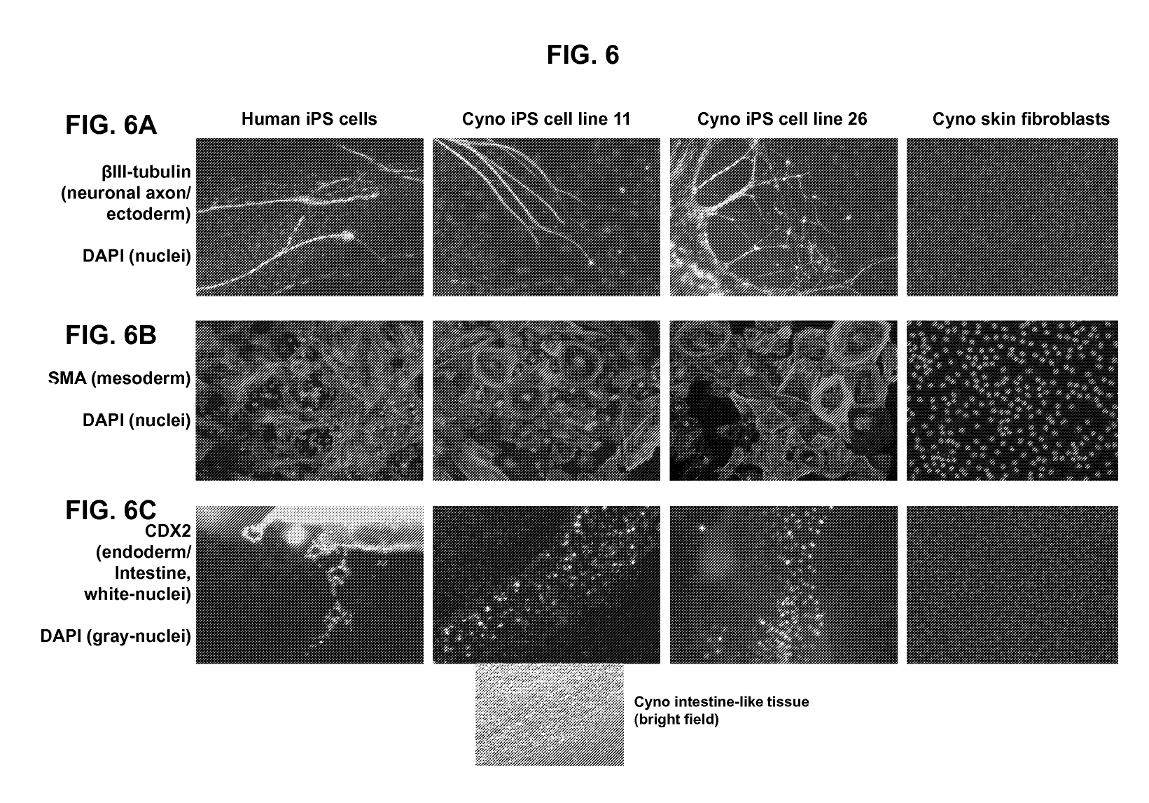

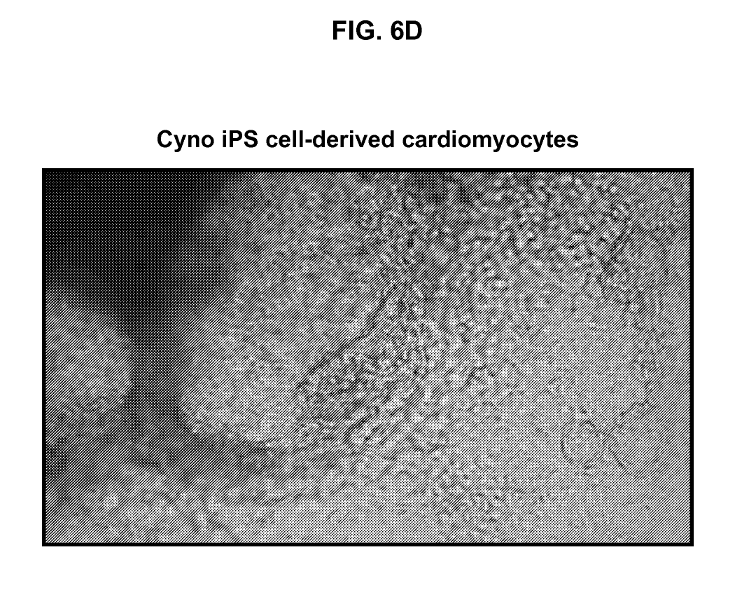

In addition to AP staining, we demonstrated the pluripotency of cyno iPS cells by staining them with other pluripotency markers. The fully reprogrammed cyno iPS cell lines (cyno iPS 11, reprogrammed from SNBL cyno fibroblasts; FIG. 5B) expressed TRA-1-60, TRA-1-81, SSEA-4, and NANOG pluripotency markers which are highly expressed in human ES/iPS and cyno ES cells, whereas none of these genes were expressed in differentiated cyno colonies (FIG. 5C). We also validated the pluripotency of cyno iPS cells by examining differentiation potential of iPS-derived embryoid bodies (EBs) into all three germ layer lineages, including ectoderm, mesoderm, and endoderm, a key property of pluripotent stem cells, like ES cells. We generated EBs from cyno iPS cells under floating conditions for 10-12 days and then transferred them into gelatin-coated plates to grow in serum-containing media for another 10-14 days. Around day 4 after plating EBs into gelatin-coated plates, neuronal axons (ectoderm), or neuron-like cells, were differentiated from cyno iPS cells, which were evidenced by immunofluorescence staining for βIII-tubulin expression (FIG. 6A). Mesodermal cells were differentiated from cyno iPS cells, as indicated by immunostaining for α-Smooth Muscle Actin (SMA) (FIG. 6B). Cyno iPS cells also exhibited differentiation potentials into endodermal cells, which were evidenced by CDX2 expression (FIG. 6C). Notably, the immmunostaining for CDX2, specific for hindgut lineages, revealed that intestinal tissues with canal-like, or column-like, structures were differentiated from cyno iPS cells (FIG. 6C), indicating hindgut-like cells. The parental cyno skin fibroblasts failed to display a differential potential to any of lineages (FIGS. 6A-C). In addition, the cyno iPS cell lines were able to differentiate into cardiomyocytes (beating heart cells; FIG. 6D), which demonstrates the differential potential of these cyno iPS cells into multiple cell types (FIG. 6D). By establishing reprogrammed, pluripotent cyno iPS cell lines, we can make these iPS cells differentiate into any type of autologous target cells of interest. After we established cyno iPS generation methods, we started to generate autologous cyno iPS cells. We isolated fibroblasts from cyno skin biopsies acquired from cyno monkeys in Charles River which were designated for our studies. Upon reprogramming of fibroblasts, we obtained different morphological types of cyno iPS colonies (Type I, II, and III) as described above. To distinguish between fully reprogrammed iPS cells and partially reprogrammed iPS cells, pluripotent marker expression and differentiation potential were examined for these colonies. Immunofluorescence analysis of pluripotency markers showed that type I cyno iPS colonies (clones) were TRA-1-60⁺ SSEA-4⁻ Nanog⁺ Oct4⁺, and type II cyno iPS clones were TRA-1-60⁻ SSEA-4⁻ Nanog⁺ Oct4⁻, and type III cyno iPS clones were TRA-1-60⁺ SSEA-4⁺ Nanog⁺ Oct4⁺ (FIG. 7A). The cyno fibroblasts (prior to the reprogramming) did not express any of these pluripotent markers as expected. As Nanog was expressed in all of three types of iPS clones, we examined the level of Nanog mRNA expression in different types of cyno iPS clones. Real-Time PCR (qPCR) analysis displayed that the type III cyno iPS clones express 2.7-5.5 fold higher expression of Nanog than type I cyno iPS clones (FIG. 7B). Next, to determine the differentiation potential of these different types of cyno iPS clones, we examined whether these cyno iPS clones can differentiate into all three germ layer lineages, including ectoderm, mesoderm, and endoderm. EB-derived differentiation assays showed that the type III cyno iPS clones possess the differential potential into all three germ layer lineages, whereas type I and type II cyno iPS clones were able to differentiate into ectoderm and mesoderm, but not endoderm (FIG. 7C). Taken together, these results revealed that the type III iPS colonies were the fully reprogrammed iPS colonies, whereas type I and type II colonies were partially reprogrammed iPS colonies. Both fully and partially reprogrammed iPS lines were used to generate target cells in this study.

Generation of Cyno iPS-Derived Target Cells.

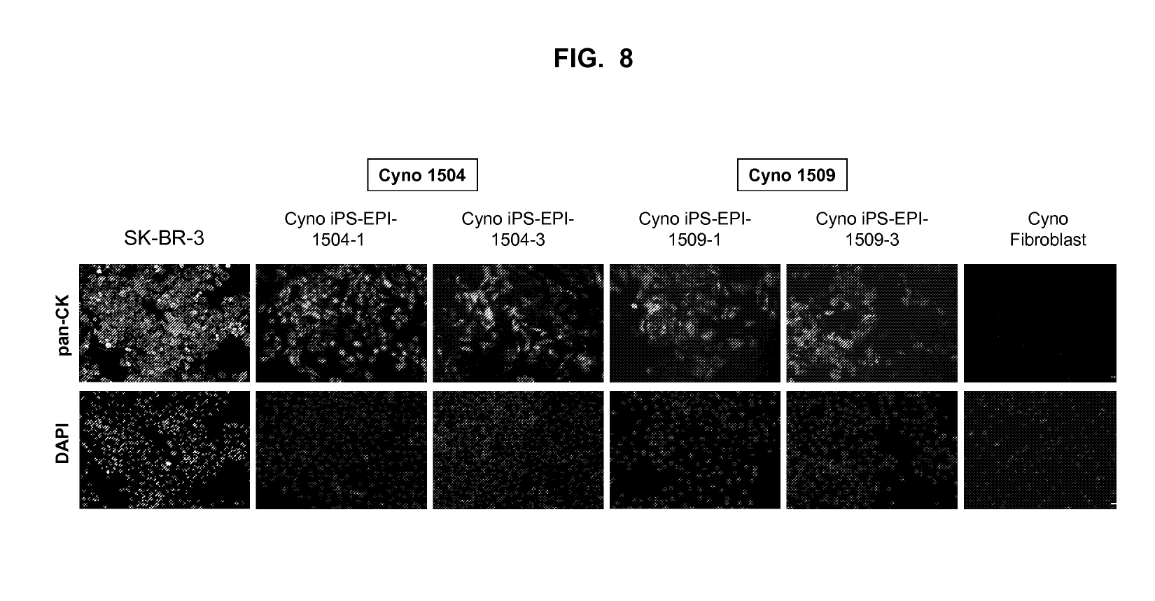

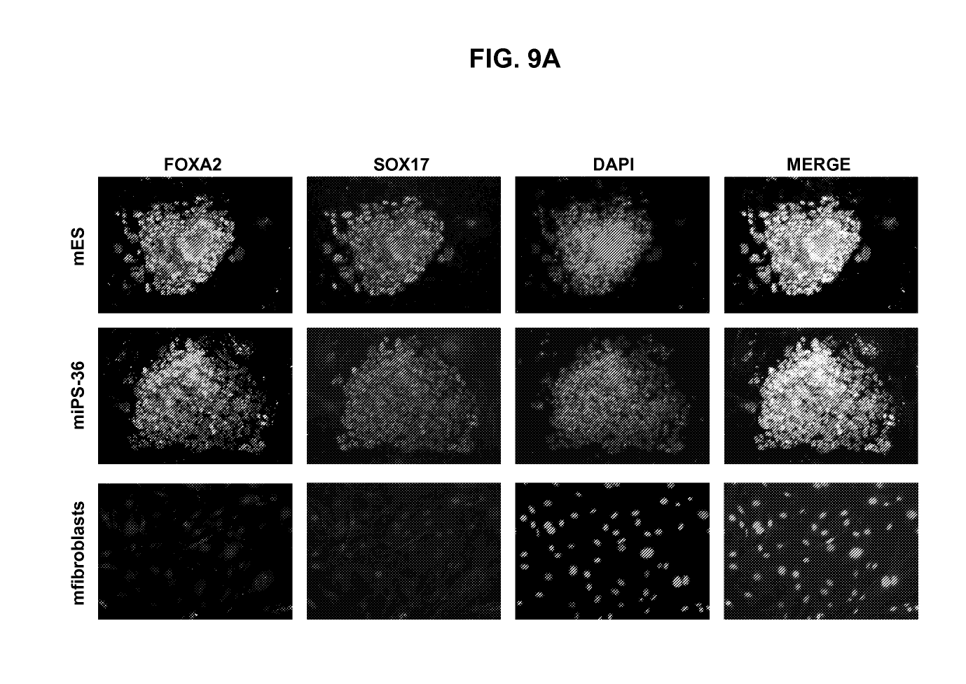

The autologous cyno iPS cells reprogrammed from skin fibroblasts were further differentiated into autologous cyno target cells. For the generation of cyno iPS-derived target cells, we took two different strategies. The first strategy employed to generate autologous cyno target cells was the differentiation of cyno iPS cells into heterogeneous and enriched epithelial-like cells (termed as cyno iPS-EPI cells) through multiple passages under serum-containing (10% (v/v) FBS) culture medium conditions, as described in Example 1 herein. As the majority of carcinomas originate from epithelial cells, these cell types can be useful target cell types of interest for the development of predictive disease models of cancer. We generated cyno iPS-EPI cells from two cyno monkeys using two different methods. One method used a single cyno iPS cell line, and the other method used multiple (more than two) cyno iPS cell-like lines to differentiate into epithelial-like cells (cyno iPS-EPI-1 and cyno iPS-EPI-3, respectively) (FIG. 8). Both methods resulted in generation of highly proliferative epithelial-like cells in cell morphologies, which showed fast growth rates with a short (˜25-32 hours) doubling time in vitro under the serum-containing growth condition. We also examined the expression of epithelial-specific marker, pan-cytokeratin (pan-CK), in cyno iPS-EPI cells. Method 1 appeared to generate more homogeneous epithelial cells (FIG. 8). Method 2 appeared to generate more heterogenous epithelial cell types. SK-BR-3, a luminal breast cancer cell line was used as a positive control for high expression of pan-CK. Cyno fibroblasts, original cells prior to the reprogramming was used as a negative control cell line for pan-CK.

The second strategy employed for autologous cyno target cell generation was the differentiation of cyno iPS cells into specific cell types such as gut-like cells with more homogeneous populations under specific growth factor conditions, so that the differentiated cells can be used in specific disease models of interest (see FIGS. 10, 11 and 12). We have been particularly focusing on generating gut-like epithelial-like cells, because these types of cells can be cellular progenitors of tumor types of interest and, thus, are useful for therapeutic development. These gut-like cells include foregut (anterior part of GI tract that gives rise to esophagus, trachea, lung, stomach, liver, biliary system, and pancreas, etc.) and, midgut (mid-part of GI tract giving rise to the small intestine) and hindgut (posterior part of GI tract that gives rise to the large intestine, including colon, cecum, and rectum, etc).

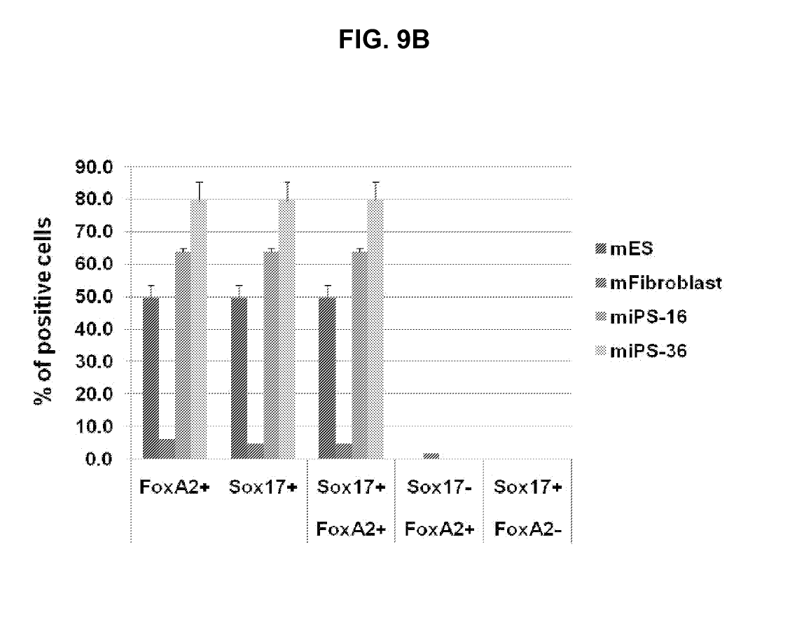

First, we differentiated mouse, cyno and human iPS cells into definitive endoderm (DE) that is a precursor endoderm for organ tissues, and we further differentiated the definitive endoderm into gut-like cells including foregut- and hindgut-like cells using the protocols for days 1-4 of methods B or C, respectively (as illustrated in FIG. 10). Treatment of mouse iPS cells with a high concentration of activin A (a nodal-related TGF-β molecule) and an increasing concentration of serum for 3 days led to differentiation of iPS cells into definitive endoderm and resulted in high enrichment (˜80%) of the cells co-expressing the definitive endoderm markers, SOX17 and FOXA2 (FIG. 9A-B; see, Spence, J. R. et al., Directed differentiation of human pluripotent stem cells into intestinal tissue in vitro, Nature 470:105-109 (2011)).

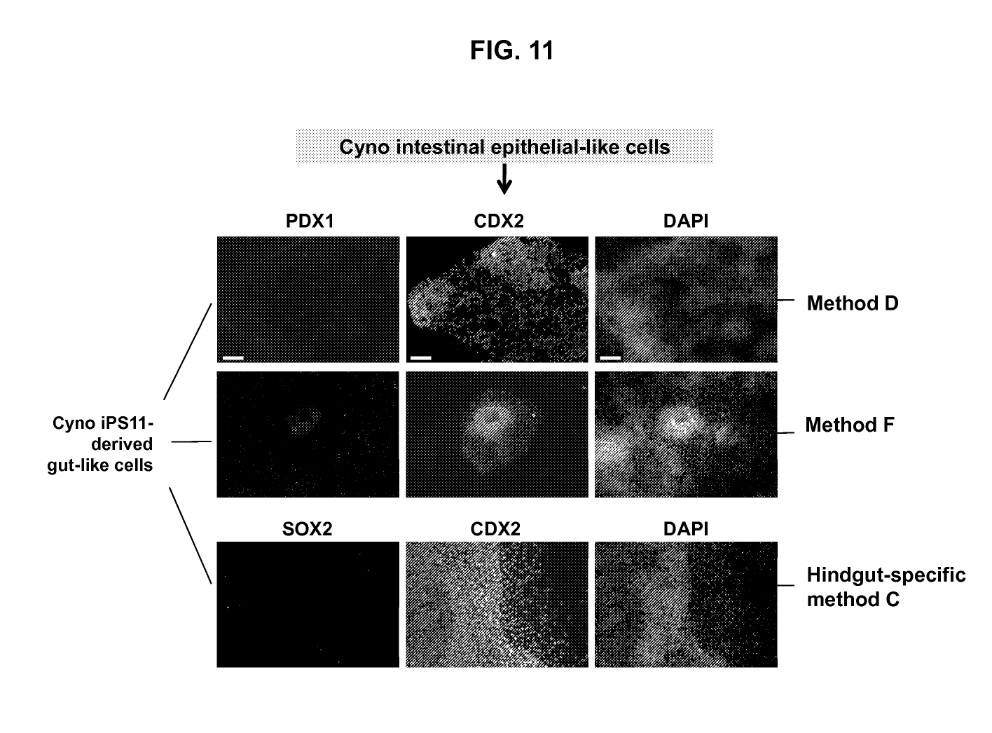

The definitive endoderm can continue to differentiate into specific organ lineages including foregut, midgut, and hindgut. Comparative analysis of several differentiation methods (FIG. 10) revealed that the treatment of a 3-day-activin A-induced DE derived from cyno iPS cells with posteriorizing factors, such as Wnt3a and FGF4 (method C in FIG. 10), promoted differentiation into cyno hindgut-like cells, demonstrating high enrichment (˜98%) of hindgut-like cells (CDX2+ intestinal epithelial-like cells) and almost no foregut-like cells (˜0% of SOX2+ epithelial-like cells) (FIG. 11). Although Wnt3a and FGF4 were previously used in differentiation of human ES and iPS cells into the intestinal tissue (Spence, J. R. et al., Directed differentiation of human pluripotent stem cells into intestinal tissue in vitro, Nature 470:105-109 (2011)), they had not previously been assessed for cyno hindgut specification by differentiation of cyno iPS cells. These CDX2⁺ cyno cells appeared to build intestinal lining-like organoids, which are typically seen in the epithelial lining of intestinal tissues. Although methods D and F in FIG. 10 did not result in high enrichment of either CDX2⁺ hindgut-like cells or SOX2⁺ foregut-like cells, some of cyno epithelium derived from cyno iPS cells under these conditions matured into intestine-like epithelium containing columnar structures (FIG. 11).

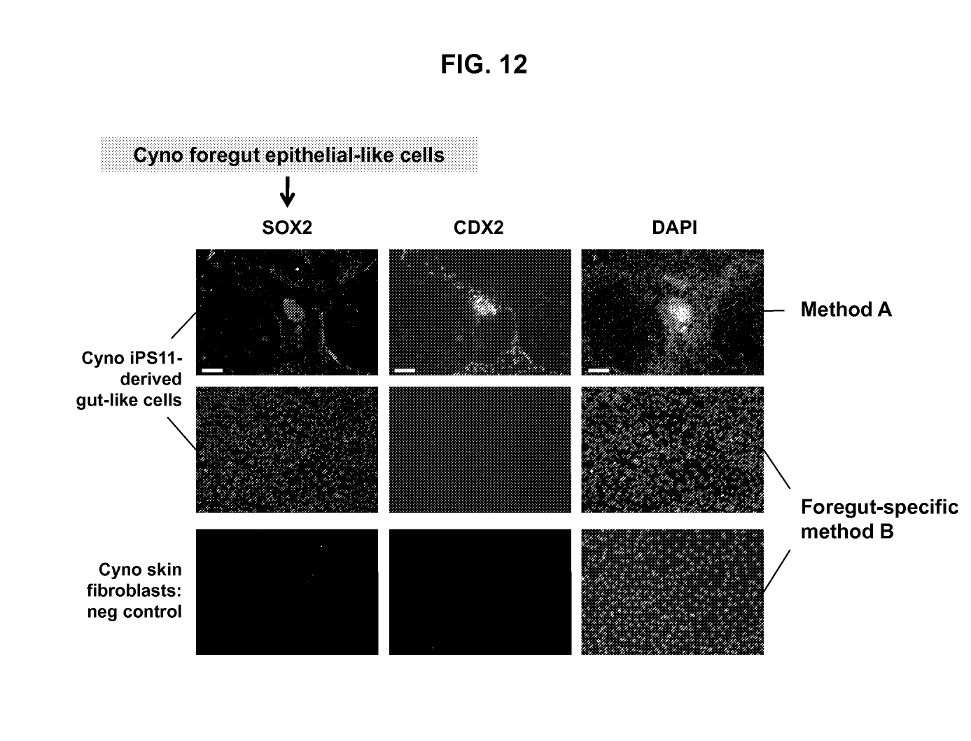

Another analysis of differentiation methods showed that the continuous treatment of cyno iPS cells with a high concentration of activin A after a 3-day-activin A-induced DE formation (method B in FIG. 10) led to high enrichment (˜93%) of cyno foregut-like cells (SOX2⁺ or PDX1⁺ epithelial-like cells) and generated almost no hindgut-like cells (˜0% of CDX2⁺ cells); this indicated cyno foregut specification of cyno iPS cell differentiation under the conditions of method B (see, FIG. 12). Interestingly, the growth factors and compounds used in methods D and F (FIGS. 10 and 11) that were previously tested for differentiation of human ES and iPS cells into anterior foregut endoderm (Green et al.) did not lead to high enrichment of cyno foregut endoderm upon differentiation of cyno iPS cells. The parental cyno skin fibroblasts failed to differentiate into any of gut-specific cells, evidenced by a lack of expression of the gut-specific markers under any differentiation conditions tested (method A-F in FIG. 10), confirming no differential potential of the fibroblast cells (FIG. 12).

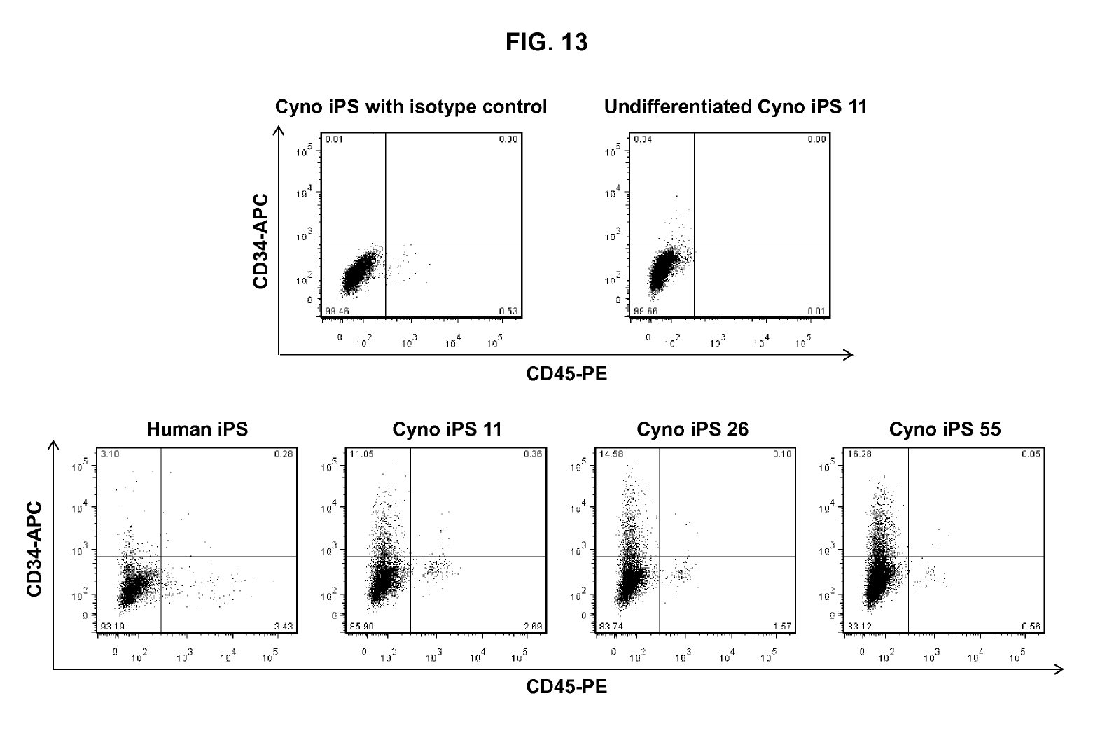

In order to generate hematopoietic cells derived from iPS cells, we first demonstrated the ability of cyno iPS cells to differentiate into CD34⁺ hematopoietic progenitor-like cells (HPCs) which can further give rise to most of blood cell types (hematopoietic lineages). To induce differentiation, cyno iPS cells were co-cultured with mouse bone marrow-derived stromal cells (M2-10B4, ATCC), as used in hematopoietic differentiation of human ES cells and human iPS cells (Ni Z et al., Human pluripotent stem cells produce natural killer cells that mediate anti-HIV-1 activity by utilizing diverse cellular mechanisms. J Virol. 85:43-50 (2011)). At day 14 of co-culture, flow cytometry analysis showed that no cyno CD34⁺ or CD45⁺ cells were generated. At day 32 of co-culture, flow cytometry analysis revealed that 11-16% cyno CD34⁺ hematopoietic progenitor-like cells and 0.6-3% of CD45⁺ leucocytes (white blood cells) were differentiated from three cyno iPS cell lines tested (cyno iPS cell lines 11, 26, and 55) (FIG. 13). However, as expected, undifferentiated cyno iPS cells used as a negative control contained a very low frequency (˜0.3%) of CD34⁺ cells and (˜0.01%) CD45⁺ leucocytes (FIG. 13). Interestingly, co-culture of a human iPS cell line with M2-10B4 did not lead to efficient generation of CD34⁺ HPCs (˜3.4%) and CD45⁺ leucocytes (˜3.7%) from human iPS cells (FIG. 13), whereas the previous report showed that a relatively high enrichment (˜24%) of CD34⁺ HPCs was derived from human iPS cells (Ni Z et al., Human pluripotent stem cells produce natural killer cells that mediate anti-HIV-1 activity by utilizing diverse cellular mechanisms. J Virol. 85:43-50 (2011)). The cyno CD34⁺-HPC-like cells can be further differentiated into hematopoietic lineages including NK cells, T cells, or B cells by co-culture with AFT024 (mouse fetal liver-derived stromal line. ATCC), OP9-DL19 (mouse bone marrow-derived stromal line transduced with retroviral Delta-like-1) or OP-9 (mouse bone marrow-derived stromal line, ATCC) and MS-5 (mouse stromal cells, DSMZ), respectively.

Monitoring Introduced Cells In Vivo.

Toward the development of autologous animal models using iPS-derived target cells, we evaluated methods to monitor and quantify the autologous cyno target cells in vivo. We used a secreted Gaussia princeps luciferase (Gluc) as a reporter to monitor the target cells injected into animals in vivo, because Gluc may provide several advantages for cyno in vivo studies. As the Gluc can be secreted from target cells into the blood, the Gluc is readily detectable in blood samples, thus overcoming optical imaging challenges in cyno due to the monkey's thick skin. Gluc has a short in vivo half life (˜20 min; Wurdinger et al., A secreted luciferase for ex vivo monitoring of in vivo processes, Nat Methods 5:171-173 (2008)), resulting in rapid clearance and little accumulation of Gluc over time, which increases accuracy of estimation of the number of live cells at the time of the test. The cyno iPS-EPI cells-expressing Gluc- and/or TetR were generated by transduction with Gluc- and/or TetR-expressing lentivirus. The cyno iPS-EPI_Gluc cells were further engineered by transduction with Her2- or CD20-lentivirus as an ADCC target gene for anti-Her2 huIgG1 and anti-CD20 huIgG1 antibodies, respectively. These transduced autologous target cells can be transplanted back into the original donor cyno monkeys to examine efficacies of therapeutic antibodies for their ADCC activities in this autologous setting.

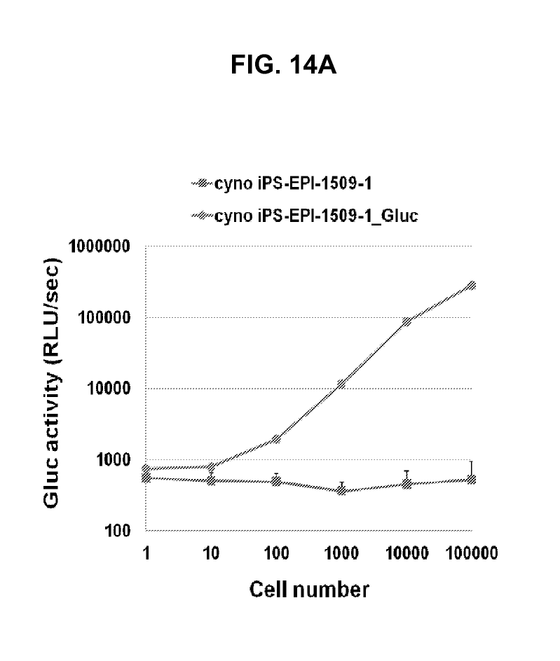

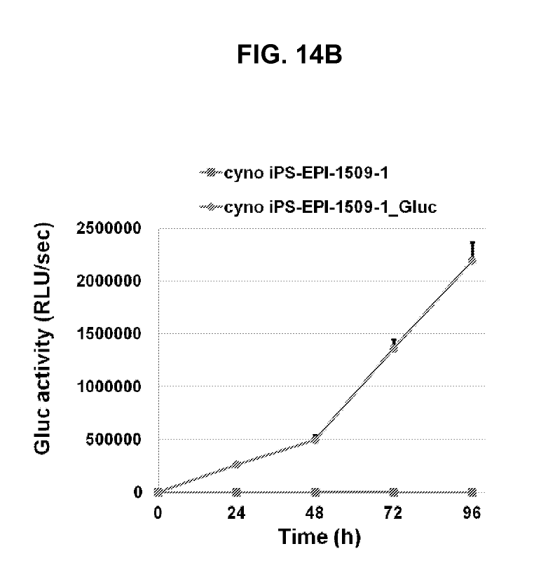

For the further development of tracking methods, we used the autologous cyno iPS-derived epithelial-like cells (cyno iPS-EPI cells) as target cells and we measured activities of secreted Gluc from the target cells. The cyno iPS-EPI cells were transduced with Gluc-lentivirus for constitutive Gluc expression or tet-inducible Gluc expression. To determine the sensitivity of the Gluc assay in quantitative assessment of iPS-derived target cells, conditioned media cultured with different numbers of cyno iPS-derived cells expressing Gluc were assayed using Gluc substrate coelenterazine, to determine Gluc activities after 24 h of culture (FIG. 14A-B). The Gluc assay with a constitutively active cyno iPS-EPI-1509-1_Gluc cell line from cyno monkey 1509 showed an increased, linear range of Gluc activities from diluted cell numbers, whereas parental cyno iPS-EPI-1509-1 line (without Gluc expression) displayed no Gluc activities regardless of cell numbers (FIG. 14A). Significant signals of Gluc activity were detected from ˜1000 transduced cyno iPS-EPI cells, indicating high sensitivity of this Gluc-based tracking method for quantitative estimation of iPS-derived target cells. Next, we tested Gluc activities from 20,000 transduced target cells at different time points. Constitutively active cyno iPS-EPI-1509-1_Gluc line showed an increase of Gluc activity at different time points, whereas no Gluc activity from the parental cyno iPS-EPI-1509-1 line was detected (FIG. 14B).

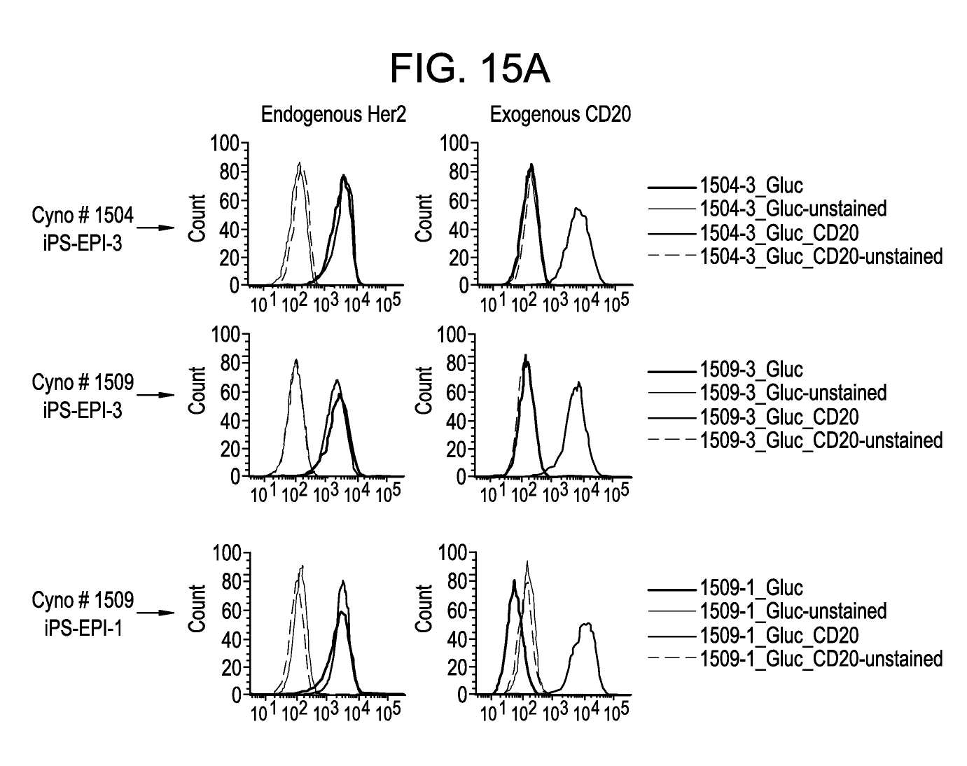

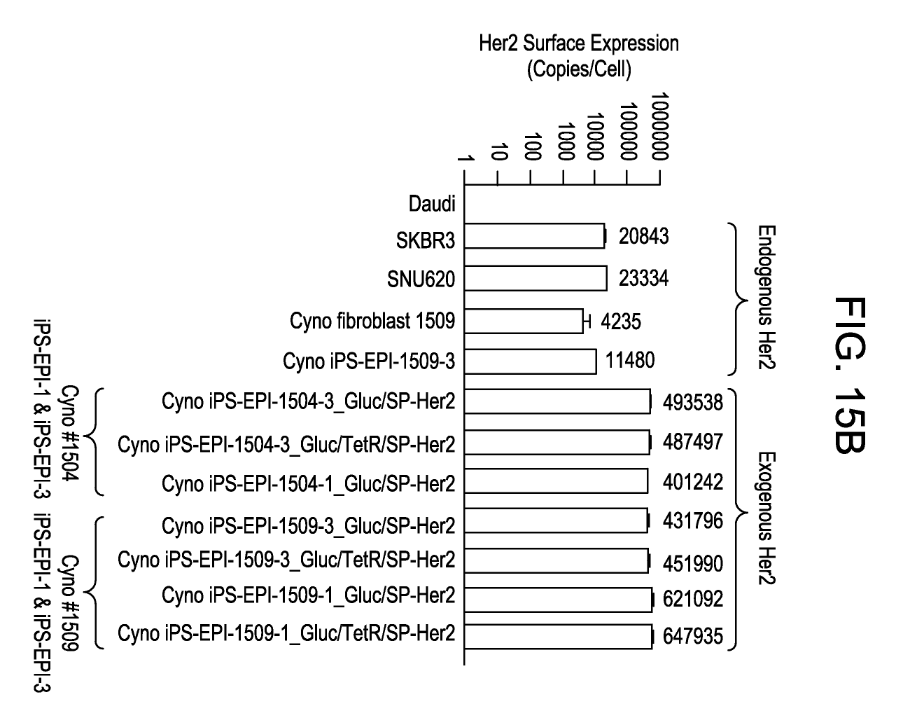

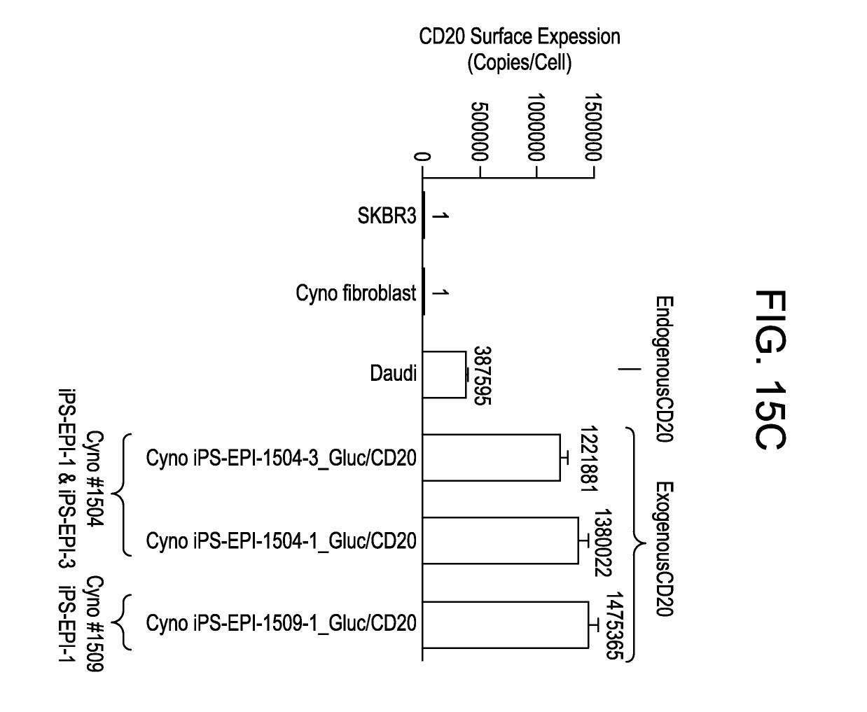

As we found different degrees of heterogeneity from various autologous cyno iPS-EPI lines as described above, we examined the correlation among the degree of heterogeneity, different monkeys, and target gene expression. We transduced the cyno iPS-EPI target cell lines with an ADCC target gene such as CD20. The parental cyno iPS-EPI lines express endogenous Her2. The flow cytometry analysis for examination of the target gene expression in the cyno target cells revealed that the ADCC target genes including exogenous CD20 and endogenous Her2 were expressed at similar levels by different cyno monkeys (1504 and 1509) and various cyno iPS-EPI cell lines (cyno iPS-EPI-1 and cyno iPS-EPI-3 in both monkeys) (FIG. 15A). This result indicates that this cyno model has low variability in the level of target gene expression which can directly affect ADCC activities, supporting the utility of this autologous cyno iPS-derived model for the development of therapeutics. Furthermore, in order to obtain the quantitative analysis of the cell surface antigen expression (Her2 and CD20 target genes), we performed QIFIKIT®-based flow cytometry (FIGS. 15B and 15C). High cell surface expression (˜4×10⁵-6×10⁵ copies/cell) of exogenous Her2 was detected in all of the various cyno iPS-EPI cells transduced with Her2-carrying lentivirus (cyno iPS-EPI-SP-Her2) from both cynos 1504 and 1509 (FIG. 15B). In addition, high cell surface expression (˜1.2×10⁶-1.5×10⁶ copies/cell) of exogenous CD20 was detected in cyno iPS-EPI cells transduced with CD20-carrying lentivirus (cyno iPS-EPI-CD20) from both cynos 1504 and 1509 (FIG. 15C).

In order to select the target cells with better survival and growth in cyno monkeys in vivo, we screened multiple cyno iPS-EPI cell lines based on some key characteristics such as NK sensitivity, antibody-dependent cellular cytotoxicity (ADCC), and growth ability in immunodeficient mice.

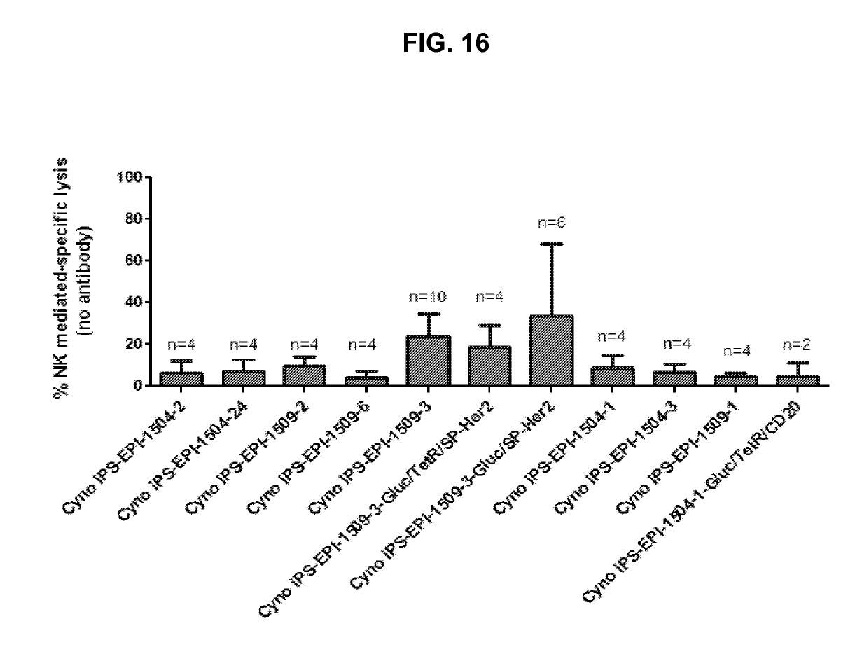

The NK sensitivity was assessed by incubation of various cyno target cell lines (iPS-EPI lines and their derivatives) with cyno NK cells in the absence of antibody. Therefore, the NK sensitivity can be also called antibody independent cellular cytotoxicity (AICC). The cyno NK cells were enriched from cyno peripheral blood mononuclear cells (PBMC) using CD159a antibody. Despite the donor variability in NK effector cells, most of target cells showed a low level of NK-mediated AICC (lower than 10%) (FIG. 16). Cyno iPS-EPI-1509-3 and its derivatives transduced with Gluc, TetR and/or Her2 showed ˜18.8-33.8% (average) of NK-mediated AICC (FIG. 16).

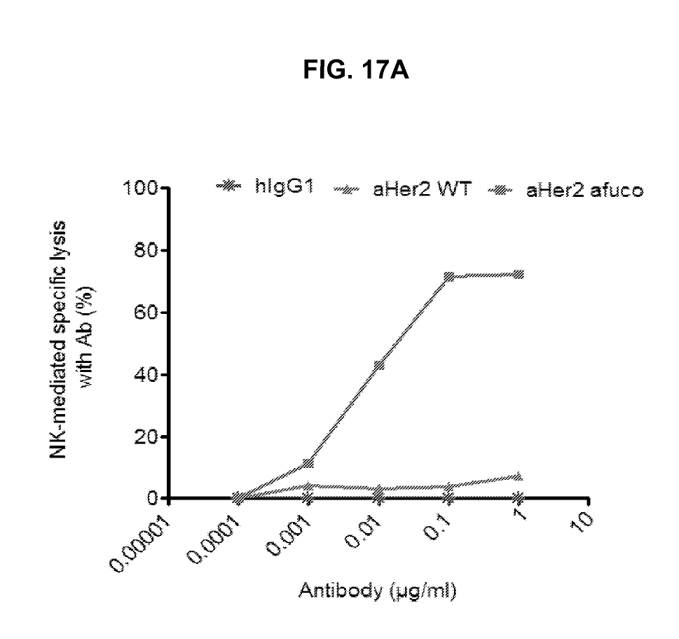

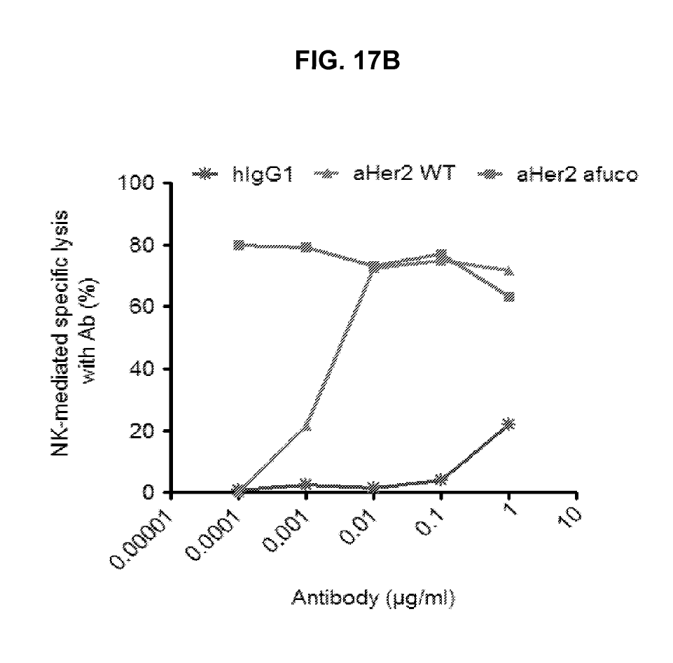

Next, we examined whether these cyno iPS-EPI cells and derivatives can be used as target cells in immune cell-mediated killing assays in the presence of antibody. The ability of anti-Her2 huIgGI antibodies to induce cyno NK-mediated antibody-dependent cellular cytotoxicity (ADCC) against target cells was assessed (FIG. 17A-B). An afucosylated antibody (anti Her2-Afuco) with increased affinity to human FcγRIIIa led to enhanced cyno NK-mediated ADCC activity against target cells-expressing cell surface antigen, Her2. As the cyno iPS-EPI-1509-3 line expresses a moderated level of endogenous Her2 (FIG. 15A-B), only anti-Her2 Afuco was able to induce the potent NK cell-mediated ADCC against cyno iPS-EPI targets, whereas anti-Her2 WT and negative control huIgG1 failed to do so (FIG. 17A). However, when the cyno iPS-EPI-1509-3 line was further engineered to express an exogenous Her2 by lentiviral transduction at the high cell surface expression level (FIG. 15B), both anti-Her2 WT and anti-Her2 Afuco were able to induce NK-mediated ADCC against the target cells (the cyno iPS-EPI-1509-3-Gluc/TetR/SP-Her2) (FIG. 17B). At lower antibody concentrations (0.0001-0.01 μg/ml), anti-Her2 Afuco resulted in enhanced NK-mediated ADCC, compared to anti-Her2 WT (FIG. 17B).

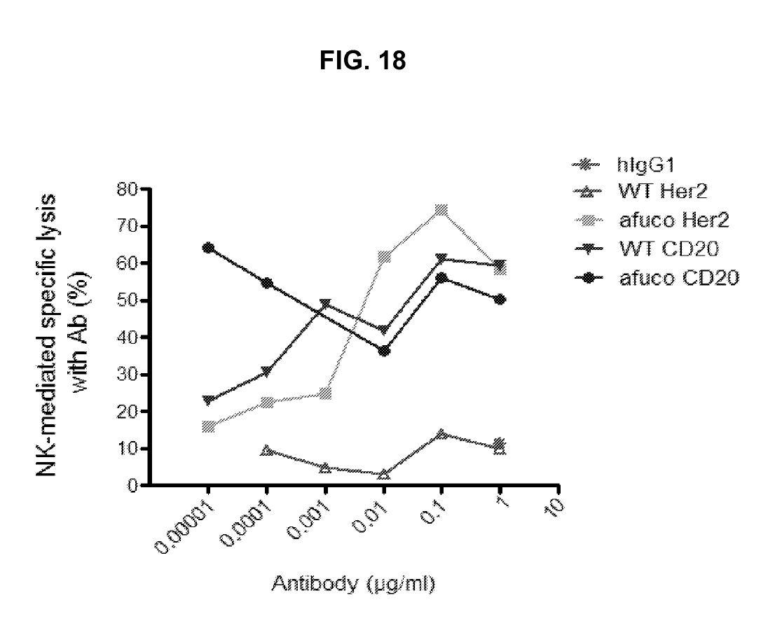

In addition, the ability of anti-CD20 huIgGI antibodies to induce cyno NK-mediated ADCC against target cells was evaluated (FIG. 18). The cyno iPS-EPI-1509-1-Gluc/CD20 used as a target cell line (FIG. 18) express a high level of exogenous CD20 (FIG. 15C) as well as a moderate level of endogenous Her2 (FIG. 15A). As consistent with ADCC results with Her2 endogenously expressing-target cell line (FIG. 17A), anti-Her2 Afuco was able to mediate potent cyno NK-mediated ADCC against the cyno iPS-EPI-1509-1-Gluc/CD20 target cells due to the moderate level of Her2 expression (FIG. 18). In addition, an anti-CD20 Afuco resulted in increased cyno NK mediated-ADCC activities against the target cells-expressing exogenous CD20 at the lower levels of antibody concentration, compared to anti-CD20 WT (FIG. 18). These data demonstrate that the cyno iPS-EPI lines and their derivative cell lines can be used as effective target cells for efficacy studies such as ADCC.

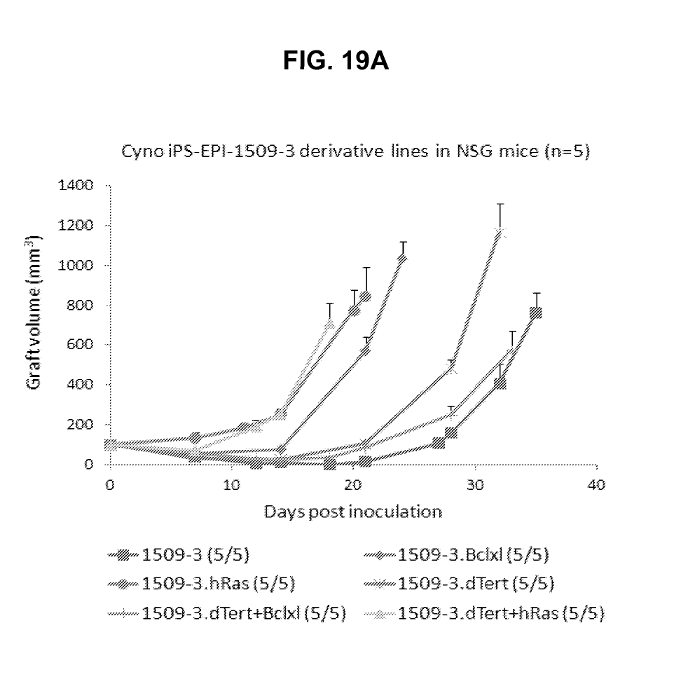

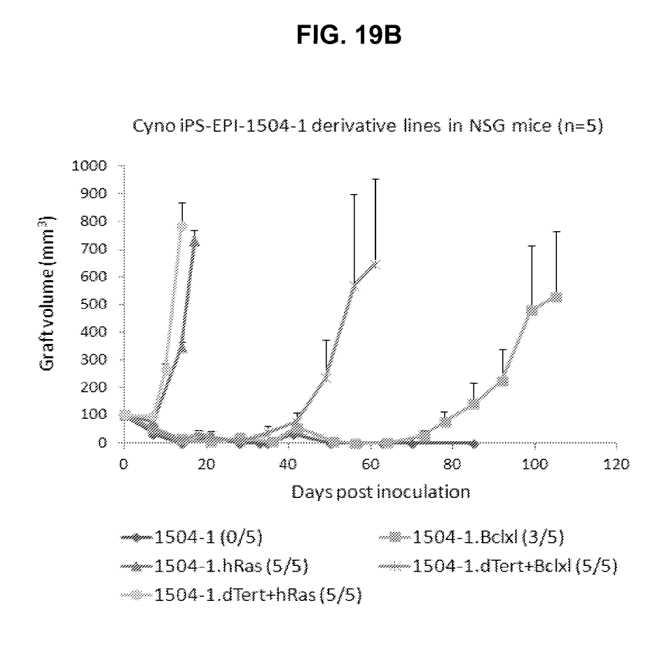

Next, to select the target cells with better survival and growth in cyno monkeys in vivo, we screened multiple cyno iPS-EPI cell lines based on the growth ability in immunodeficient NSG (NOD scid gamma) mice. To this aim, we transformed the target cell lines by transducing them with one or more oncogenes (e.g. HRas and/or SV40 large T antigen) and/or TERT (telomerase reverse transcriptase catalytic subunit), and/or anti-apoptotic genes (e.g. Bcl-xL). Those genes can be introduced into the target cells by either retroviral or lentiviral transduction. Using the resulting transformed cells, we examined whether they can enhance proliferation and/or promote tumorigenicity, and provide more efficient growth potential in vivo, which may enable efficient survival and growth of target cells in immunocompetent animals as well as immunodeficient animals in a desired time frame of preclinical study. We performed either single or double transduction of iPS-EPI cells from cyno 1504 (FIG. 19B) and cyno 1509 (FIG. 19A) by retrovirus carrying HRas, Bcl-xL, or dogTert to generate diverse transformed cell lines. All of the tested target cells also expressed SV40 LT that was introduced during reprogramming into iPS cells. In both 1504 cyno iPS-EPI derivatives and 1509 cyno iPS-EPI derivatives, HRas transduction was able to enhance the growth rates in NSG mice most effectively, while Bcl-xL or dogTert also improve the survival and growth rates of iPS-EPI lines at varying degrees (FIG. 19A and FIG. 19B).

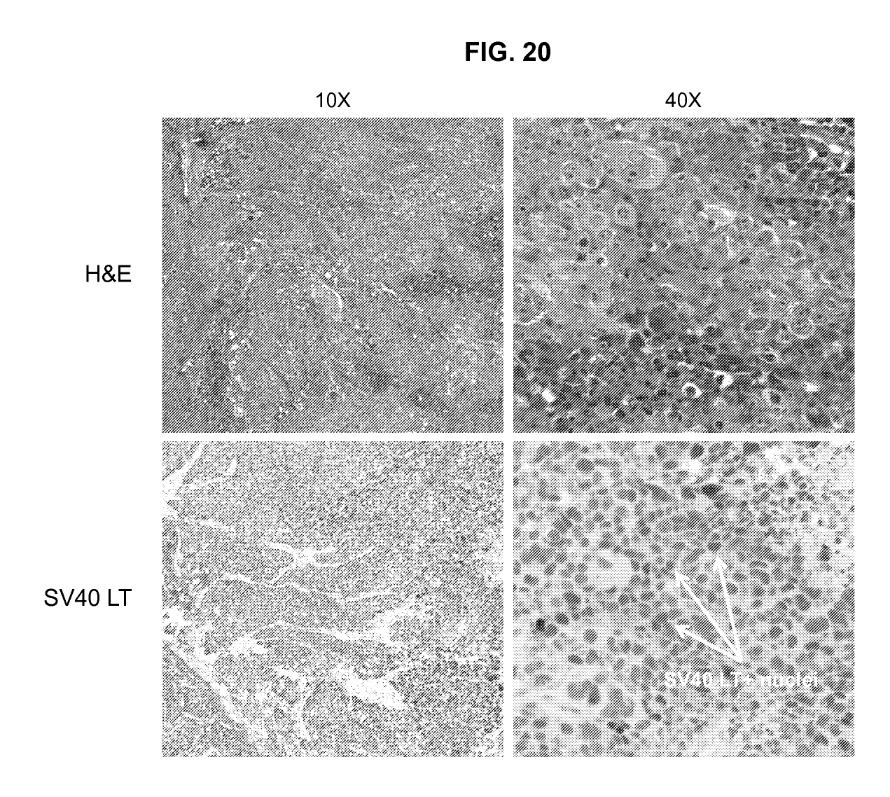

To monitor and confirm the presence of exogenously introduced cyno iPS-EPI cells in the grafts grown in NSG mice, we performed immunohistochemical (IHC) staining for SV40 LT antigen using formalin-fixed paraffin embedded (FFPE) cyno grafts. SV40 LT was used to improve the reprogramming efficiency during the reprogramming process. For the cyno iPS-EPI cells expressing the SV40 LT, this gene can be used as a biomarker to monitor the viable target cells implanted into animals. As expected, the viable cyno cells that were in the majority of the cyno iPS-EPI-1509-3.dTert+Bclxl graft grown in NSG mice showed the high expression of SV40 LT whereas non-viable cells did not express it (FIG. 20).

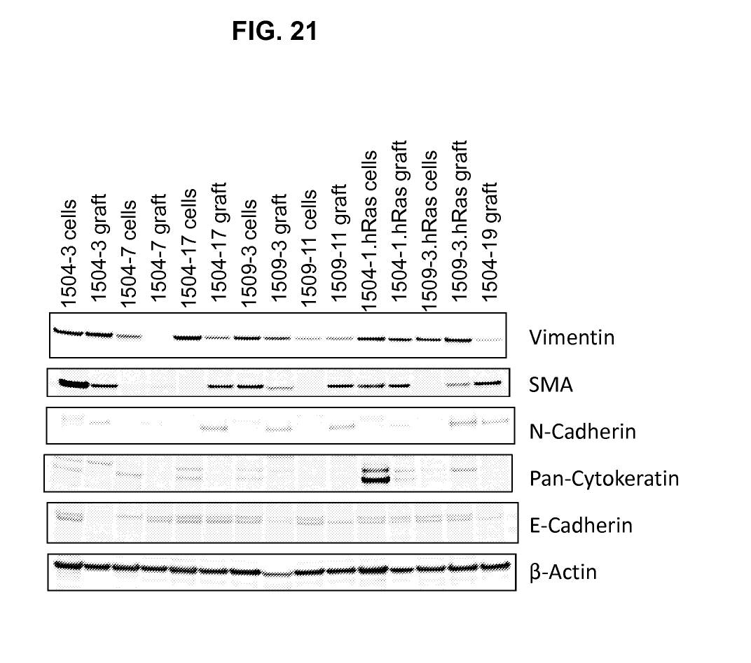

Next, we investigated whether the cyno grafts derived from cyno iPS-EPI cells in NSG mice contained some of cell populations that were potentially enriched and selected during the cell growth and survival in vivo. Indeed, the Western blot analysis revealed the various cyno iPS-EPI grafts grown in NSG mice were more enriched for mesenchymal-like cells (expressing N-cadherin) compared to original cell lines, while the cyno iPS-EPI grafts contained E-cadherin expressing cells similar to the original cells (FIG. 21). Cytokeratins and E-cadherin were used as epithelial cell markers, whereas N-cadherin was used as a mesenchymal cell marker. Vimentin and SMA were used as both epithelial and mesenchymal cell markers.

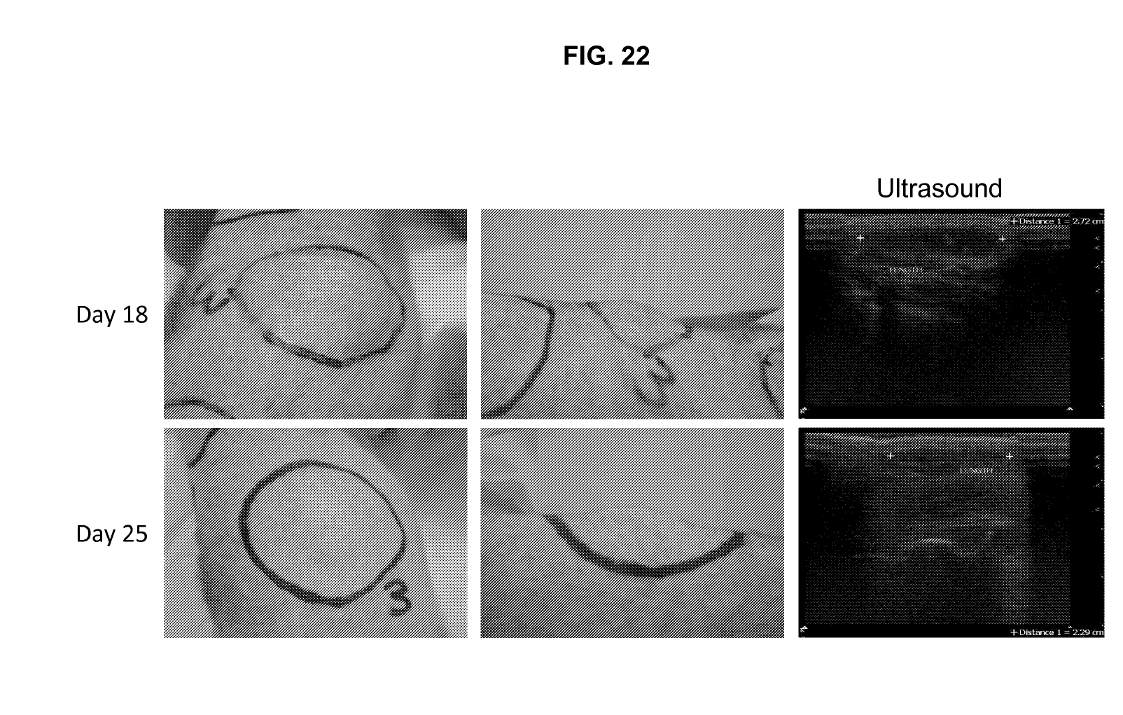

To examine the growth of cells injected into the autologous cynos in vivo, the cyno iPS-EPI cell lines, that were selected based on cyno NK sensitivity, in vitro ADCC activity, and growth rates in NSG mice, were re-injected subcutaneously to the back of original donor cyno monkeys. For example, cyno iPS-EPI-1509-3.HRas cell line was re-injected into the donor cyno monkey 1509 (FIG. 22). Calipers and ultrasound were used to measure the sizes of grafts. The similar sizes of graft (˜2.4 cm³, ˜2 cm³, ˜2 cm³) were measured with calipers at day 18, day 25, and day 31.

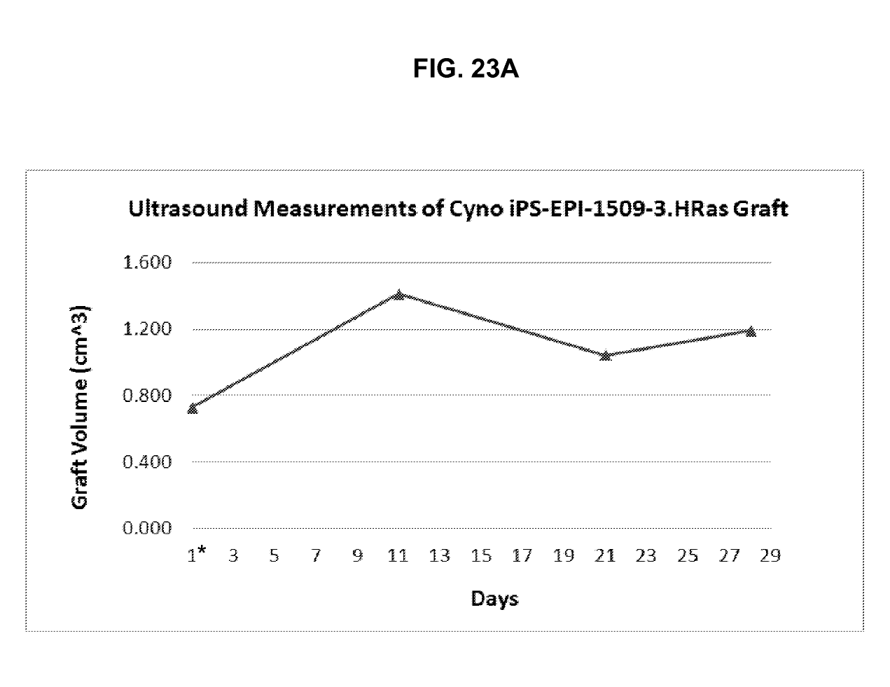

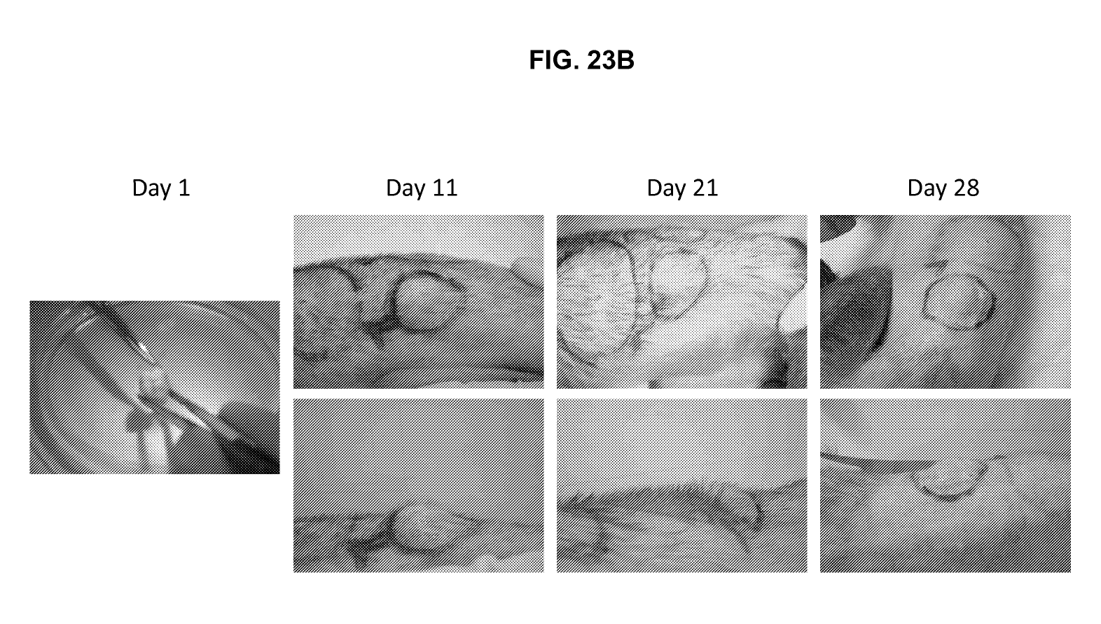

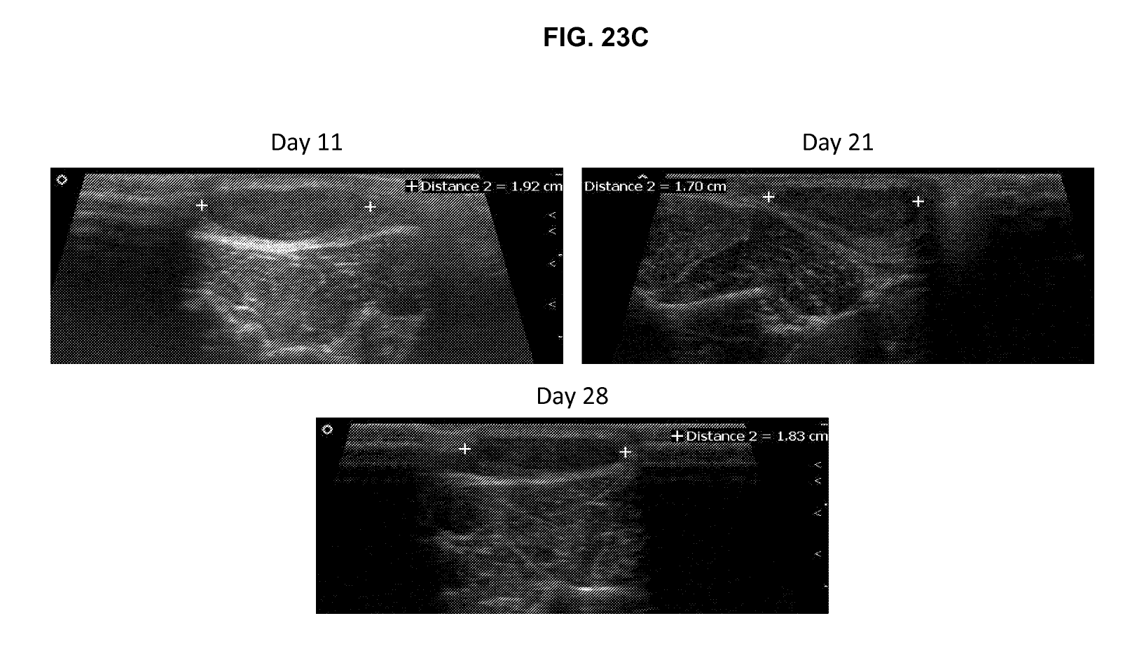

Based on the immunostaining result in comparison between cells and their grafts in FIG. 21, we examined whether the solid cyno iPS-EPI grafts with the enriched cell population in NSG mice might provide better survival in cyno in vivo. The cyno iPS-EPI grafts grown from NSG mice were implanted into the autologous cyno monkey (FIG. 23B) and were measured by ultrasound (FIGS. 23A and 23C). The cyno iPS-EPI-1509-3.HRas graft maintained the similar size from day 1 (pre-implantation) through day 28 after implantation into the autologous cyno 1509 (FIGS. 23A and 23C). This result implies that cyno solid grafts containing the enriched populations can persist better in cyno in vivo.

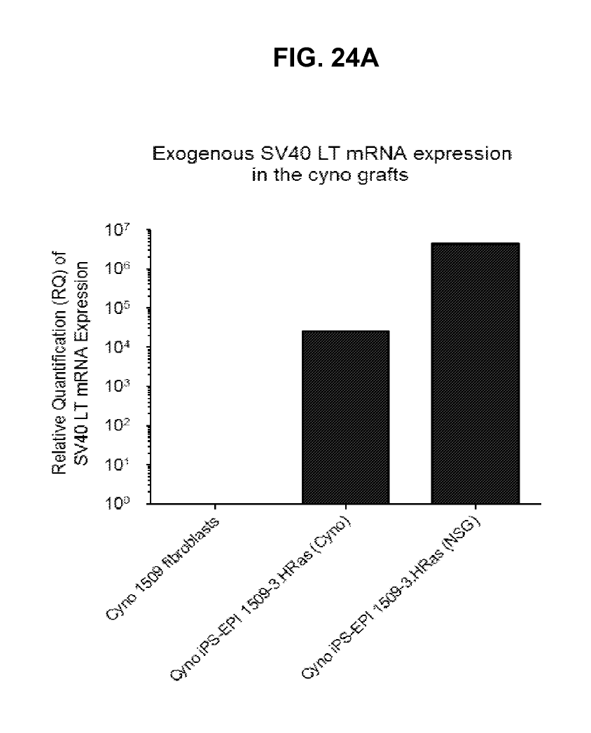

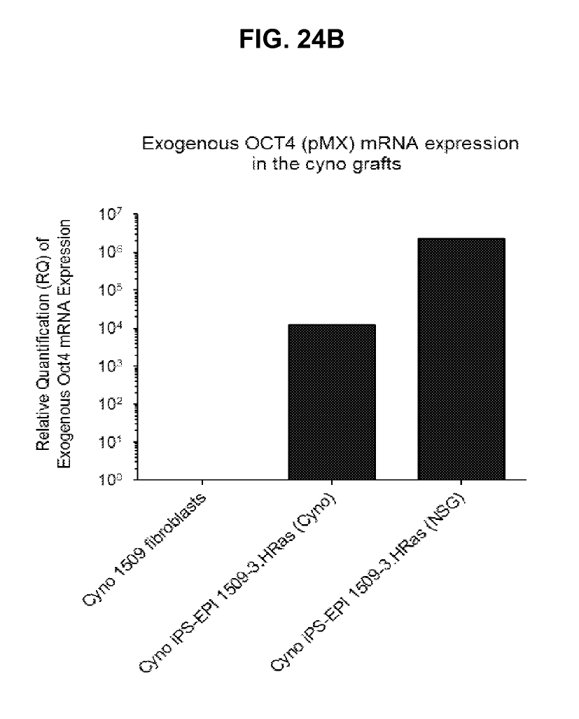

To confirm the presence of cyno iPS-EPI-1509-3.HRas cells in the cyno grafts implanted into the cyno 1509, we performed qPCR using RNA isolated from the cyno graft that was removed from cyno monkey 1509. SV40 LT and an exogenous reprogramming factor, Oct4 (pMX-based), were used to identify the iPS-EPI lines as those genes are not expressed in other endogenous cyno cells in cyno monkeys. The SV40 LT and exogenous Oct4 mRNA expressions were analyzed by qPCR acquiring the relative quantification (RQ) relative to cyno fibroblast obtained from 1509 cyno (FIG. 24A and FIG. 24B, middle bars). The RNA isolated from the cyno iPS-EPI-1509-3.HRas graft that was grown in NSG mice was used as a positive control (FIG. 24A-B, rightmost bars). The large amount of non-iPS-EPI cyno tissues (skin and connective tissues, etc) was included in the harvested cyno tissues, whereas the positive control cyno graft, removed from the NSG mouse site, contained little amount of non-iPS-EPI mouse tissues. Although this large amount of non-iPS-EPI cyno tissues must have diluted the iPS-EPI specific gene expression in the total mRNA, the significantly high expressions of SV40 LT and Oct4 mRNA were detected in cyno iPS-EPI-1509-3.HRas grafts removed from cyno 1509 (FIGS. 24A and 24B), implying the presence of cyno iPS-EPI-1509-3.HRas cells in the cyno graft removed from the cyno monkey.

The autologous target cells or grafts that are injected or implanted subcutaneously, intravenously or by other methods in other suitable area into the original donor cyno monkeys can be examined for efficacies of immune cell engaging therapeutics such as ADCC-mediating antibodies in the autologous setting. Positive control antibodies such as anti-Her2 huIgG1 or anti-CD20 huIgGI antibodies can be administrated into cyno monkeys bearing the HER2- or CD20-expressing cyno iPS-derived cells (e.g., iPS-EPI, foregut, hindgut-like cells) as target cells. Other therapeutic candidate drugs can be tested in this autologous model. After cell injection, blood samples can be periodically withdrawn from the cyno monkeys implanted with the iPS-derived target cells expressing Gluc, and then blood along with coelenterazine can be used to measure the activities of Gluc secreted from the implanted cells. Furthermore, at the same time, the graft or tumor volume can be measured by calipers or ultrasound. In addition, at the end of each study, the grafts or tissues from the injection (or implantation) site will be removed for IHC staining and qPCR (or PCR) to monitor the target cell-specific genes (e.g. SV40 LT and exogenous genes, cMyc, Klf4, Oct4, Sox2, pMX, retroviral vectors), identify the viable injected cells and understand the degree of target cell clearance. Inducible reporter gene (e.g. Gluc) expression can be used as well as constitutive expression of a reporter in case that the reporter may cause the immunogenicity in the tested cyno monkeys. Various cell lines can be injected into the same cyno monkey sequentially and tested, by removal of the previous graft. In addition, the comprehensive studies for efficacies (e.g., target cell clearance or growth suppression of tumors or grafts) can be performed by comparing different variants of antibodies including wild type, afucosylated, and aglycosylated antibodies (human IgG1) or BiTE® or other immune cell engaging therapeutics.

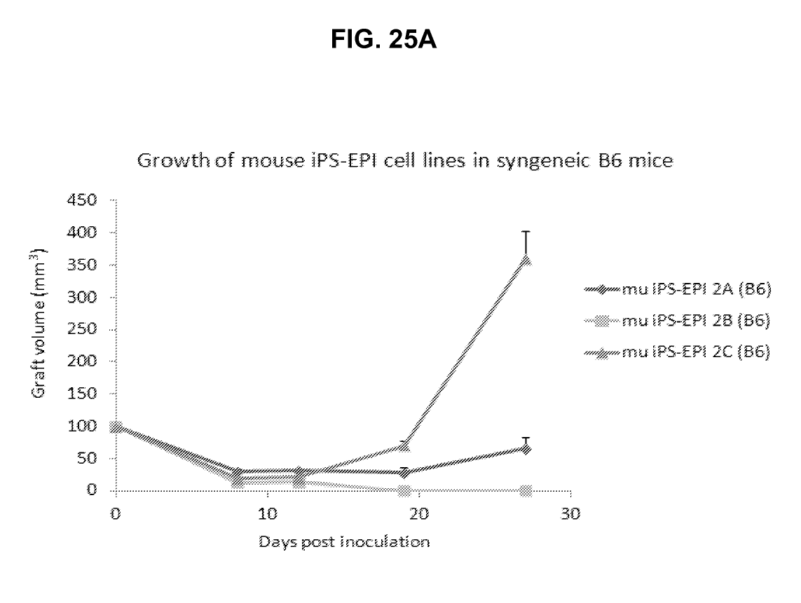

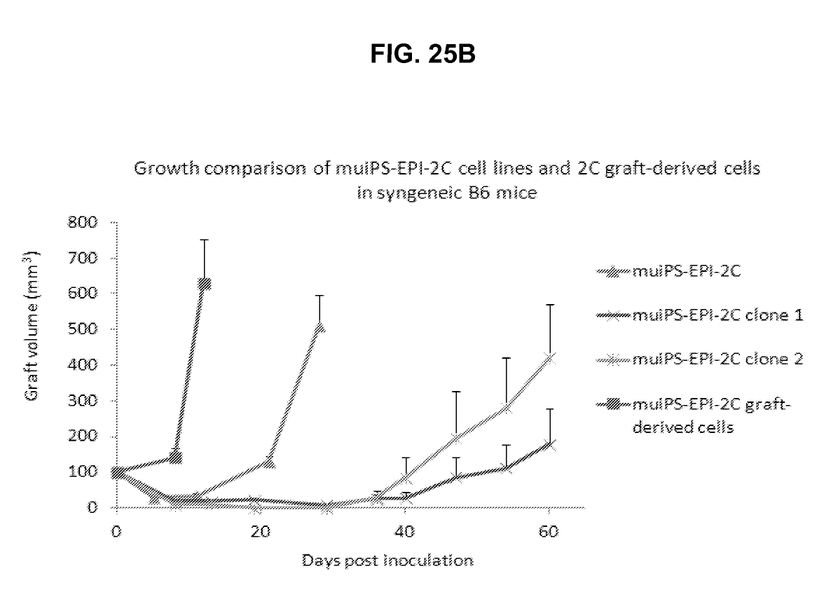

Prior to testing iPS cells-derived autologous target cells in cyno monkeys in vivo, we generated and evaluated mouse iPS cells-derived semi-autologous (syngeneic) models in mice, as a proof of concept. First, we generated mouse iPS cells by reprogramming mouse skin fibroblasts isolated from B6 mouse ears with retroviral transduction of mouse transcription factors, OCT4, SOX2, KLF4, and c-MYC. We next generated epithelial-like cells by differentiating the mouse iPS cells under a serum condition (10% (v/v) FBS) and through multiple passages, which resulted in a heterogeneous, enriched population of epithelial-like cells termed as muiPS-EPI cells. We generated three different muiPS-EPI lines (muiPS-EPI-2A, muiPS-EPI-2B, and muiPS-EPI-2C) with different types of CK expression. Using those lines, we examined the ability of the cell lines to grow and form the grafts in syngeneic B6 mice. The muiPS-EPI-2C formed grafts most effectively in syngeneic B6 mice compared to other cell lines (FIG. 25A). Next, we examined whether the heterogeneity of iPS-EPI cell lines plays an important role in the growth of cells and formation of grafts in vivo. We generated two of single clonal cell lines (muiPS-EPI-2C clone 1 and muiPS-EPI-2C clone 2) that were isolated from muiPS-EPI-2C cell line. The growth rate and ability to form grafts in B6 mice in vivo were significantly reduced in the two clonal populations compared to the original, heterogeneous muiPS-EPI-2C cell line (FIG. 25B), implying that the heterogeneous populations provide better advantages for effective cell growth in B6 mice in vivo. Furthermore, we evaluated the growth ability of cells dissociated from the muiPS-EPI-2C grafts, by injecting those graft-derived cells into the B6 mice (FIG. 25B). The muiPS-EPI-2C graft-derived cells displayed the significantly improved growth rate and the enhanced ability to form the secondary graft compared to the original muiPS-EPI-2C line. This result implies that some selected cell populations may be enriched in the grafts during the cell growth and possibly through the interaction with stromal cells and immune cells in vivo. This strategy of generating the enriched cell populations can provide better survival and growth in the autologous setting in vivo.

The cyno and mouse data disclosed herein demonstrate that the inventive autologous non-human mammalian and primate model systems derived from iPS cells can be used to establish more reliable preclinical models to evaluate the efficacies of potential therapeutics, provide more effective selection of therapeutic candidates for clinical trials, and improve success rates in drug development.

What is claimed:

1. An autologous non-human mammalian model system, comprising:
(i) introducing into a non-human mammal an autologous cell type of interest, wherein the cell type of interest is differentiated from an induced pluripotent stem cell reprogrammed from a primary somatic cell obtained from the non-human mammal, followed by
(ii) administering a therapeutic candidate to the non-human mammal; and then
(iii) determining a physiological effect of the therapeutic candidate in the non-human mammal.

2. The autologous non-human mammalian model system of claim 1, wherein the non-human mammal is a rodent, a rabbit, a dog, a cat, a pig, a sheep, or a non-human primate.

3. The autologous non-human mammalian model system of claim 2, wherein the rodent is a mouse.

4. An autologous non-human primate model system, comprising:
(a) introducing into a non-human primate an autologous cell type of interest, wherein the cell type of interest is differentiated from an induced pluripotent stem (iPS) cell reprogrammed from a primary somatic cell obtained from the non-human primate, followed by
(b) administering a therapeutic candidate to the non-human primate; and then
(c) determining a physiological effect of the therapeutic candidate in the non-human primate.

5. The autologous non-human primate model system of claim 4, wherein the non-human primate is Macaca fascicularis.

6. The autologous non-human primate model system of claim 4, wherein therapeutic candidate is a compound, tool compound, or combination of compounds.

7. The autologous non-human primate model system of claim 4, where the therapeutic candidate is a proteinaceous molecule.

8. The autologous non-human primate model system of claim 7, wherein the proteinaceous molecule is an antigen binding protein.

9. The autologous non-human primate model system of claim 8, wherein the antigen binding protein is an antibody, a bi-specific T-cell engager, or a bi-specific killer cell engager.

10. The autologous non-human primate model system of claim 4, wherein the iPS cell reprogrammed from a primary somatic cell is a fully reprogrammed iPS cell.

11. The autologous non-human primate model system of claim 4, wherein the iPS cell reprogrammed from a primary somatic cell is a partially reprogrammed iPS cell.

12. The autologous non-human primate model system of claim 4, wherein the cell type of interest comprises a target cell.

13. The autologous non-human primate model system of claim 4, wherein the cell type of interest comprises a graft.

14. The autologous non-human primate model system of claim 13, wherein the graft was grown first in another mammal before being transplanted into the autologous non-human primate.

15. The autologous non-human primate model system of claim 12, wherein the target cell comprises an epithelial-like cell, mesenchymal-like, or hematopoietic-like cell.

16. The autologous non-human primate model system of claim 12, wherein the target cell expresses a recombinant gene selected from a tumorigenic gene, an anti-apoptotic gene, an immortalizing gene, and a tumor-related surface antigen.

17. The autologous non-human primate model system of claim 12, wherein the target cell comprises a foregut-like cell, midgut-like cell, or hindgut-like cell.

18. The autologous non-human primate model system of claim 12, wherein the target cell comprises a neuron-like cell or cardiomyocyte.

19. The autologous non-human primate model system of claim 4, wherein the cell type of interest is an effector cell.

20. The autologous non-human primate model system of claim 19, wherein the effector cell is an NK cell.

21. The autologous non-human primate model system of claim 19, wherein the effector cell is a T cell.

22. The autologous non-human primate model system of claim 19, wherein the effector cell is a macrophage, monocyte, or neutrophil.

23. A method of differentiating non-human primate induced pluripotent stem (iPS) cells, in vitro, comprising:
(a) incubating the iPS cells in a cell culture medium comprising a concentration of activin A, while increasing the concentration of serum in the medium from serum-free to about 0.2% (v/v) in the first day and to a final concentration of about 2% (v/v) from the second day onward, effective to induce differentiation of definitive endoderm (DE) cells; and then
(b) culturing the cells in a cell culture medium comprising the concentration of activin A and the final concentration of serum as set forth in (a), for a period of at least twelve days, wherein a population of cells enriched to greater than 90% for SOX2⁺ or PDX1⁺ foregut-like cells results.

24. The method of claim 23, wherein the non-human primate is Macaca fascicularis.

25. A method of differentiating non-human primate induced pluripotent stem (iPS) cells, in vitro, comprising:
(a) incubating the iPS cells for about three days in a cell culture medium comprising a concentration of activin A, while increasing the concentration of serum in the medium from serum-free to about 0.2% (v/v) in the first day and to a final concentration of about 2% (v/v) from the second day onward, effective to induce differentiation of definitive endoderm (DE) cells; and then
(b) culturing the cells in a cell culture medium comprising a concentration of Wnt3a, a concentration of FGF4, and the final concentration of serum as set forth in (a), without added activin A, for a period of at least nine days, wherein a population of cells enriched to greater than 90% for CDX2⁺ hindgut-like cells results.

26. The method of claim 25, wherein the non-human primate is Macaca fascicularis.

27. A non-human primate, comprising an autologous cell type of interest differentiated in vitro from an induced pluripotent stem cell reprogrammed from a primary somatic cell.

28. The non-human primate of claim 27, wherein the non-human primate is Macaca fascicularis.

29. The non-human primate of claim 27, wherein the autologous cell type of interest comprises a target cell.

30. The non-human primate of claim 27, wherein the autologous cell type of interest comprises a graft.

31. The non-human primate of claim 30, wherein the graft was grown first in another mammal before being transplanted into the non-human primate.

32. The non-human primate of claim 29, wherein the target cell comprises an epithelial-like cell or hematopoietic-like cell.

33. The non-human primate of claim 29, wherein the target cell expresses a recombinant gene selected from a tumorigenic gene, an anti-apoptotic gene, an immortalizing gene, and a tumor-related surface antigen.

34. The non-human primate of claim 29, wherein the target cell comprises a foregut-like cell, midgut-like cell, or hindgut-like cell.

35. The non-human primate of claim 29, wherein the target cell comprises a neuron-like cell or cardiomyocyte.

36. The non-human primate of claim 27, wherein the cell type of interest is an effector cell.

37. The non-human primate of claim 36, wherein the effector cell is an NK cell.

38. The non-human primate of claim 36, wherein the effector cell is a macrophage, monocyte, or neutrophil.

39. A non-human primate, comprising an autologous SOX2⁺ or PDX1⁺ foregut-like cell differentiated in vitro by the method of claim 23 from an induced pluripotent stem cell reprogrammed from a primary somatic cell.

40. A non-human primate, comprising an autologous CDX2⁺ hindgut-like cell differentiated in vitro by the method of claim 25 from an induced pluripotent stem cell reprogrammed from a primary somatic cell.

41. A method of monitoring exogenously introduced cells within a non-human mammal, comprising:
(i) introducing into a non-human mammal a recombinant cell that expresses a reporter gene; and
(ii) detecting the reporter gene activity in a tissue sample obtained from the non-human mammal, wherein the level of reporter gene activity is correlated to the number of recombinant cells present in the non-human mammal.

42. The method of claim 41, wherein the non-human mammal is a rodent, a rabbit, a dog, a cat, a pig, a sheep, or a non-human primate.

43. The method of claim 42, wherein the non-human primate is Macaca fascicularis.

44. The method of claim 42, wherein the rodent is a mouse.

45. The method of claim 41, wherein the reporter gene is Gaussia princeps luciferase (Gluc).

46. The method of claim 41, wherein the tissue sample is a blood sample.

47. The method of claim 41, wherein the recombinant cell is comprised in a graft.

48. The method of claim 41, wherein detecting reporter gene activity in the tissue sample comprises measuring mRNA by real time PCR (qPCR) or PCR.

49. The method of claim 41, wherein the recombinant cell is an autologous cell that is a target cell or effector cell type of interest differentiated from an induced pluripotent stem cell reprogrammed from a primary somatic cell.

50. The method of claim 41, wherein the recombinant cell is an autologous cell that is a target cell or effector cell type of interest differentiated from an induced pluripotent stem cell reprogrammed from a primary somatic cell.

51. A method of differentiating non-human primate induced pluripotent stem (iPS) cells, in vitro, comprising co-culturing the iPS cells with stromal cells for at least about thirty days, wherein a population of cells enriched to greater than 10% for CD34⁺ hematopoietic progenitor-like cells results.

52. The method of claim 51, wherein the non-human primate is Macaca fascicularis.

53. A method of differentiating non-human primate induced pluripotent stem (iPS) cells, in vitro, comprising culturing the iPS cells in a cell culture medium comprising a serum concentration of about 10%(v/v), wherein a population of epithelial-like cells results.

54. The method of claim 53, wherein the non-human primate is Macaca fascicularis.

55. A method of monitoring exogenously introduced cells within a non-human mammal, comprising:
(a) introducing into a non-human mammal a recombinant cell that comprises an exogenous gene of interest; and
(b) detecting genomic DNA that is specific to the exogenous gene of interest in a tissue sample obtained from the non-human mammal, wherein the level of genomic DNA that is specific to the exogenous gene of interest is correlated to the number of recombinant cells present in the non-human mammal.

56. The method of claim 55, wherein the non-human mammal is a rodent, a rabbit, a dog, a cat, a pig, a sheep, or a non-human primate.

57. The method of claim 56, wherein the non-human primate is Macaca fascicularis.

58. The method of claim 56, wherein the rodent is a mouse.

59. The method of claim 55, wherein the tissue sample is a blood sample.

60. The method of claim 55, wherein the recombinant cell is comprised in a graft.
